(19) United States
(12) Patent Application Publication
(10) Pub. No.: US 2016/0024559 A1
(43) Pub. Date: Jan. 28, 2016

(54) SPECIMEN COLLECTION WITH REAGENT LINED COLLECTORS

(71) Applicant: The Bode Technology Group, Inc., Lorton, VA (US)

(72) Inventors: Jangbir Sangha, Overland Park, KS (US); Dan Watsula, Centreville, VA (US); Koya D. Reams, Centreville, VA (US); Lana Ramos, Colchester, CT (US)

(21) Appl. No.: 14/807,406

(22) Filed: Jul. 23, 2015

Related U.S. Application Data
(60) Provisional application No. 62/028,459, filed on Jul. 24, 2014.
(60) Provisional application No. 62/028,594, filed on Jul. 24, 2014.
(60) Provisional application No. 62/057,137, filed on Sep. 29, 2014.
(60) Provisional application No. 62/085,782, filed on Dec. 1, 2014.

(51) Int. Cl. C12Q 1/68; G01N 1/40; B01L 3/00

(52) U.S. Cl. CPC C12Q 1/6806; B01L 3/508; G01N 1/405; B01L 2200/16; B01L 2300/16

(57) ABSTRACT

The present disclosure relates to specimen collection devices comprising stabilizing compositions for the collection, preservation, transport, and analysis of evidence samples for forensic analysis.

### CROSS REFERENCE TO RELATED APPLICATIONS

This application claims the priority of U.S. Provisional Patent Application No. 62/028,459, filed Jul. 24, 2014; U.S. Provisional Patent Application No. 62/028,594, filed Jul. 24, 2014; U.S. Provisional Patent Application No. 62/057,137, filed Sep. 29, 2014; and U.S. Provisional Patent Application No. 62/085,782, filed Dec. 1, 2014, each of which is hereby incorporated by reference in its entirety.

### FIELD OF THE INVENTION

The present disclosure relates to devices for the collection of evidence and methods of using such devices. More particularly, the disclosure relates to devices and methods for collection, preservation, transport, and analysis of evidence samples for forensic analysis.

### BACKGROUND OF THE INVENTION

Crime scene evidence is collected to establish facts related to a crime or a suspected crime and for identification or elimination of suspects. Such crime scene evidence may be presented at a trial for the determination of guilt or innocence of accused individuals. Often, the evidence includes objects, documents, fingerprints, photographs of the scene, and the like. Additionally, the evidence may include unknown substances or substances with a suspected identity, where the identity needs to be determined or confirmed. Such substances may be very small in quantity, may be dispersed over a comparatively large area, and may include materials such as body fluids, hairs, flakes of skin, fibers, drugs, various chemicals, gunpowder residue, flammable materials, tobacco ashes, cosmetics, and the like. Such materials may be collected at a scene and subjected to chemical or biomolecule analysis for identification or for association with a particular individual.

For collecting substance samples, investigators typically use fibrous swabs, such as swabs made of fibers of cotton, cellulose, rayon, polyester, and other types of fibers. Such swabs not only absorb liquids and solids entrained in liquids, but also trap dry substances such as particulate materials. The swabs are kept in closed bags or containers prior to use to maintain sterility and are replaced in such containers after use to avoid contamination of the sample gathered. After replacement of a swab in a container, the container is usually marked with a time, the date, and identity of the investigator, as well as other information to establish a chain of custody of the sample.

Conventional swabs are formed of a stick, such as a shaft of wood, tubular plastic, or tubular or rolled paper with a pad of cotton or other fiber, sponge material, or other absorbent material attached to the end of the shaft, either mechanically or by an inert adhesive. A problem with conventional swabs is that there is a danger of contamination of the sample if it is necessary to put the swab down, for example, to open a bag or container in which the swab will be placed. Also, if it is necessary to set the swab down to dry, in a propped up condition or extending over the edge of a table, there is a risk of contamination of the sample.

Another issue relating to the collection of biological specimens is that biological specimens degrade, hindering or affecting downstream analysis. Methods of stabilizing biological specimens used in the forensics community have streamlined the collection and extraction of biomolecules from a variety of samples. These methods include transferring a collected specimen from the collection swab to a stabilizing matrix that harbors storage medium, such as chaotropic salts, that slow down the degradation process. However, such stabilizing methods known in the art are associated with problems. In particular, the collected specimen may not transfer to the stabilizing matrix in a consistent or reproducible manner. Further, if the swab used to collect the sample is separate and distinct from the stabilizing matrix receiving the sample, then forensic traceability issues arise.

Accordingly, a need still exists for a specimen collection device useful for the collection, preservation, transport, and analysis of collected evidence while minimizing the risk of contamination and satisfying chain of custody requirements.

### SUMMARY OF THE INVENTION

In one aspect, a specimen collection device is provided. The specimen collection device comprises a specimen collection absorbent and a reagent lined holder. The reagent of the reagent lined holder is a stabilizing composition deposited in the reagent lined holder and the stabilizing composition is able to transfer to the specimen collection absorbent. The specimen collection device further comprises a means for aligning the specimen collection absorbent with the reagent lined holder, a means for contacting the stabilizing composition with the specimen collection absorbent to deliver the composition to the specimen collection absorbent, and a means for sampling the specimen collection absorbent for further analysis. The device may further comprise a means for maintaining the chain of custody of a collected specimen. The stabilizing reagent may comprise at least one chelating agent, at least one surface acting agent, at least one antimicrobial agent, or combinations thereof.

In some embodiments, the stabilizing composition may comprise at least one chelating agent and at least one surface acting agent. The at least one chelating agent and at least one surface acting agent may be EDTA and Tween®-20. In one embodiment, the stabilizing composition comprises 50 mM EDTA and 0.01% Tween®-20.

In other embodiments, the stabilizing composition may comprise at least one chelator, at least one surface acting agent, and at least one antimicrobial agent. The at least one chelator, at least one surface acting agent, and at least one antimicrobial agent may be EDTA, an azide stabilizer, and Tween®-20. In one embodiment, the stabilizing composition comprises 50 mM EDTA, 0.1% Sodium Azide, and 0.01% Tween®-20.

The stabilizing composition may comprise two chelators. The two chelators may be EDTA and EGTA. The stabilizing composition may comprise two detergents. The two detergents may be Tween and SDS. The preservative composition may comprise one azide stabilizer. The azide stabilizer may be sodium azide. The preservative composition may further comprise a salt and a buffering agent.

In some embodiments, the preservative composition may comprise EDTA, EGTA, Tween, SDS, sodium azide, KCl, and Tris-HCl. In one embodiment, the preservative composition may comprise about 5 to about 15 mM EDTA, about 1 to about 5 mM EGTA, about 0.001% to about 0.1% Tween, about 1 to about 10% SDS, about 0.01 to about 0.1 sodium azide, about 20 to about 30 mM KCl, and about 40 to about 60 mM Tris-HCl.

The stabilizing composition may be in the form of a stabilizing dissolvable film. The stabilizing dissolvable film may comprise at least one film-forming polymer in addition to the stabilizing composition. In some embodiments, the at least one film-forming polymer may be polyvinylpyrrolidone. The polyvinylpyrrolidone may be polyvinylpyrrolidone 40K. The polyvinylpyrrolidone may be present in an amount of about 1% to about 10% by weight in a film-forming solution used to prepare the dissolvable film.

In other embodiments, the at least one water-soluble film-forming polymer is carboxy methyl cellulose. The carboxy methyl cellulose may be present in an amount of about 0.1% to about 1% by weight in a film-forming solution used to prepare the dissolvable film.

The thickness of the film may be about 5μ to about 5 mm. The preservative composition may be present in the film formulation in an amount sufficient to deliver an amount of stabilizing composition sufficient to stabilize a collected specimen. The film formulation may dissolve in about 5 minutes upon contacting a wet sample.

The dissolvable film may also comprise a liner. The liner may be selected from the group consisting of S&S® 903™ paper, S&S® IsoCode® paper, and S&S® 900™, Whatman FTA paper, RAETON™ 16 paper, RAETON™ 26 paper, RAETON™ 96 paper, RAETON™ 7 paper, RG paper, LL72 paper, and B-85 paper.

In another aspect, a specimen collection device is provided. The specimen collection device comprises a specimen collection absorbent and a reagent lined holder. The reagent is a stabilizing composition deposited in the reagent lined holder and the stabilizing composition is able to transfer to the specimen collection absorbent. The specimen collection device further comprises a means for aligning the specimen collection absorbent with the reagent lined holder, a means for contacting the stabilizing composition with the specimen collection absorbent to deliver the composition to the specimen collection absorbent, and a means for sampling the specimen collection absorbent for further analysis. The stabilizing composition comprises 50 mM EDTA and 0.01% Tween®-20. The stabilizing composition may be in the form of a stabilizing dissolvable film.

In yet another aspect, a specimen collection device is provided. The specimen collection device comprises a specimen collection absorbent and a reagent lined holder. The reagent is a stabilizing composition deposited in the reagent lined holder and the stabilizing composition is able to transfer to the specimen collection absorbent. The specimen collection device further comprises a means for aligning the specimen collection absorbent with the reagent lined holder, a means for contacting the stabilizing composition with the specimen collection absorbent to deliver the composition to the specimen collection absorbent, and a means for sampling the specimen collection absorbent for further analysis. The stabilizing composition comprises 50 mM EDTA, 0.1% Sodium Azide, and 0.01% Tween®-20. The stabilizing composition may be in the form of a stabilizing dissolvable film.

In another aspect, a specimen collection device is provided. The device comprises a specimen collection absorbent, and a reagent lined holder. The reagent is a stabilizing composition deposited in the reagent lined holder and is able to transfer to the specimen collection absorbent. The device further comprises a means for aligning the specimen collection absorbent with the reagent lined holder, a means for contacting the stabilizing composition with the specimen collection absorbent to deliver the composition to the specimen collection absorbent, and a means for sampling the specimen collection absorbent for further analysis. The stabilizing composition comprises about 5 to about 15 mM EDTA, about 1 to about 5 mM EGTA, about 0.001% to about 0.1% Tween, about 1 to about 10% SDS, about 0.01 to about 0.1 sodium azide, about 20 to about 30 mM KCl, and about 40 to about 60 mM Tris-HCl. The stabilizing composition may be in the form of a stabilizing dissolvable film.

In an additional aspect, a method of collecting a specimen for analysis is provided. The method comprises the steps of: providing a specimen collector having a specimen collection absorbent and a reagent lined cassette; contacting the specimen collection absorbent to a specimen for collection; closing the specimen collector, wherein the sample collection absorbent is moved into a position that aligns the specimen collection absorbent with the reagent lined cassette; contacting the specimen collection absorbent to the reagent lined cassette to deliver the reagent to the specimen collection absorbent; storing the specimen collector; and sampling the specimen collection absorbent for analysis.

### BRIEF DESCRIPTION OF THE DRAWINGS

The following drawings form part of the present disclosure and are included to further demonstrate certain aspects of the present disclosure. The disclosure may be better understood by reference to one or more of these drawings in combination with the detailed description of specific aspects presented herein. The drawings are not to scale.

FIG. 1 depicts an embodiment of a specimen collection device having a storage holder and a specimen collector shown in the assembled configuration with the specimen collector mounted within the storage holder.

FIG. 2 depicts the embodiment of the specimen collection device of FIG. 1 showing the specimen collector separated from the storage holder and showing the collection area on the specimen collector, and the reagent area on the storage holder.

FIG. 3 depicts an embodiment of a specimen collection device having a storage holder and specimen collector mounted therein wherein the specimen collector is equipped with a handle.

FIG. 4 depicts the embodiment of the specimen collection device shown in FIG. 3 shown in exploded view and showing the reagent area on the storage holder and the handle slots for receiving handle pins of the handle.

FIG. 5 depicts a side view of the handle of the specimen collection device shown in FIG. 3, showing the handle pins of the handle.

FIG. 6 depicts a front view of the handle of the specimen collection device shown in FIG. 3, showing the handle pins of the handle.

FIG. 7 depicts an embodiment of a specimen collection device having a storage holder and a specimen collector shown in the assembled configuration with the specimen collector mounted within the storage holder.

FIG. 8 depicts the specimen collector of the specimen collection device of FIG. 7 shown separated from the storage holder and showing the removable cover placed over the surface of the specimen collector.

FIG. 9 depicts the storage holder of the specimen collection device of FIG. 7 shown separated from the specimen collector and showing the removable liner provided to cover a reagent area on the storage holder.

FIG. 10 depicts a cassette which is used for long-term storage of any of the collection and storage devices described above, showing a specimen collection device inserted into the cassette, and a specimen window shown covered by a cassette liner.

FIG. 11 depicts the cassette of FIG. 10 with the cassette liner removed exposing the specimen window and the collection area of a specimen collection device stored in the storage holder.

FIG. 12 depicts an embodiment of a specimen collection device having a collector frame being rotatable between an open exposed position shown and a closed protected position in a protective storage holder. The specimen collection device is shown in an open exposed position with the collector frame extending from the storage holder.

FIG. 13 depicts the embodiment of the specimen collection device of FIG. 12 showing the rotation of the collector frame into the storage holder.

FIG. 14 depicts the embodiment of the specimen collection device shown in FIG. 12 in a closed protected position with the collector frame rotated into the storage holder.

FIG. 15 depicts the embodiment of the specimen collection device shown in FIG. 12 in a closed protected position and showing the collector paper within the storage holder and the flap(s) removed.

FIG. 16 depicts an embodiment of a specimen collection device having an elongated support frame having the collector paper mounted on one end and the protective storage holder is slideable along the length of the support frame to either expose the absorbent collector paper or to enclose it within the storage holder. The specimen collection device is shown with the protective storage holder slid upward and enclosing the collector paper within the storage holder.

FIG. 17 depicts the embodiment of the specimen collection device of FIG. 16 shown with the protective storage holder slid downward to expose the absorbent collector paper.

FIG. 18 depicts the embodiment of the specimen collection device of FIG. 16 showing removable flaps on front and back, and perforations on the support frame to detach the handle portion of the support frame if desired after specimen collection.

FIG. 19 depicts an embodiment of the specimen collection device of FIG. 16 having perforations on the support frame and striations on the bottom edge of the storage holder to provide further assistance in removing the handle portion of the support frame if desired after specimen collection. The specimen collection device is shown with the protective storage holder slid upward and enclosing the collector paper within the storage holder and the flap(s) removed.

FIG. 20 depicts an embodiment of a specimen collection device comprising a handle with collector paper and having a slider mounted thereon and capable of moving forwardly and backwardly on the handle to either expose or to cover the collector paper mounted on a support. The specimen collection device is shown with the handle and slider moved backwardly.

FIG. 21 depicts the embodiment of the specimen collection device of FIG. 20 in position for collection of a specimen thereon with the handle advanced within the support to position the collector paper and a removable barrier sheet over the reagent pad.

FIG. 22 depicts the embodiment of the specimen collection device of FIG. 20 in a position after the specimen has been collected onto the collector paper with the slider advanced to cover the collector paper before removing the removable barrier sheet.

FIG. 23 depicts the embodiment of the specimen collection device of FIG. 20 in a position after the specimen has been collected onto the collector paper with the slider advanced to cover the collector paper with the removable barrier sheet removed.

FIG. 24 depicts the embodiment of the specimen collection device of FIG. 20 in a position after the specimen has been collected onto the collector paper with the slider secured in contact with the support.

FIG. 25 depicts an embodiment of a specimen collection device comprising a handle base with collector paper and having a slider cover mounted thereon and capable of moving forwardly and backwardly on the handle to either expose or to cover the collector paper mounted on a support and a protective cap placed over the support to cover the reagent pad. The specimen collection device is shown with the handle and slider moved backwardly.

FIG. 26 depicts the embodiment of the specimen collection device of FIG. 25 in position for collection of a specimen thereon with the handle advanced within the support to position the collector paper over a protective cap placed over the support to cover the reagent pad.

FIG. 27 depicts the embodiment of the specimen collection device of FIG. 25 in position after collection of a specimen thereon with the slider moved upwardly in the direction of the arrow to bring the slider cover into contact with the lower end of the protective cap.

FIG. 28 depicts the embodiment of the specimen collection device of FIG. 25 in position after collection of a specimen thereon and protective cap two removed from the support and placing the collector paper in contact with the reagent pad and secured by protective cap one. The specimen collection device is shown with the handle and slider moved backwardly. Ventilation holes on protective cap one allow passage of air between the outside environment and the collector paper to assist in drawing of the specimen on the collector paper.

FIG. 29 depicts an exploded view of an embodiment of a specimen collection device comprising a handle base with collector paper and having a slider cover mounted thereon and capable of moving forwardly and backwardly on the handle to either expose or to cover the collector paper mounted on a support, the handle and slider mounted into a support having a reagent pad mounted thereon and protected by a protective cap.

FIG. 30 depicts the embodiment of the specimen collection device of FIG. 29 in position for collection of a specimen thereon with the handle advanced within the support to position the collector paper over the protective cap placed over the support to cover the reagent pad.

FIG. 31 depicts the embodiment of the specimen collection device of FIG. 29 in position after collection of a specimen thereon and the protective cap removed from the support, placing the collector paper in contact with the reagent pad and showing a plug on the slider and a receptacle on the support, the receptacle being of complimentary design to receive the plug in the frictional fit to secure the slider against the support to prevent further movement of the slider and to lock the slider into a protective covering position over the collector paper.

FIG. 32 depicts the embodiment of the specimen collection device of FIG. 29 in position after collection of a specimen thereon having the protective cap removed from the support, and the plug in the frictional fit in the receptacle to secure the slider against the support, preventing further movement of the slider and locking the slider into a protective covering position over the collector paper.

FIG. 33 depicts a top perspective view of the interconnection between the projection on the slider and the notch or void of the support in the embodiment of the specimen collection device of FIG. 29.

FIG. 34 depicts a side view of the projection positioned over the reagent pad prior to its insertion into the notch or void of support in the embodiment of the specimen collection device of FIG. 29.

FIG. 35 depicts an exploded front view of an embodiment of a specimen collection device comprising a handle with collector paper and a slider cover mounted thereon and capable of moving forwardly and backwardly on the handle to either expose or to cover the collector paper mounted on the handle, the handle and slider mounted into a support. In this embodiment, the reagent pad is mounted on the slider cover.

FIG. 36 depicts an exploded back view of the embodiment of a specimen collection device shown in FIG. 35, showing the reagent pad mounted on the slider cover and a shrink wrap barrier covering the reagent pad, the covering having a tab extending from the cap barrier to permit removal of the shrink wrap barrier from the reagent pad by a user when it is desired to apply the reagents to the absorbent collector paper.

FIG. 37 depicts the embodiment of the specimen collection device of FIG. 35 in position for collection of a specimen.

FIG. 38 depicts the embodiment of the specimen collection device of FIG. 35 in position after collection of a specimen thereon having the protective cap removed from the slider to expose the reagent pad, and a plug in the frictional fit in the receptacle to secure the slider against the support, preventing further movement of the slider and locking the slider into a protective covering position over the collector paper.

FIG. 39 depicts an exploded view of an embodiment of a specimen collection device comprising a handle having collector paper extending therefrom, a slider equipped on its end with a snap-in void mateable with a projection extending from a cap slidably connected to the handle and comprising a reagent pad.

FIG. 40 depicts the specimen collection device of FIG. 39 in position for collection of a specimen.

FIG. 41 depicts the specimen collection device of FIG. 39 with the protective cap and the reagent pad moved into a position over the absorbent paper after a specimen has been collected. The device is shown with a projection on the protective cap securing the protective cap against the slider to provide secure coverage of the collector paper to guard against contamination of the collector paper during transportation or storage.

FIG. 42 depicts the specimen collection device of FIG. 39 with the protective cap and the reagent pad moved over the absorbent paper after a specimen has been collected but before securing the protective cap against the slider. The specimen collection device also comprises an RFID chip inserted into the device.

FIG. 43 depicts an embodiment of a specimen collection device comprising a slider having a collector paper mounted thereon and a protective cap comprising a reagent pad. The protective cap and reagent pad can be moved into a position over the absorbent paper. The specimen collection device is shown in position for collection of a specimen.

FIG. 44 depicts the embodiment of the specimen collection device shown in FIG. 43 with the protective cap and the reagent pad moved into a position over the absorbent paper after a specimen has been collected.

FIG. 45 depicts an embodiment of the specimen collection device shown in FIG. 43, wherein the specimen collection device further comprises a front ridge to compress the slider against the reagent pad of the cap.

FIG. 46 graphically illustrates the amount of saliva absorbed per absorbent condition.

FIG. 47 graphically illustrates the percentage of DNA recovered per absorbent condition.

FIG. 48 graphically illustrates the percentage of DNA recovered per absorbent condition in comparison to a frozen DNA control sample.

FIG. 49 graphically illustrates the stability of DNA collected using a device described herein with a preservative following storage for 8 weeks at room temperature.

FIG. 50 graphically illustrates DNA stability and concentration of samples following storage for 5 days at room temperature under various reagent conditions.

FIG. 51 graphically illustrates DNA stability and concentration of samples following storage for 20 days at room temperature under various reagent conditions.

FIG. 52 graphically illustrates DNA stability and concentration of samples following storage for 2, 4, and 6 weeks at room temperature.

FIG. 53 graphically illustrates DNA stability and concentration of samples under various reagent conditions.

FIG. 54 graphically illustrates the CSF/D8 pHT ratios for saliva samples under various reagent conditions.

FIG. 55 graphically illustrates the percentage of DNA recovered per absorbent condition in comparison to a frozen DNA control sample.

FIG. 56 shows a picture of the dissolvable film solution placed in a dehydrator to produce the film.

FIG. 57 is a photo of a Specimen Information Card (SIC) with an arrow indicating the window to be covered by the dissolvable film.

FIG. 58 shows a photo of an adhesive label used to attach the dissolvable film to the SIC.

FIG. 59 shows a photo of the dissolvable film attached to the SIC.

FIG. 60 shows a photo of the SIC inverted using a specimen collector. The inverted SIC is placed film side down onto the collected sample using the slider of the specimen collector to help secure the card in place.

FIG. 61 is a photo of the front side of the collection absorbent after the dissolvable film has dissolved onto the collection absorbent.

FIG. 62 is a photo of the back side of the collection absorbent after the dissolvable film has dissolved onto the collection absorbent.

FIG. 63 shows a photo of an archival cassette after the dissolvable film was dissolved and the collector stored.

FIG. 64 shows a photo of an archival specimen collector after the dissolvable film was dissolved and the collector stored.

FIG. 65 shows a photo of an archival cassette after the dissolvable film was dissolved and the collector stored.

FIG. 66 shows a photo of an archival specimen collector after the dissolvable film was dissolved and the collector stored.

FIG. 67 is a photo of the front side of the collection absorbent after the dissolvable film has dissolved onto the collection absorbent.

FIG. 68 is a photo of the back side of the collection absorbent after the dissolvable film has dissolved onto the collection absorbent.

FIG. 69 is a photo of a collection absorbent split down the middle.

FIG. 70 depicts a photograph showing paper, glass fiber, and woven polyester material cut into the shape of the paper in a Buccal DNA Collector™.

FIG. 71 depicts a photograph showing paper, glass fiber, and woven polyester material with 5% PVP 10K solution applied thereon, immediately after application of the solution (A), about 7½ minutes after application of the solution (B), about 26 minutes after application of the solution (C), and about 1 hour and 20 minutes after application of the solution (D).

FIG. 72 depicts paper, glass fiber, and woven polyester material with 5% PVP 40K solution applied thereon, immediately after application of the solution (A), about 7½ minutes after application of the solution (B), about 26 minutes after application of the solution (C), and about 1 hour and 20 minutes after application of the solution (D).

FIG. 73 depicts paper, glass fiber, and woven polyester material with 0.25% CMC solution applied thereon, immediately after application of the solution (A), about 7½ minutes after application of the solution (B), about 26 minutes after application of the solution (C), and about 1 hour and 20 minutes after application of the solution (D).

FIG. 74 depicts paper, glass fiber, and woven polyester material with 0.5% CMC solution applied thereon, immediately after application of the solution (A), about 7½ minutes after application of the solution (B), about 26 minutes after application of the solution (C), and about 1 hour and 20 minutes after application of the solution (D).

FIG. 75 depicts photographs showing the dried PVP 10K PROTECT solutions on woven polyester being placed on top of the sample collector paper (A), and the sample collector after transfer of PROTECT solution (B).

FIG. 76 depicts photographs showing the dried CMC 0.25% PROTECT solutions on woven polyester being placed on top of the sample collector paper (A), and the sample collector after transfer of PROTECT solution (B).

FIG. 77 depicts photographs showing dried PVP 5% PROTECT solutions on woven polyester, a liner, and a sample collector (A), dried PVP 5% PROTECT solutions on woven polyester and a sample on a sample collector (B), application of PROTECT solution with or without a liner (C), and the sample collectors after transfer of PROTECT solution without using a liner (D), or with liner (E).

FIG. 78 depicts photographs showing dried CMC 0.25% PROTECT solutions on woven polyester, a liner, and a sample on a sample collector (A), dried CMC 0.25% PROTECT solutions on woven polyester and a sample on a sample collector (B), and the sample collectors after transfer of PROTECT solution with liner (C), or without using a liner (D).

FIG. 79 depicts photographs showing dried sample collectors with 5% PVP PROTECT 10K solution (A) and 5% PVP PROTECT 40K solution (B) on paper, and the sample collectors after transfer of PVP PROTECT 10K solution (C) or the sample collectors after transfer of PVP PROTECT 40K solution (D).

FIG. 80 depicts photographs showing a container comprising 40K PVP PROTECT solution and paper (A), the paper after submersion into the solution (B), the submerged paper after drying (C), a Buccal DNA sample Collector™ and the dried paper cut into the shape of the paper in a Buccal DNA Collector™ (D), and the sample collectors after transfer of PVP PROTECT 40K solution (E).

FIG. 81 depicts photographs showing Buccal DNA sample Collectors™ and paper submerged in 40K PVP PROTECT solution, dried, and cut into the shape of the paper in a Buccal DNA Collector™ (A and B), and the sample collectors after transfer of PVP PROTECT 40K solution (C and D).

FIG. 82 depicts photographs showing dried sample collectors with 0.5% CMC PROTECT solution (A) and 0.25% CMC PROTECT solution (B) on paper, and the sample collectors after transfer of 0.5% CMC PROTECT solution (C) or transfer of 0.25% CMC PROTECT solution (D).

FIG. 83 depicts photographs showing RG paper cut into the shape of the paper in a Buccal DNA Collector™ (A), RG paper with PVP 40K, PVP 10K, 0.25% CMC, and 0.5% CMC solution applied thereon immediately after application of the solution (B), and the RG paper in (B) about 7 hours after application of the various solutions (C).

FIG. 84 depicts photographs showing LL72 paper cut into the shape of the paper in a Buccal DNA Collector™ (A), LL72 paper with PVP 40K, PVP 10K, 0.25% CMC, and 0.5% CMC solution applied thereon immediately after application of the solution (B), and the LL72 paper in (B) about 7 hours after application of the various solutions.

FIG. 85 depicts photographs showing B-85 paper cut into the shape of the paper in a Buccal DNA Collector™ (A), B-85 paper with PVP 40K, PVP 10K, 0.25% CMC, and 0.5% CMC solution applied thereon immediately after application of the solution (B), and the B-85 paper in (B) about 7 hours after application of the various solutions.

FIG. 86 depicts photographs showing LL72 paper with PVP 40K solution and a Buccal DNA Collector™ (A), and the Buccal DNA Collector™ after contacting with the LL72 paper (B).

FIG. 87 shows 4 photos of collection absorbents after the dissolvable film has dissolved onto the collection absorbents, and after a number of 1.2 mm punches were taken from each collector for sample analysis.

FIG. 88 depicts photographs showing RAETON™ paper cut into the shape of the paper in a Buccal DNA Collector™.

FIG. 89 depicts RAETON™ 16 (A), RAETON™ 26 (B), RAETON™ 96 (C), and original paper (D) paper with 5% PVP 40K solution with 20% IPA applied thereon, immediately after application of the solution.

FIG. 90 depicts RAETON™ 16 (A), RAETON™ 26 (B), RAETON™ 96 (C), and original paper (D) paper with 5% PVP 40K solution with 20% IPA applied thereon, about 7 minutes after application of the solution.

FIG. 91 depicts RAETON™ 16 (A), RAETON™ 26 (B), RAETON™ 96 (C), and original paper (D) paper with 5% PVP 40K solution with 20% IPA applied thereon, about 20 minutes after application of the solution.

FIG. 92 depicts RAETON™ 16 (A), RAETON™ 26 (B), RAETON™ 96 (C), and original paper (D) paper with 5% PVP 40K solution with 20% IPA applied thereon, about 1 hour and 40 minutes after application of the solution.

FIG. 93 depicts RAETON™ 16 (A), RAETON™ 26 (B), RAETON™ 96 (C), and original paper (D) paper with 5% PVP 40K solution with 20% IPA applied thereon, about 6½ hours after application of the solution.

FIG. 94 depicts photographs showing PROTECT film on RAETON™ 16 and 26 paper remaining tacky (arrow) as observed when stored in a plastic bag.

FIG. 95 depicts photographs showing Buccal DNA sample Collectors™ and RAETON™ 16 paper with 40K PVP PROTECT with 20% IPA film cut into the shape of the paper in a Buccal DNA Collector™ (A), and the sample collectors after transfer of PVP PROTECT 40K+IPA (B).

FIG. 96 depicts photographs showing Buccal DNA sample Collectors™ and RAETON™ 26 paper with 40K PVP PROTECT with 20% IPA film cut into the shape of the paper in a Buccal DNA Collector™ (A), and the sample collectors after transfer of PVP PROTECT 40K+IPA (B).

FIG. 97 depicts photographs showing Buccal DNA sample Collectors™ and original paper with 40K PVP PROTECT with 20% IPA film cut into the shape of the paper in a Buccal DNA Collector™ (A), and the sample collectors after transfer of PVP PROTECT 40K+IPA (B).

FIG. 98 depicts photographs showing Buccal DNA sample Collectors™ and RAETON™ 96 with 40K PVP PROTECT with 20% IPA film cut into the shape of the paper in a Buccal DNA Collector™ (A), and the sample collectors after transfer of PVP PROTECT 40K+IPA (B).

FIG. 99 depicts photographs showing RAETON™ 26 paper cut into the shape of the paper in a Buccal DNA Collector™ with 5% PVP 40K solution with or without 20% IPA applied thereon, immediately after application of the solution (A), about 8 minutes after application of the solution (B), about 20 minutes after application of the solution (C), and about 2 hours after application of the solution (D). In each frame, the three papers on the left have 5% PVP 40K solution with 20% IPA applied thereon, and the three papers on the right have 5% PVP 40K solution without IPA applied thereon.

FIG. 100 depicts photographs showing Buccal DNA sample Collectors™ and RAETON™ 26 paper with 40K PVP PROTECT without 20% IPA film cut into the shape of the paper in a Buccal DNA Collector™ (A), the sample collectors after transfer of PVP PROTECT 40K without IPA (B), RAETON™ 26 paper with 40K PVP PROTECT with 20% IPA film cut into the shape of the paper in a Buccal DNA Collector™ (C), and the sample collectors after transfer of PVP PROTECT 40K with IPA (D).

FIG. 101 depicts photographs showing Buccal DNA sample Collectors™ with buccal sample, and RAETON™ 16 paper with 40K PVP PROTECT with 20% IPA film cut into the shape of the paper in a Buccal DNA Collector™ (A), and the sample collectors after transfer of PVP PROTECT 40K+IPA (B).

FIG. 102 depicts photographs showing Buccal DNA sample Collectors™ with buccal sample, and RAETON™ 26 paper with 40K PVP PROTECT with 20% IPA film cut into the shape of the paper in a Buccal DNA Collector™ (A), and the sample collectors after transfer of PVP PROTECT 40K+IPA (B).

FIG. 103 depicts photographs showing Buccal DNA sample Collectors™ with buccal sample, and RAETON™ 26 paper with 40K PVP PROTECT without 20% IPA film cut into the shape of the paper in a Buccal DNA Collector™ (A), and the sample collectors after transfer of PVP PROTECT 40K (B).

FIG. 104 depicts photographs showing Buccal DNA sample Collectors™ with buccal sample, and original paper with 40K PVP PROTECT without 20% IPA film cut into the shape of the paper in a Buccal DNA Collector™ (A), and the sample collectors after transfer of PVP PROTECT 40K (B).

FIG. 105 depicts a photograph showing a pipette tip with the end cut off to generate a wider pipetting end (A), and photographs showing three different batches of RAETON™ 26, RAETON™ 7, and Joanne paper cut into the shape of the paper in a Buccal DNA Collector™ (B).

FIG. 106 depicts photographs showing three different batches of RAETON™ 26 (A, B, C), RAETON™ 7 (B), and Joanne (C) paper liners with 5% PVP 40K solution applied thereon immediately after application of the solution. In each frame, the three paper liners on the left have 100 μl solution pipetted using 3 pipette tips, 33.3 μl each, and the two paper liners on the right have 100 μl solution pipetted into a single drop using one pipette tip which has been cut to give a wider pipetting end.

FIG. 107 depicts photographs showing three different batches of RAETON™ 26 (A, B, C), RAETON™ 7 (D), and Joanne (E) paper with 5% PVP 40K solution applied thereon about 12 minutes after application of the solution. In each frame, the three paper liners on the left have 100 μl solution pipetted using 3 pipette tips, 33.3 μl each, and the two paper liners on the right have 100 μl solution pipetted into a single drop using one pipette tip which has been cut to give a wider pipetting end.

FIG. 108 depicts photographs showing three different batches of RAETON™ 26 (A, B, C), RAETON™ 7 (D), and Joanne (E) paper with 5% PVP 40K solution applied thereon about 1 hour after application of the solution. In each frame, the three paper liners on the left have 100 μl solution pipetted using 3 pipette tips, 33.3 μl each, and the two paper liners on the right have 100 μl solution pipetted into a single drop using one pipette tip which has been cut to give a wider pipetting end.

FIG. 109 depicts photographs showing three different batches of RAETON™ 26 (A, B, C), RAETON™ 7 (D), and Joanne (E) paper with 5% PVP 40K solution applied thereon about 3.5 hours after application of the solution. In each frame, the three paper liners on the left have 100 μl solution pipetted using 3 pipette tips, 33.3 μl each, and the two paper liners on the right have 100 μl solution pipetted into a single drop using one pipette tip which has been cut to give a wider pipetting end.

FIG. 110 depicts photographs showing three different batches of RAETON™ 26 (A, B, C), RAETON™ 7 (D), and Joanne (E) paper with 5% PVP 40K solution applied thereon about 22 hours after application of the solution. In each frame, the three paper liners on the left have 100 μl solution pipetted using 3 pipette tips, 33.3 μl each, and the two paper liners on the right have 100 μl solution pipetted into a single drop using one pipette tip which has been cut to give a wider pipetting end.

FIG. 111 depicts photographs showing Buccal DNA sample Collectors™ moistened with water, and RAETON™ 26 paper BATCH #1 with one 100 μl drop of 40K PVP PROTECT film (A), and the sample collector after transfer of PVP PROTECT 40K (B).

FIG. 112 depicts photographs showing Buccal DNA sample Collectors™ moistened with water, and RAETON™ 26 paper BATCH #1 with three drops of 33.3 μl each of 40K PVP PROTECT film (A), and the sample collectors after transfer of PVP PROTECT 40K (B).

FIG. 113 depicts photographs showing Buccal DNA sample Collectors™ moistened with water, and RAETON™ 7 paper with three drops of 33.3 μl each of 40K PVP PROTECT film (A), and the sample collector after transfer of PVP PROTECT 40K (B).

FIG. 114 depicts photographs showing Buccal DNA sample Collectors™ moistened with water, and Joanne paper with three drops of 33.3 μl each of 40K PVP PROTECT film (A), and the sample collectors after transfer of PVP PROTECT 40K (B).

FIG. 115 depicts photographs showing Buccal DNA sample Collectors™ moistened with water, and RAETON™ 26 batch #2 paper with three drops of 33.3 μl each of 40K PVP PROTECT film (A), and the sample collectors after transfer of PVP PROTECT 40K (B).

FIG. 116 depicts photographs showing Buccal DNA sample Collectors™ moistened with water, and RAETON™ 26 batch #3 with three drops of 33.3 μl each of 40K PVP PROTECT film (A), and the sample collectors after transfer of PVP PROTECT 40K (B).

FIG. 117 depicts photographs showing Buccal DNA sample Collectors™ with buccal sample, and RAETON™ 26 paper BATCH #1 with one 100 μl drop of 40K PVP PROTECT film (A), and the sample collector after transfer of PVP PROTECT 40K (B).

FIG. 118 depicts photographs showing Buccal DNA sample Collectors™ with buccal sample, and RAETON™ 26 paper BATCH #1 with three drops of 33.3 μl each of 40K PVP PROTECT film (A), and the sample collectors after transfer of PVP PROTECT 40K (B).

FIG. 119 depicts photographs showing Buccal DNA sample Collectors™ with buccal sample, and RAETON™ 26 paper BATCH #1 with three drops of 33.3 μl each of 40K PVP PROTECT film (A), and RAETON™ 7 paper with three drops of 33.3 μl each of 40K PVP PROTECT film (B), textbooks stacked on top of collectors with liners applied thereon (C), and the sample collectors after transfer of PVP PROTECT 40K from the RAETON™ 26 paper BATCH #1 (D), and RAETON™ 7 paper (E).

### DETAILED DESCRIPTION

A specimen collection device and specimen-stabilizing compositions useful for the collection, preservation, transport, and analysis of collected biological evidence have been developed. A specimen collection device of the present disclosure comprises a specimen collection absorbent and a reagent lined area, wherein the reagent is a stabilizing composition deposited in the reagent lined holder. The specimen collection device comprises means for aligning the specimen collection absorbent with the reagent lined area to contact the stabilizing composition with the specimen collection absorbent and deliver or transfer the stabilizing composition to the specimen collection absorbent.

Advantageously, a stabilizing formulation of the present specimen collection device is capable of slowing down sample degradation, preserving biomolecule contents of collected samples, providing for efficient penetration into a sample, and allowing for hands-free application of a sufficient amount of a stabilizing composition without interfering with subsequent sample testing steps. The invention also allows for collecting biological samples, while later permitting extraction of portions of the sample collection absorbent for testing while minimizing the risk of contamination and satisfying chain of custody requirements.

### I. Collection Devices

In one aspect, the present disclosure provides various embodiments of an evidence collection device with a reagent lined holder. An evidence collection device of the present disclosure comprises a specimen collection absorbent on a specimen collector and a reagent lined holder. The reagent is a stabilizing composition deposited in the reagent lined holder. The evidence collection device comprises a means for aligning the specimen collection absorbent with the reagent lined holder, a means to apposition and contact the stabilizing composition with the specimen collection absorbent to efficiently deliver the stabilizing composition to the specimen collection absorbent, and a means for sampling the specimen collection absorbent for further analysis. As used herein, the terms “deliver” and “transfer” may be used interchangeably and refer to the transfer or delivery of stabilizing solution from the reagent lined area to the specimen collection absorbent, wherein the transfer of stabilizing reagent to the specimen collection absorbent is of an amount sufficient to stabilize a collected specimen on a specimen collection absorbent. Transfer of stabilizing reagent to the specimen collection absorbent may be enhanced by providing means for ventilation for efficient evaporation of a collected specimen, which evaporation acts to draw the stabilizing reagent from the reagent lined holder and onto the specimen collection absorbent comprising the specimen. For instance, means for ventilation may be provided by gaps or holes in the device to allow for efficient evaporation of a collected specimen.

All embodiments of the evidence collection device may also comprise means for maintaining the chain of custody of a collected specimen. Means for maintaining the chain of custody of a collected specimen are known in the art and may include identification indicia, radio-frequency identification (RFID) tags and the like. An RFID chip could be a passive or active chip and would have a unique identification number associated therewith to provide a unique identifier to each individual specimen collector. A stabilizing composition may be as described in Sections II and III below.

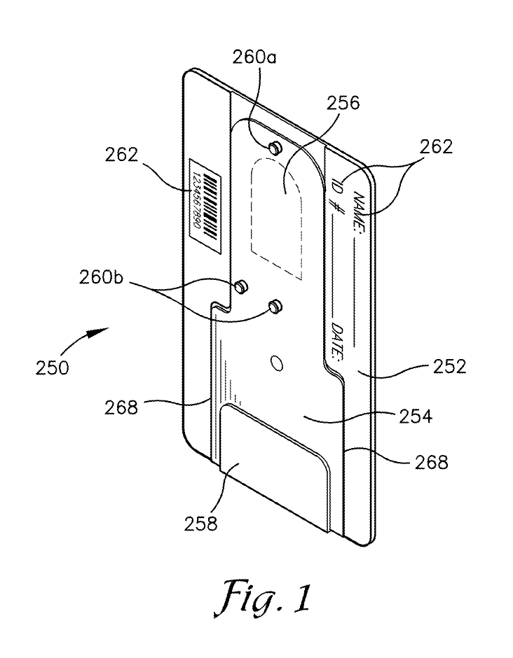

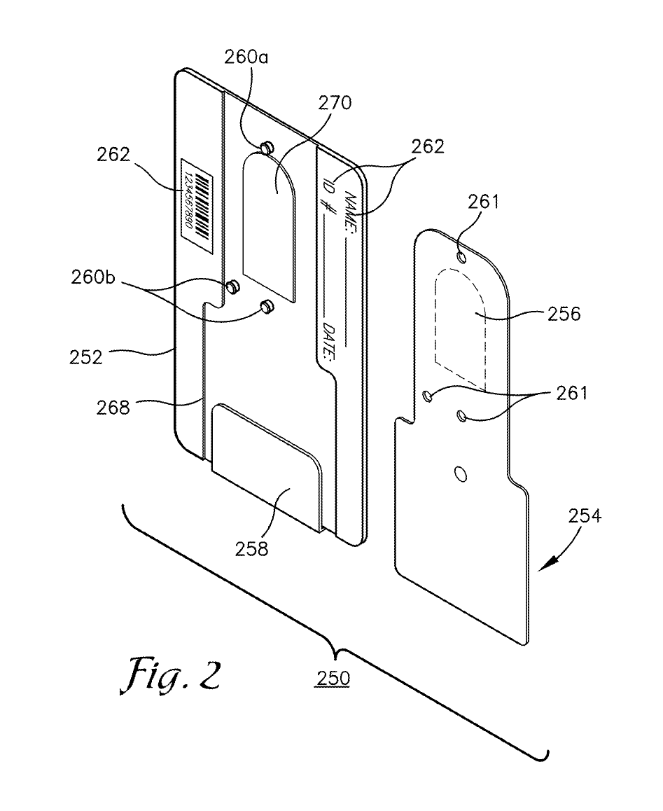

Referring now to FIG. 1 and FIG. 2, a first embodiment of a collection device with resident reagent is shown. The embodiment of FIG. 1 and FIG. 2 is comprised of the collection device embodiment 250 which comprises a storage holder 252 and a specimen collector 254 mounted therein. The specimen collector 254 is provided with a specimen collection absorbent 256 which is typically paper or cotton or polyester material suitable for receiving a biological specimen thereon.

Referring now to FIG. 2, it will be appreciated that during use, the specimen collector 254 is separated from the storage holder 252 and held by its lower edge by a user. The user then contacts the specimen collection absorbent 256 of the specimen collector 254 with the biological specimen to be collected. Once the specimen has been collected on the specimen collection absorbent 256 of the specimen collector 254, the specimen collector 254 can be repositioned onto the storage holder 252 by inserting the base of the specimen collector 254 into the pouch or flange area 258 which extends from the lower edge of the storage holder 252. The pouch 258 serves to capture the bottom edge of the specimen collector 254 to assist in registering the specimen collector 254 within the storage holder 252. This allows the specimen collector 254 to register with receiving tracks or indents 268 of the storage holder 252 to achieve alignment of, and bring into close contact the collection absorbent 256 of the specimen collector 254 with a reagent area 270 of the storage holder 252. It will be appreciated that typically a removable liner (not shown) is initially covering the reagent area 270 and that the removable liner is removed from the reagent area 270 to expose the pre-positioned reagents on the reagent area 270. In some embodiments, the removable liner is affixed to the storage holder 252 by an adhesive layer lining the storage holder 252 at the points of contact of the liner with the storage holder 252. When the removable liner is affixed to the storage holder 252 using an adhesive, the adhesive that is exposed by the removal of the removable liner remains on the storage holder 252 may be used to secure the specimen collector 254 to the storage holder 252, and to maintain the contact of the collection absorbent 256 with the reagent area 270.

In some embodiments, the storage holder 252 may further comprise registration pins 260a and 260b, and the specimen collector 254 may further comprise registration holes 261 on the specimen collector 254. In such embodiments, when the pouch 258 captures the bottom edge of the specimen collector 254, the pouch 258 provides vertical registration of the specimen collector 254 with the registration pins 260a, 260b and the registration holes 261 on the specimen collector 254. In this manner, accurate presentation of the specimen collector 254 with respect to the storage holder 252 is provided. Further, it will be appreciated that the registration pins 260a and 260b are in an asymmetrical formation. This permits only one orientation of the specimen collector 254 with respect to the storage holder 252. In this manner, the side of the specimen collector 254 which has received the specimen thereon will be correctly orientated to contact the reagent area 270 which is positioned on the storage holder 252 as seen in FIG. 2. Other methods of insuring proper orientation of the specimen collector 254 with respect to the storage holder 252 may also be envisioned. For instance, the registration pins 260a and 260b may be vertically aligned, but the vertically aligned registration holes and pins may be provided with an interior sloping side, thereby preventing incorrect orientation of the specimen collector 254 within the storage holder 252.

During use, the user first inserts the lower end of the specimen collector 254 into the pouch 258 to align the bottom edges of the storage holder 252 and the specimen collector 254. Then the user should observe the relationship of the storage holder 252 and the specimen collector 254 to determine that the registration pins 260a, 260b and the registration holes 261 on the specimen collector 254 are orientated for proper contact and registration within one another. If it is determined that the specimen collector 254 is properly orientated within the storage holder 252, the user then firmly presses the specimen collector 254 against the storage holder 252 to bring the collection absorbent 256 of the specimen collector 254 in contact with the reagent area 270 after first removing any protective liner from the reagent area 270. This then allows the reagents on the reagent area 270 to contact the specimen collected on the collection absorbent 256 and to provide delivery of the stabilizing effects of the reagents to the specimen collected on the collection absorbent 256.

Once contact between the reagent area 270 and the specimen collected on the collection absorbent 256 has been achieved, the entire collection and storage device 250 may be provided to a laboratory for analysis. The collection and storage device 250 may also be used with a cassette for long-term storage as described further below.

Still referring to FIGS. 1 and 2, an embodiment is shown wherein the storage holder 252 and the specimen collector 254 are provided with unique identification indicia 262 which may be in the form of bar codes and information regarding the sample and the individual from whom the sample was taken and the time and other identifying and informational indicia. As it will be recognized in the art, the indicia is used to identify and to track the location of the combination storage holder 252 and specimen collector 254 of the collection device 250 throughout the entire collection and investigation and analysis process, thereby providing a documented chain of custody.

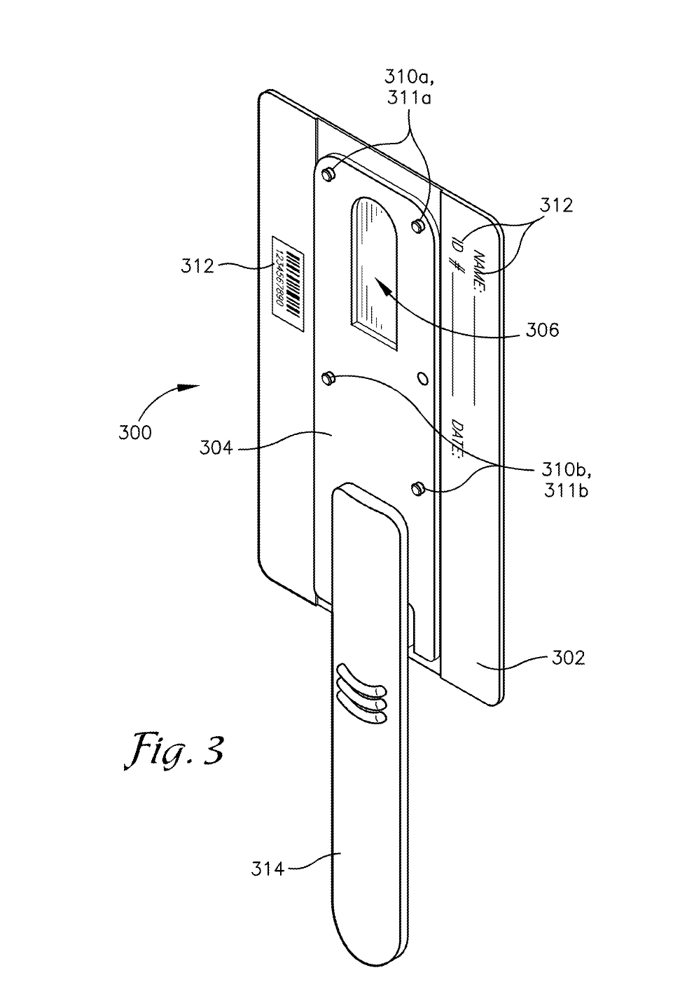

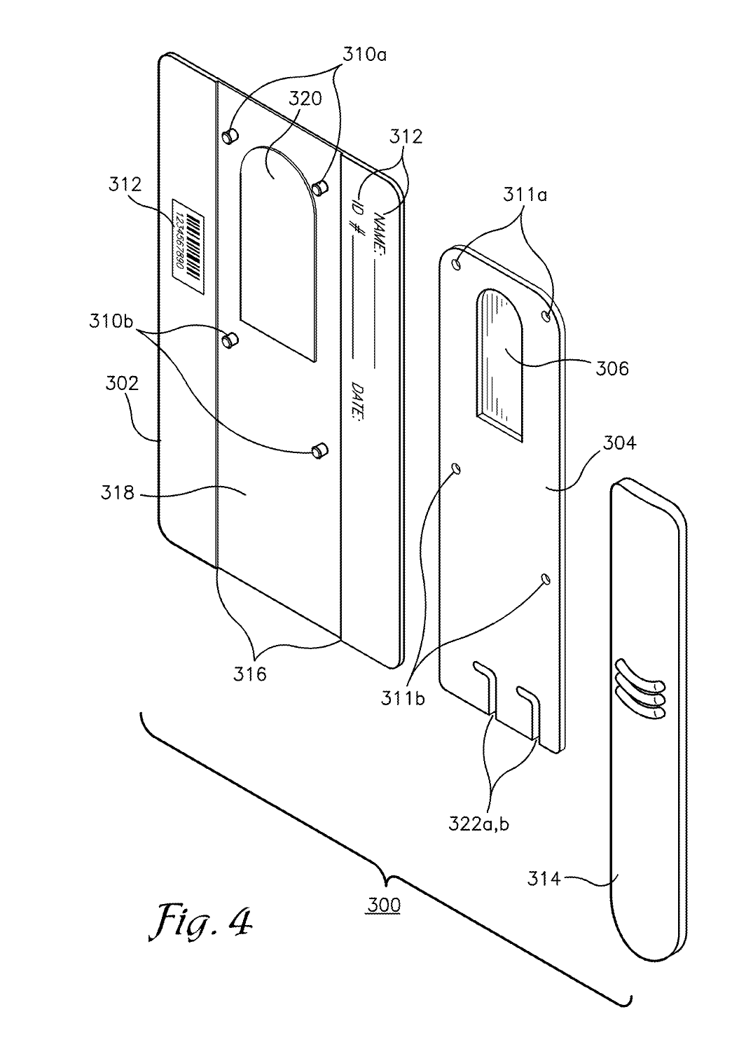

Referring now to FIG. 3 and FIG. 4, an alternate embodiment of a collection and storage device 300 is provided. In this embodiment, the collection and storage device 300 is equipped with a handle 314 which is attachable and detachable from the specimen collector 304. Such a handle 314 is of particular utility when the device is used by police officers who are often engaged with hostile individuals who may attempt to bite the officer while taking a specimen collection from the mouth. The addition of the handle 314 permits the officer to maintain a safe distance from the hostile individual while collecting the specimen. Subsequently, the handle 314 may be detached for shipping of the collection and storage device 300 to a laboratory for analysis. It will be observed that in FIG. 3 the handle 314 extends downwardly from the specimen collector 304.

Referring still to FIG. 3, the collection and storage device 300 is similarly structured to the collection and storage embodiment 250. A storage holder 302 is provided within which mounts the specimen collector 304 having collection area 306 thereon. The storage holder 302 is provided with registration pins 310a, 310b which are asymmetrically positioned. This permits only one orientation for the receiving of the specimen collector 304 thereon. This asymmetric positioning of the registration pins 310a, 310b with registration holes 311a, 311b (more easily observed in FIG. 4) on the specimen collector 304 insures that proper orientation of the specimen collector 304 with respect to the storage holder 302 will be achieved and that the side of the specimen collector 304 upon which a specimen is collected is properly orientated to contact the reagent area 320 (FIG. 4) on the storage holder 302. Other methods of insuring proper orientation of the specimen collector 304 with respect to the storage holder 302 may also be envisioned. For instance, the registration pins 310a, 310b and holes 311a, 311b may be vertically aligned, but the vertically aligned registration holes 311a, 311b and pins 310a, 310b may be provided with an interior sloping side, thereby preventing incorrect orientation of the specimen collector 304 within the storage holder 302 as described for the embodiment of the specimen collection device of FIGS. 1 and 2.

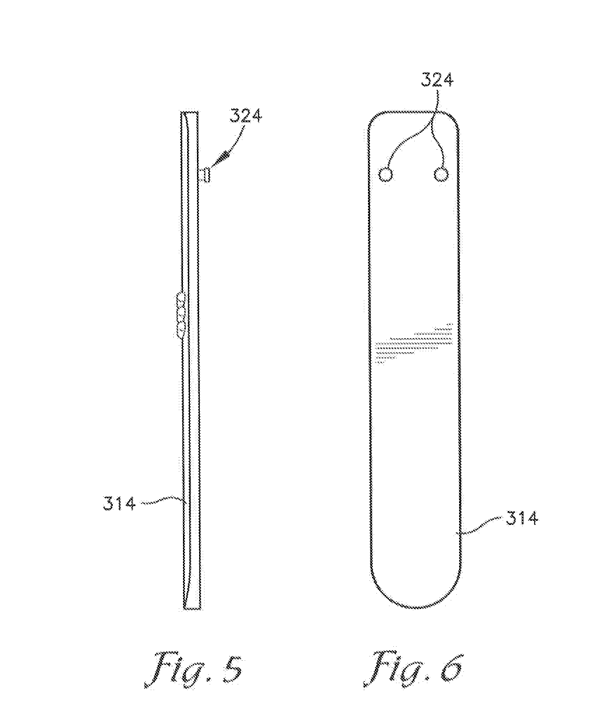

Referring now to FIG. 4, the collection device of FIG. 3 is shown in exploded view. The storage holder 302 is shown having a depression area 318 which is sized to receive the specimen collector 304 therein. Edge guides 316 are provided on either side of depression area 318 to further assist in the locating of the specimen collector 304 onto the storage holder 302. At the bottom edge of the specimen collector 304 are shown handle slots 322a, 322b which are adapted to receive therein handle pins 324 (see FIGS. 5 and 6) which extend from the handle 314. The two handle pins 324 which extend from the handle 314 align with the handle collection slots 322a, 322b and by providing an upward and sideways motion, the handle pins 324 lock into the handle slots 322a, 322b by a frictional fit. The insertion and removal of the handle 314 can be accomplished with the specimen collector 304 engaged with the storage holder 302, or it can be accomplished when the specimen collector 304 is separated from the storage holder 302. As previously mentioned, the advantage of the removal of the handle 314 is that the individual making a specimen collection can determine whether or not the use of the handle 314 will be beneficial for either safety purposes in dealing with a hostile individual during specimen collection, or for convenience in reaching a specimen which is in a difficult location and which might prevent the user's hand from effectively presenting the specimen collector 304 to the area or specimen to be collected. Another advantage is that the removal of the handle 314 allows for long-term storage of the collection and storage device 300 after the removal of the handle 314 in a storage cassette as described below.

Still referring to FIGS. 3 and 4, it will be recognized that, as with the embodiment of specimen collector 250, the storage holder 302 and the specimen collector 304 may be provided with unique identification indicia 312, which may be in the form of bar codes and information regarding the sample and the individual from whom the sample is taken and the time and other identifying and informational indicia.

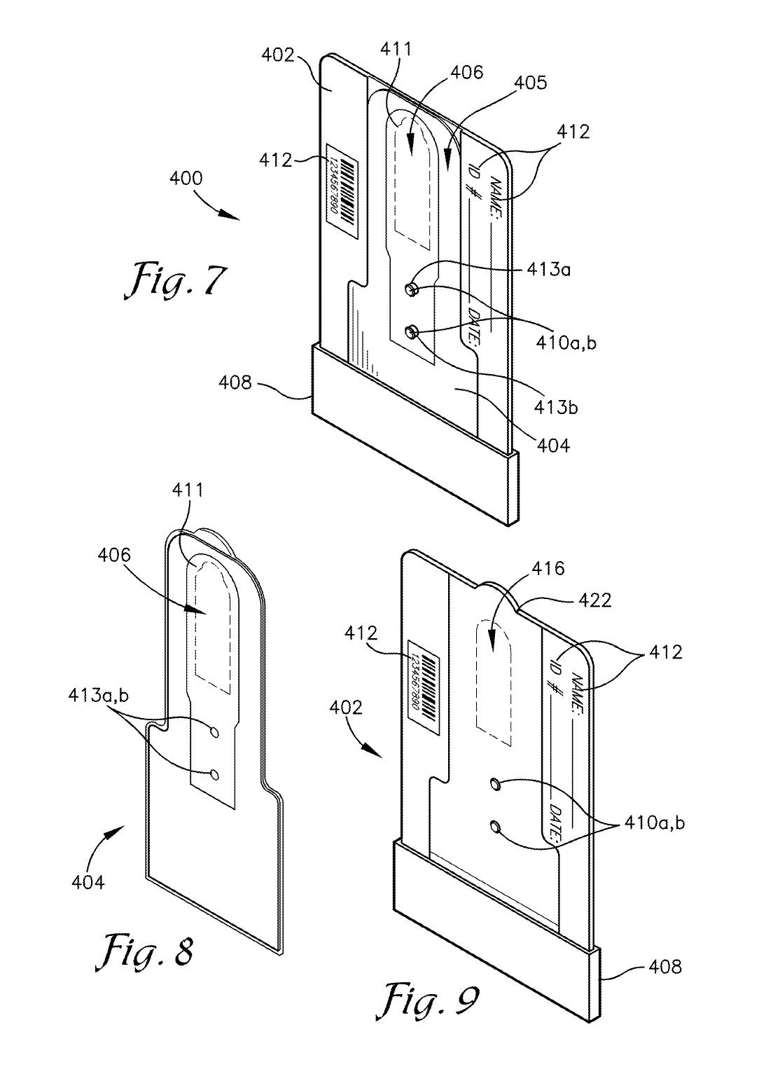

First referring to FIGS. 7, 8 and 9, an alternate collection and storage device will be described. In FIG. 7 the assembled device is shown. It is comprised of a collection and storage device 400 having a storage holder 402 which receives the specimen collector 404 therein. The specimen collector 404 is aligned with the storage holder 402 through the use of pouch 408 at the base of the storage holder 402 to provide correct alignment of the bottom of the specimen collector 404 with the bottom of the storage holder 402, thereby presenting proper positioning of registration pins 410a, 410b with registration holes 413a, 413b on the specimen collector 404. It will be appreciated by those skilled in the art that to achieve proper orientation of the vertically aligned registration holes and pins, the registration holes 413a, 413b on the specimen collector 404 may be provided with an interior sloping side, thereby preventing incorrect orientation of the specimen collector 404 within the storage holder 402 and insuring that the side of the specimen collector 404 upon which the specimen is placed is orientated toward the storage holder 402 and the reagents contained thereon as will be described hereinafter. Other methods of insuring proper orientation of the specimen collector 404 with respect to the storage holder 402 may also be envisioned. For instance, the registration pins 410a, 410b and registration holes 413a, 413b may be in asymmetrical formation to prevent incorrect orientation of the specimen collector 404 within the storage holder 402 as described for the embodiment of the specimen collection device of FIGS. 1 and 2.

Referring to FIG. 7, a removable cover 411 is placed over the surface of the specimen collector 404 which performs several functions. First, the removable cover 411 serves to protect the reverse side 407 of the collection area 406 from contamination, while also serving to cover the side of the registration holes 413a, 413b for which insertion of the registration pins 410a, 410b is not intended. The removable cover 411 ultimately is removed from the collection and storage device 400 when the device reaches a laboratory and it is desired to have access to the collection area 406 by the laboratory for punching of test disks from the collection area 406.

Referring now to FIG. 8 and FIG. 9, the specimen collector 404 is shown as separated from the storage holder 402. The specimen collector 404 in FIG. 8 is shown orientated to be placed into the storage holder 402 by first inserting the bottom edge of the specimen collector 404 into the pouch 408 to register the bottom of the specimen collector 404 with the bottom of the storage holder 402. In use, the storage holder 402 and the specimen collector 404 can either be presented to the user as an assembled unit protected by shrink wrap or as two individual pieces protected by shrink wrap. In either case, a removable liner 422 is provided to cover the reagent area 416 on the storage holder 402. The removable liner 422 is provided to assure that the reagents are not contaminated and that the reagents are not inadvertently contacted by the user or by the collection area 406 prior to collection of a specimen thereon. It will be appreciated that after a specimen has been collected on the collection area 406 of the specimen collector 404, the removable liner 422 is removed prior to the insertion of the specimen collector 404 into the storage holder 402. As with the embodiment of specimen collector 250, the storage holder 402 and the specimen collector 404 may also be provided with unique identification indicia 412.

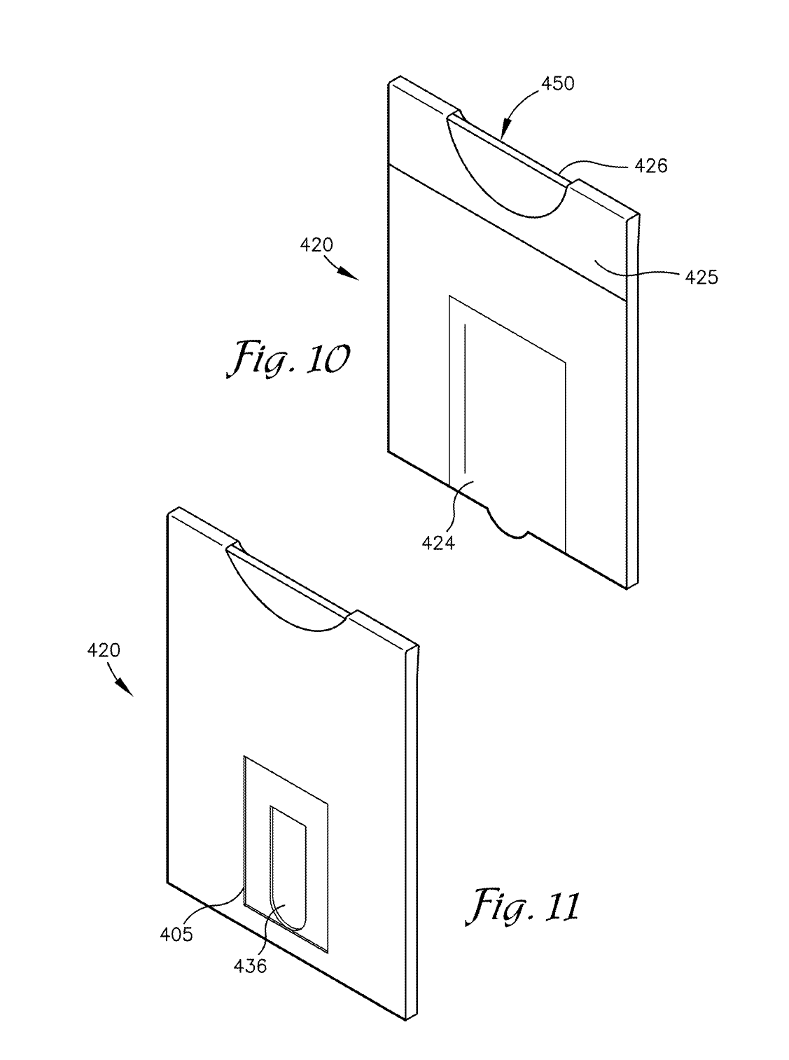

Referring now to FIG. 10 and FIG. 11, a cassette 420 is shown which is optionally used for long-term storage of any of the collection and storage devices described above. After a sample has been collected on a specimen collector and the specimen collector is inserted into the storage holder, the combination 450 of the specimen collector and the storage holder may be inserted into the cassette 420 for shipment and/or storage and/or archival purposes and/or extraction of a specimen portion from a specimen collection area. All of these benefits and functions may be achieved without ever again removing the combination 450 of the specimen collector and the storage holder from the cassette 420. This is possible because the cassette 420 is provided with a specimen window 405 (shown in FIG. 11) which permits both visual inspection of the collection area 436 and/or the reagent area (hidden in FIGS. 10 and 11) of the combination 450, depending upon which side the cassette 420 is viewed. The specimen window 405 also permits a lab analyst to remove a portion of the collection area 436. This removal of a specimen portion from the collection area 436 is typically accomplished by punching small disc portions out of the collection area 436 and then subjecting the discs to various forms of analysis as desired by the analyst. The cassette 420 is initially provided with a cassette liner 424 covering the specimen window 405 on either side of the cassette 420. When it is desired at the laboratory to remove a portion of a collection area 436 for testing and analysis, the cassette liners 424 on both the front and back of the cassette 420 are removed, thereby exposing the collection area 436. This permits punching of a test portion from the collection area 436. Such exposure of the collection area 436 while the combination 450 of the specimen collector and the storage holder is in the cassette 420 is shown in FIG. 11.

As it will be recognized in the art, the cassette 420 may further be provided with unique identification indicia (not shown), which may be in the form of a bar code or other suitable unique indicia. The indicia is used to identify and to track the location of the cassette 420 and the combination 450 of the specimen collector and the storage holder throughout the entire collection and investigation and analysis process, thereby providing a documented chain of custody. It will also be appreciated that a closure flap 425 on the cassette 420 is used to seal the collector and holder combination 450 into the cassette 420. The closure flap 425 is provided with a flap window 426. When the collector and holder combination 450 comprises identification indicia, the indicia on the collector and holder combination 450 may be observed through the flap window 426. Thus, in the flap window 426, the indicia of the collector and holder combination 450 can be read through the window 426 and can be separately confirmed as to its location and presence apart from the identification indicia of the cassette 420. It will be appreciated that identification indicia on the cassette 420 and indicia on the collector and holder combination 450 may be identical or they may be different. The ability to read either or both of the identification indicia eliminates the need for the user on the crime scene to be concerned with matching collector identification indicia with the identification indicia on the cassette 420. While it may be considered preferable to have the two indicia match, the alternative exists for them to be different and to be separately accounted for in the device of the present invention.

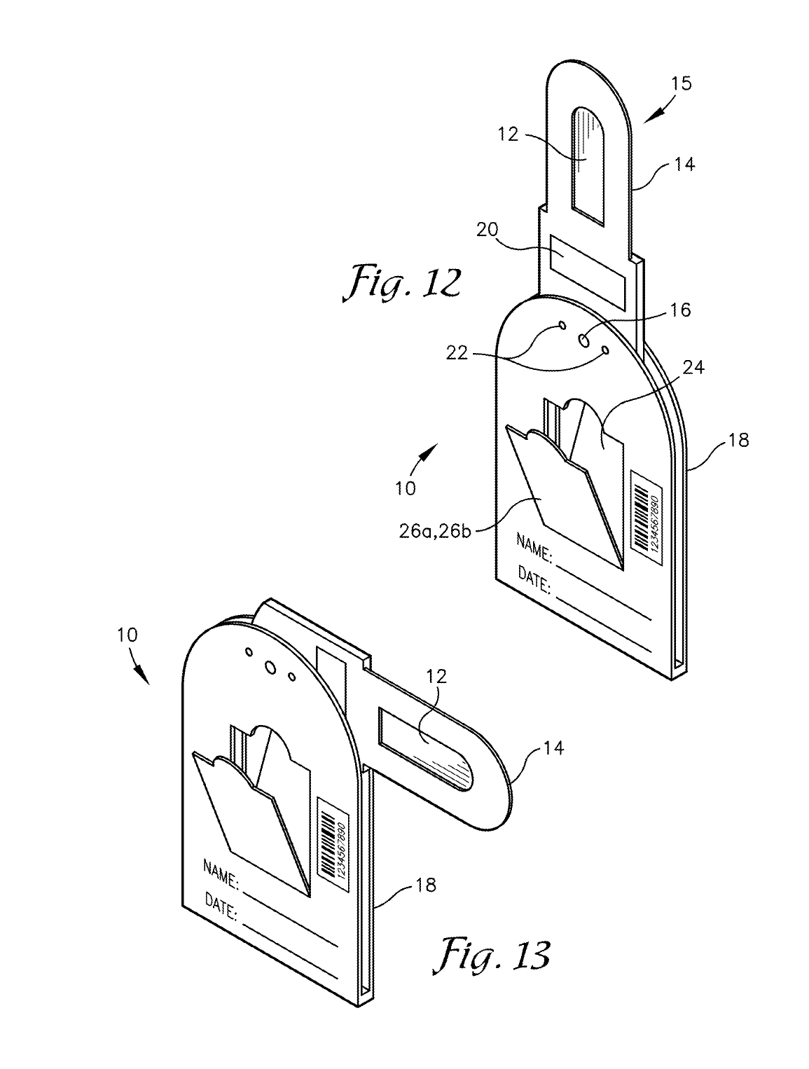

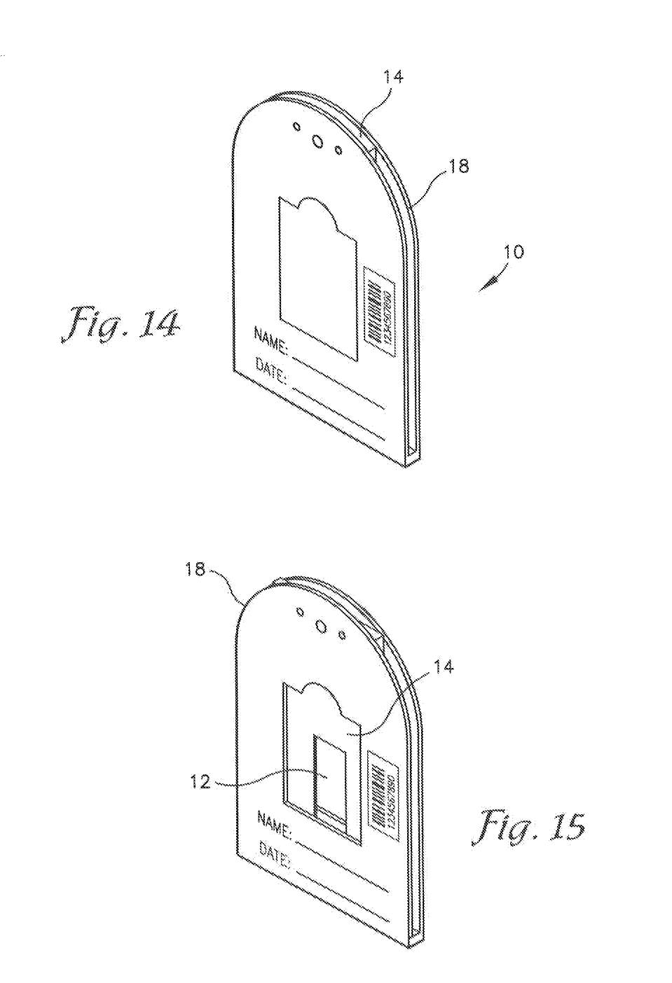

Referring now to FIGS. 12-15, the embodiment 10 shows a specimen collector 15 which extends from a protective storage holder 18, the specimen collector 15 being rotatable between an open exposed position shown in FIG. 12 and a closed protected position shown in FIG. 14. The rotation of the specimen collector 15 into the storage holder 18 is shown in FIG. 13. The specimen collector 15 is comprised of a collector paper 12 mounted on a rotatable collector frame 14, which rotates on a pivot 16 to place the frame 14 either inside or outside of the holder 18. When the specimen collector 15 is rotated for placement inside of the holder 18, it may be secured in that position by use of peel and seal adhesive 20 or by the insertion of pegs (not shown) into peg closure holes 22, the objective being to securely hold the frame 14 and collector paper 12 inside of the holder 18 once a specimen is collected on the paper 12.

It will be appreciated that the collection and storage device 10 may be initially provided in a shrink wrap or similar packaging in the open position shown in FIG. 12 before initial use to prevent contact between the reagent and the specimen collection area before specimen collection. After a biological specimen has been collected on the paper 12 and the specimen collector 15 has been rotated to insert the collector paper 12 within the holder 18, reagents on a translucent layer 24, which layer is covered by removable flap 26, can be transferred onto the collector paper 12 to prevent changes in the sample and to prevent deterioration of the sample. Once the collector paper 12 is within the holder 18 and it is desired to apply the reagents to the collector paper 12, the user firmly presses against the removable flap 26 to transfer the reagents onto the collector paper 12. It will be appreciated that the reagents on the translucent layer 24 may be located on both the back and front of the holder 18. This is a matter of having a front and back removable flap in 26a, 26b, and a front and back positioned reagent coated protective translucent layer 24 on the holder 18. It will be understood by those skilled in the art that as a biological specimen could be applied to either side of the collector paper 12, having reagents 24 on both sides of the holder 18 is preferred. Referring now to FIGS. 13 and 14, the rotation of the specimen collector 15 is shown in FIG. 13 as it is rotated into a position within a side of the holder 18, as is shown in FIG. 14. Referring now to FIG. 15, the specimen collector 15 is shown contained within the holder 18 and the removable flap 26a (and 26b if present) have been removed to provide access to the specimen collected on the collector paper 12. With the flaps 26a, 26b removed, samples may be punched from the collector paper 12 to allow analysis of the specimen to take place. The advantage of the structure of the embodiment of FIGS. 12-15 is that a specimen may be collected onto the paper 12, the paper then secured within the holder 18, the reagents applied to prevent deterioration of the sample and, subsequently, samples of the specimen taken directly from the evidence collector device 10 without opening the device or disturbing the device and thereby damaging the chain of evidence custody that has been established. In some embodiments, the collection and storage device 10 may further be used with a cassette for long-term storage as described above.

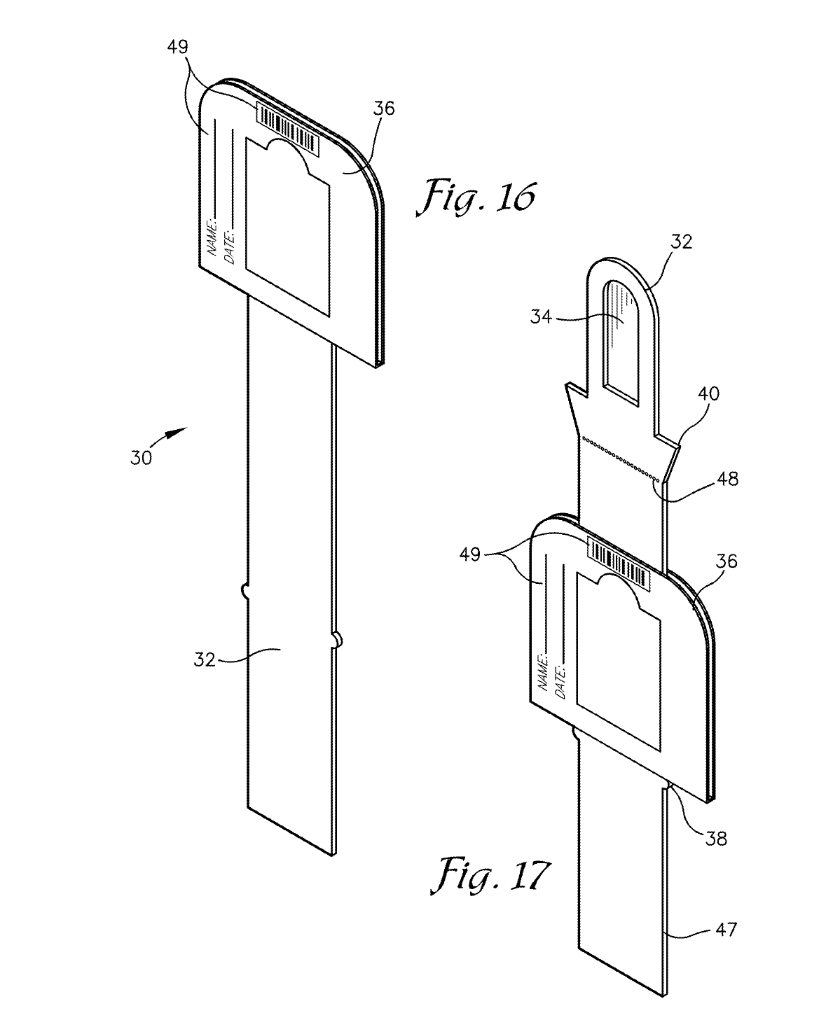

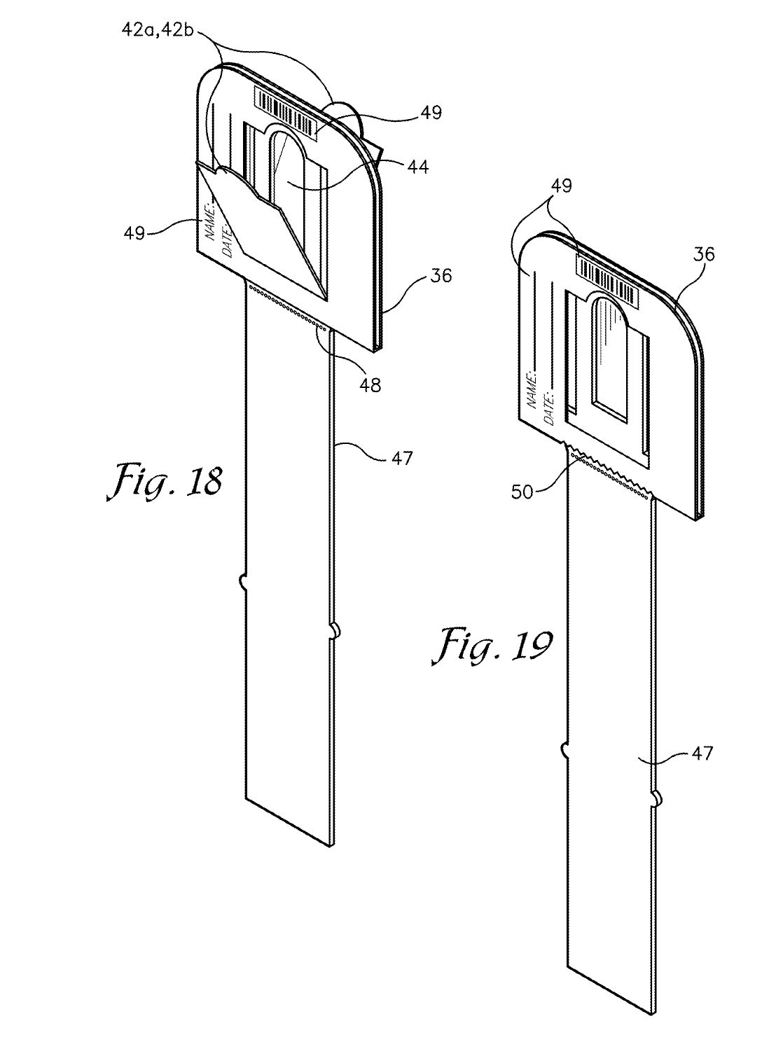

Referring now to FIGS. 16-19, another embodiment of the collector with reagents is shown. In this embodiment, the collector 30 is comprised of an elongated support frame 32 having the collector absorbent or collector paper 34 mounted on one end (FIG. 17) and the protective holder or shell 36 being slideable along the length of support frame 32 to either expose the absorbent collector paper 34 or to enclose it within the holder 36. Many of the features for the embodiment of FIGS. 16-19 are similar to those of FIGS. 12-15. However, additional features are present in the embodiment of FIGS. 16-19. When the user desires to expose the absorbent paper 34 of the embodiment of FIGS. 16-19 for use, the user slides the storage holder 36 downwardly on support frame 32 until the storage holder is stopped by stops 38 mounted on either side of the support frame 32. It will also be appreciated that the collection and storage device 30 may initially be provided in a shrink wrap or similar packaging in the downward position shown in FIG. 17 before initial use to prevent contact between the reagent and the specimen collection area before specimen collection. When the absorbent collector paper 34 is exposed with the storage holder 36 in the downward position, the user may then apply the biological specimen to the collector paper 34. Once the specimen has been collected on absorbent paper 34, the user will slide the holder 36 upwardly to cover the paper 34 and to contain the paper 34 within the holder 36. The upward movement of the holder 36 is halted by stops 40 mounted on either side of the support frame 32. Referring now to FIG. 18, it can be seen that the holder 36 is provided with a removable flap on front and back 42a, 42b, under which is a chemical reagent layer 44. It will be appreciated by those skilled in the art that the reagent layer 44 may be an actual separate layer as is shown in FIG. 18, or alternatively, the reagent layer 44 may be applied to the inside face of removable flaps 42a, 42b forming a thin chemical layer on the inside of the removable flaps 42a, 42b and thus avoiding an additional layer 44. The operation of the chemical layer in embodiments of FIGS. 16-19 is similar to that described for FIGS. 12-15. Once the specimen on the collector paper 34 has been covered by the holder 36, the user can firmly press against the removable flaps 42a, 42b to bring the reagent layer 44 into contact with the specimen on the collector paper 34 to thereby preserve the specimen and to prevent deterioration of the specimen. After application of the reagents to the collected specimen on the collector paper 34 has been accomplished, the user may then, as desired, detach the handle portion 47 of the support frame 32 by use of perforations 48 which are located below the stop 40 of the support frame 32. Referring now to FIG. 19, further assistance in removing the handle 47 from the device may be obtained by the addition of striations 50 to the edge of the holder 36. In FIG. 19, it is also shown that removable flaps 42a, 42b have been removed by cutting or tearing from the holder 36, and the absorbent collector paper 34 is exposed to allow removal of a specimen from the collector paper 34 by whatever technique the user may wish to employ.

The collection and storage device 30 may also be used with a cassette for long-term storage as described above. Additionally, it will be recognized that, as with the embodiment of specimen collectors previously described herein, the holder 36 may be provided with unique identification indicia 49, which may be in the form of bar codes and information regarding the sample, and the individual from whom the sample is taken and the time and other identifying and informational indicia.

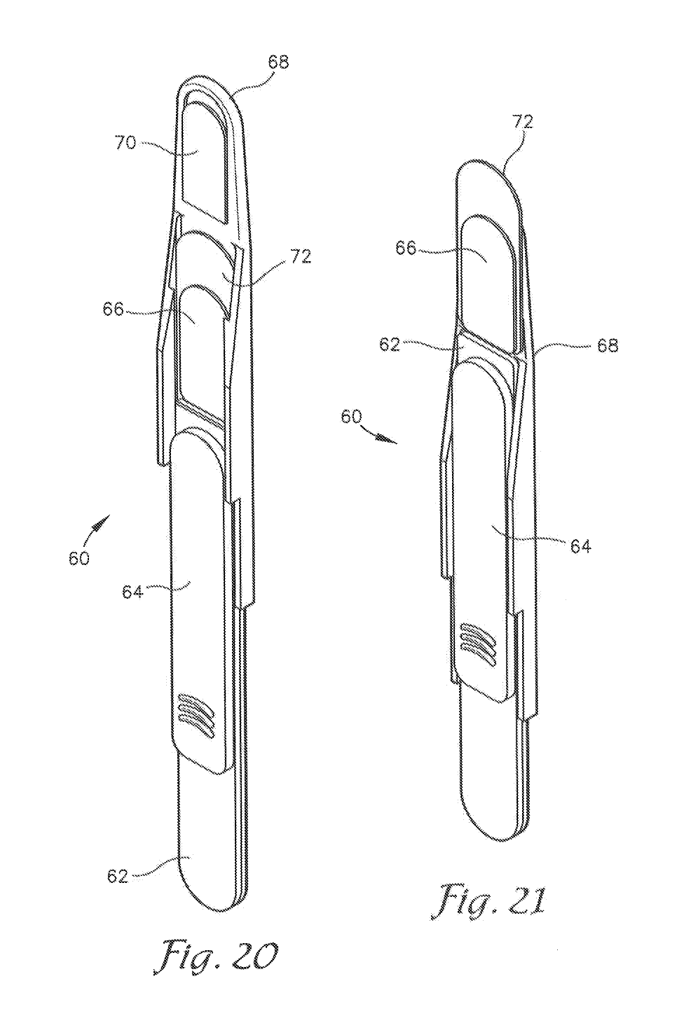

Referring now to FIGS. 20-24, another embodiment of a specimen collector having a reagent-lined cassette is shown. In FIG. 20, device 60 is comprised of a handle 62 having a slider or sliding cover 64 mounted thereon. The slider 64 is capable of moving forwardly and backwardly on the handle 62 to either expose or to cover the entire collector paper 66. At an end of the handle 62 is a support 68 having a reagent pad 70 mounted thereon. As will be described hereinafter, the device 60 is capable of moving the handle 62 forwardly and backwardly on the support 68 to move the collector paper 66 upwardly and onto the support 68 where a specimen can be collected on the collector paper 66. During specimen collection, when the collector paper 66 is advanced upwardly so that it is in position over the support 68, the collector paper 66 is protected from the reagent pad 70 by a removable barrier sheet 72. The cooperation of these elements will be further described with reference to FIGS. 21-24.

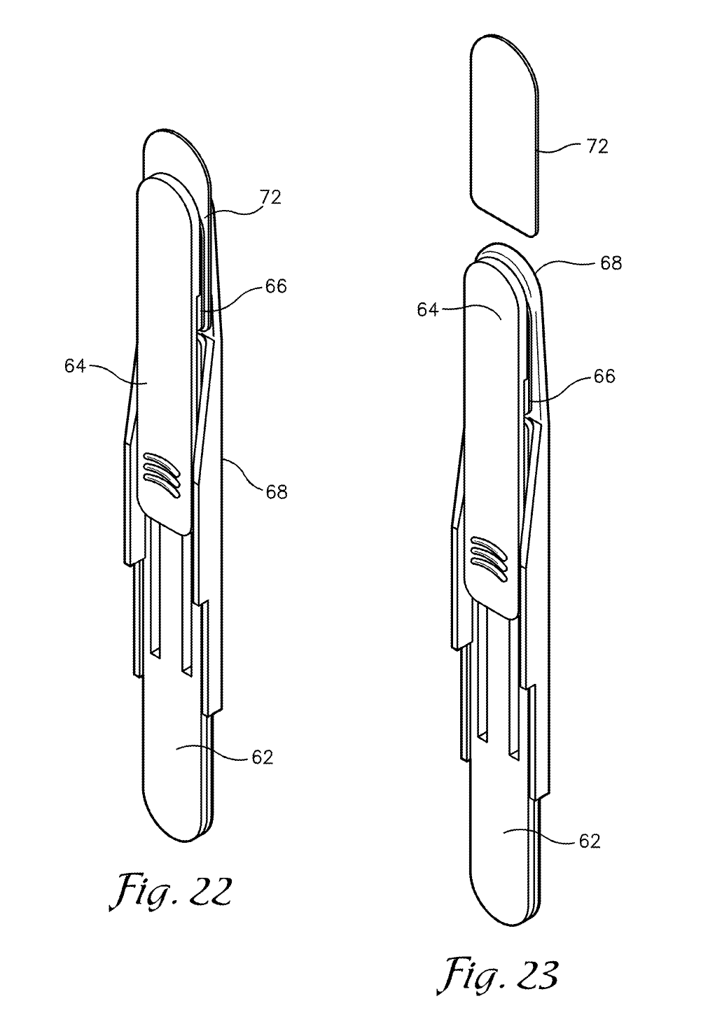

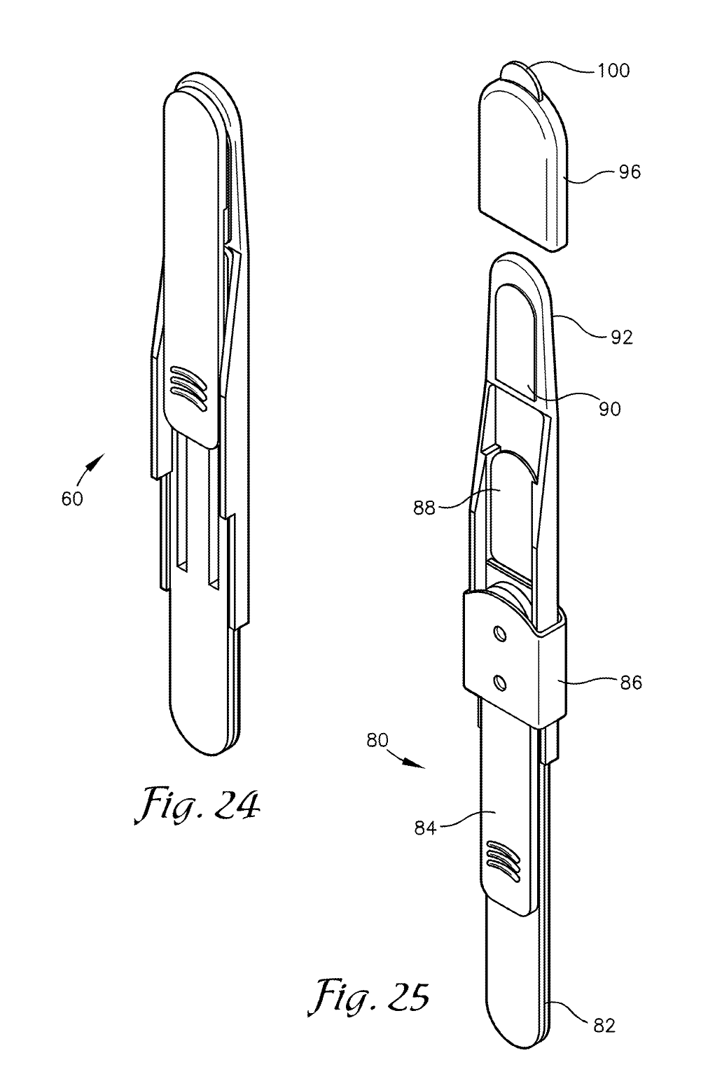

In FIG. 21, the handle 62 has been advanced within the support 68 to position the collector paper 66 and the removable barrier sheet 72 over the reagent pad 70 and the support 68. This positions the collector paper 66 in position for collection of a specimen thereon. The presence of the removable barrier 72 protects the specimen collection area from contact with the reagent pad 70 mounted on the support 68. With the device configured as is shown in FIG. 21, the device 60 can be manipulated to contact a biological specimen with the collector paper 66 and thereby collect the biological specimen thereon. Once the specimen has been collected onto the collector paper 66, the slider 64 can be further advanced to cover the collector paper 66 and protect it from further specimen collection or contamination as is shown in FIG. 22. Once the slider 64 is covering the collector paper 66, the user can remove the removable barrier sheet 72 as is shown in FIG. 23. The removable barrier sheet 72 is held in place by a perforation line or line of weakness which allows it to be torn free from its connection within the device 60 to thereby place the collector paper 66 and the reagent 70 on the support 68 into close position if not contact. To then transfer the reagents on the reagent pad 70 to the collector paper 66, the user presses firmly on the end of the slider 64 which is covering the paper 66 to press the paper 66 against the reagent pad 70, thereby transferring the reagents from the pad 70 onto the paper 66. Once the transfer has been accomplished, the slider 64 can be locked into place by the pressure of the user pressing the slider 64 against the support 68, which will engage a ball and detent or other locking structure (not shown) to secure the slider 64 in contact with the support 68 as is shown in FIG. 24.

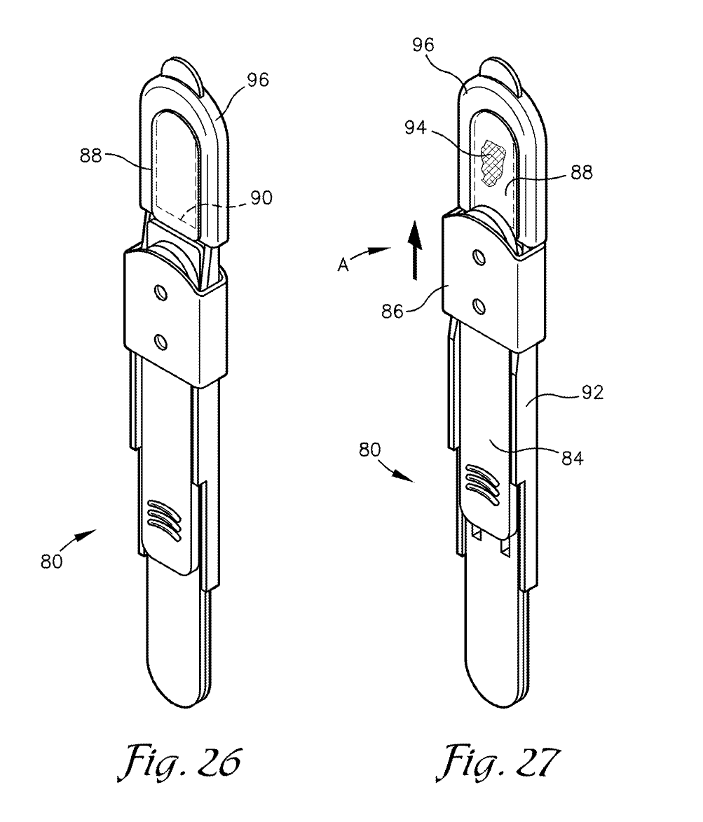

Referring now to FIGS. 25-28, another embodiment will be described. This embodiment is similar to the embodiment of FIGS. 20-24, however distinctions are provided in terms of a protective cap and operations of a slider cover. Device 80 is provided with a handle base 82 having a slider cover 84 mounted thereon and a protective cover 86 connected to the slider cover 84, the protective cover 86 providing pressure to make contact between the sample and the reagent layer when the slider cover 84 is shifted upwardly to position the sample collector paper 88 over reagent 90 on support 92. As previously described and shown in FIG. 21 for the previously described embodiment, the slider 84 can be moved upwardly to place the sample collector paper 88 in position for sample collection. This position is shown in FIG. 26 wherein the collector paper 88 is slid into position on top of a protective cap 96, which is placed over the support 92 to cover the reagent pad 90 to prevent contact between the sample collector paper 88 and the reagent pad 90, and also to prevent any contact between the reagent pad 90 and a surface from which a specimen is being collected. Such a specimen could be inside of a subject's mouth or it could be any surface on which a biological specimen resides. The presence of the protective cap 96 assures that the reagent pad 90 will not contact surfaces for which contact is not intended. Still referring to FIG. 26, the device 80 is shown in position for collection of the specimen onto the collector paper 88. This is accomplished by holding the device 80 in a hand and pressing the collector paper 88 against a surface where a specimen is to be collected. Once the specimen has been collected, the collector paper 88 with the sample thereon can be exposed to the reagent pad 90 and the slider 84 locked into place with pressure provided to press the paper 88 against the reagent 90 as will be described hereinafter.

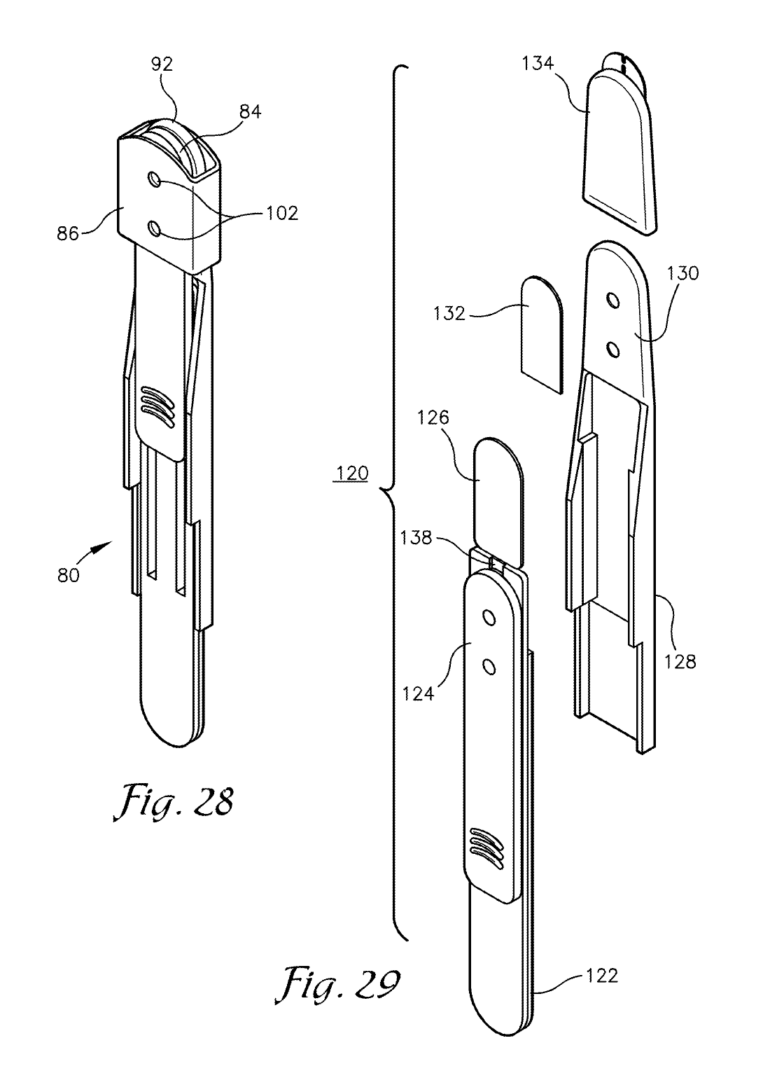

Referring now to FIG. 27, a specimen 94 has been collected onto the collector paper 88 and the user has moved the slider 84 upwardly to bring the protective cover 86 into contact with the lower end of the protective cap 96. As the slider 84 is pressed upwardly in the direction of arrow A (this may be better appreciated by referring to FIG. 25 showing an exploded view), continued pressure by the protective cover 86 against the lower end of the protective cap 96 exerted by the upward movement of the slider 84 separates the protective cap 96 from the support 92, thereby exposing the reagent pad 90. In FIG. 28, the device 80 is shown with protective cap 96 (not shown in FIG. 28) separated from the support 92 and the reagent pad 90 (hidden in FIG. 28), exposing the reagent pad 90 to contact with the sample collector paper 88 upon moving the slider cover 84 upwardly in the direction of arrow A of FIG. 27 to position the sample collector paper 88 over the reagent pad 90. Referring now to FIG. 28, after a specimen has been collected on the collector paper 88 (hidden in FIG. 28) and the protective cover 86 has been pressed upwardly using the slider 84 to place the paper 88 over the reagent pad 90, contact between the paper 88 and the reagent pad 90 can be securely effected by continuing to press the protective cover 86 upwardly and over the end of the slider 84 to secure the end of the slider cover 84 over the sample paper 88 where it causes pressure to be delivered against the paper 88, thereby pressing the paper 88 against the stabilizing reagent pad 90 (hidden in FIG. 28) to transfer the reagents on the reagent pad 90 onto the sample paper 88, thereby providing the stabilization of the collected specimen 94 (FIG. 27) on the paper 88. It should be noted that as an alternative, a pull tab 100 is provided on the end of the protective cap 96 (FIG. 25) which allows the user to remove the protective cap 96 from the stabilizing reagent pad 90 if the upward pressure on the slider 84 moving the protective cover 86 upwardly is insufficient to remove the protective cap 96 from covering the support 92 and stabilizing the reagent pad 90. In FIG. 28, ventilation holes 102 are shown on the protective cover 86 which allow passage of air between the outside environment and the collector paper 88 to assist in drawing of the specimen on the collector paper 88. Referring now to FIG. 28, the final configuration between the slider 84 and the support 92 and the protective cover 86 is shown with the ventilation holes 102 which extend through protective cover 86 to allow air to contact the sample collector paper 88.

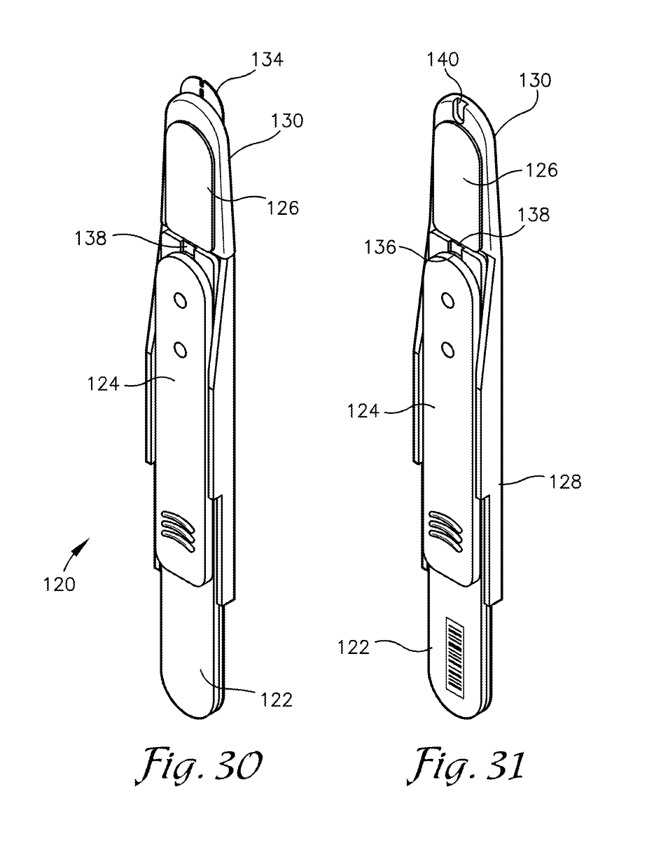

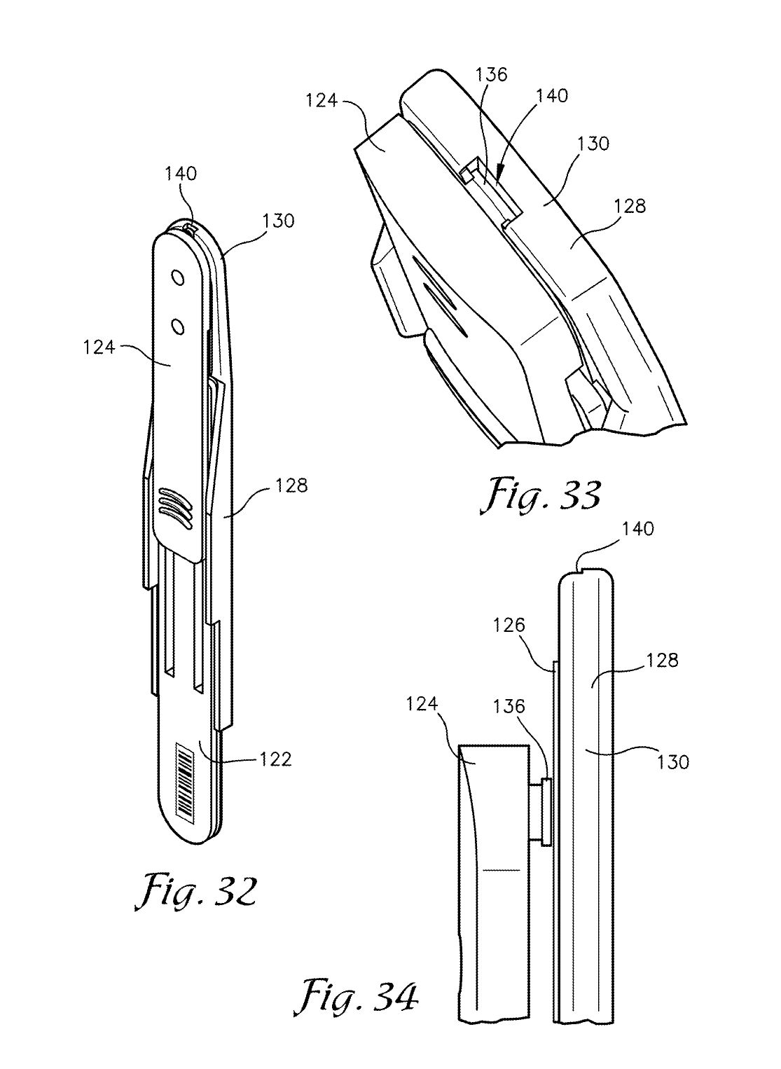

FIGS. 29-34 show yet another embodiment. In FIG. 29, embodiment 120 is shown, which is comprised of a handle 122 having a slider 124 mounted thereon and collector paper 126 extending from the handle 122. The handle 122, the slider 124, and the collector paper 126 are mounted onto a support 128 having a support region 130 thereon. Reagent pad 132 is mounted on the support region 130 and is protected by a shrink wrap protective cap or other protective cap 134 until such time as it is desired to expose the reagent pad 132 for contact with the specimen which has been collected on the collector paper 126. Referring now to FIG. 30, the device 120 is shown with the collector paper 126 in position for collection of a specimen thereon and extending over the protective cap 134 and support region 130. It will be appreciated that the protective cap 134 is covering the reagent pad 132 (hidden in FIGS. 30-34), and therefore the device 120 may be used for collection of a specimen thereon without concern about the reagents of the reagent pad 132 contacting the collector paper 126. Once a specimen has been collected on the collector paper 126, the cap 134 can be removed and the slider 124 moved upwardly to cover the collector paper 126. Referring to FIG. 31, it may be seen that the slider 124 is provided with a plug or projection 136 at the end of the slider 124 which is initially interconnected with a receptacle 138 on the handle 122 to interconnect the slider 124 with the handle 122, the receptacle 138 being of complimentary design to receive the projection 136 in the frictional fit. On movement of the slider upwardly such that the collector paper 126 is covered, the slider 124 can be released from the receptacle 138 and the projection 136 can then be fitted into a support receptacle 140 to secure the slider 124 against the support 128 to prevent further movement of the slider 124 and to lock the slider 124 into a protective covering position over the collector paper 126. FIG. 32 shows the slider 124 locked into position interconnected with the support 128 where it presses the collector paper 126 against the reagent pad 132 to transfer reagents from the pad 132 onto the collector paper 126. FIG. 33 shows a top perspective view of the interconnection between the projection on the slider 124 and the support receptacle 140 on the support 128 and showing the slider 124 retracted after being advanced to press the collector paper 126 against the reagent pad 132. FIG. 34 is a side view of the projection 136 prior to its insertion into the support receptacle 140 on the support 128, showing close passage of the projection 136 across the surface of the paper 126 prior to insertion of the projection 136 into the receptacle or notch 140 of the support 128.

In FIGS. 29-32, ventilation holes 139 are shown on the slider 124 which allow passage of air between the outside environment and the collector paper 126 to assist in drawing of the specimen and the stabilizing composition on the collector paper 126. Ventilation holes may also be provided on the support to further assist in ventilation.

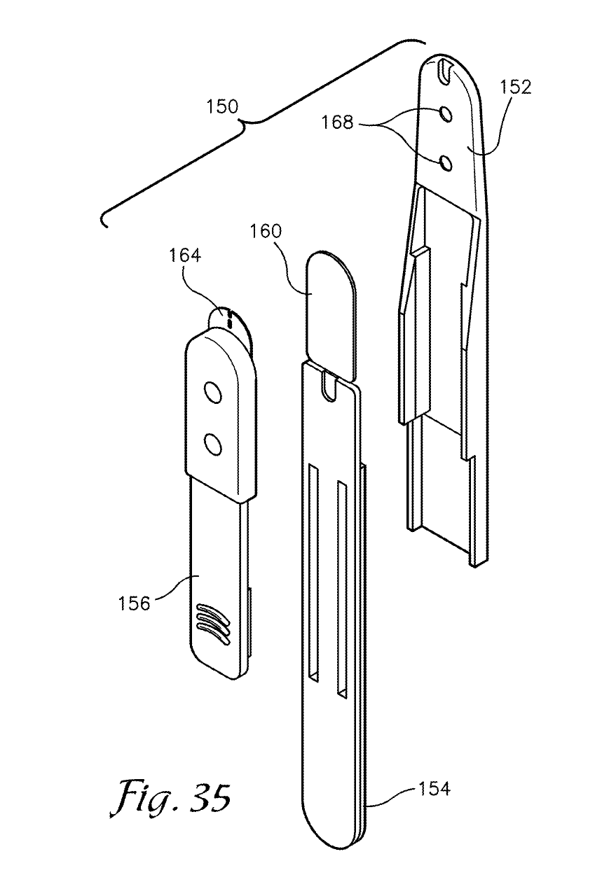

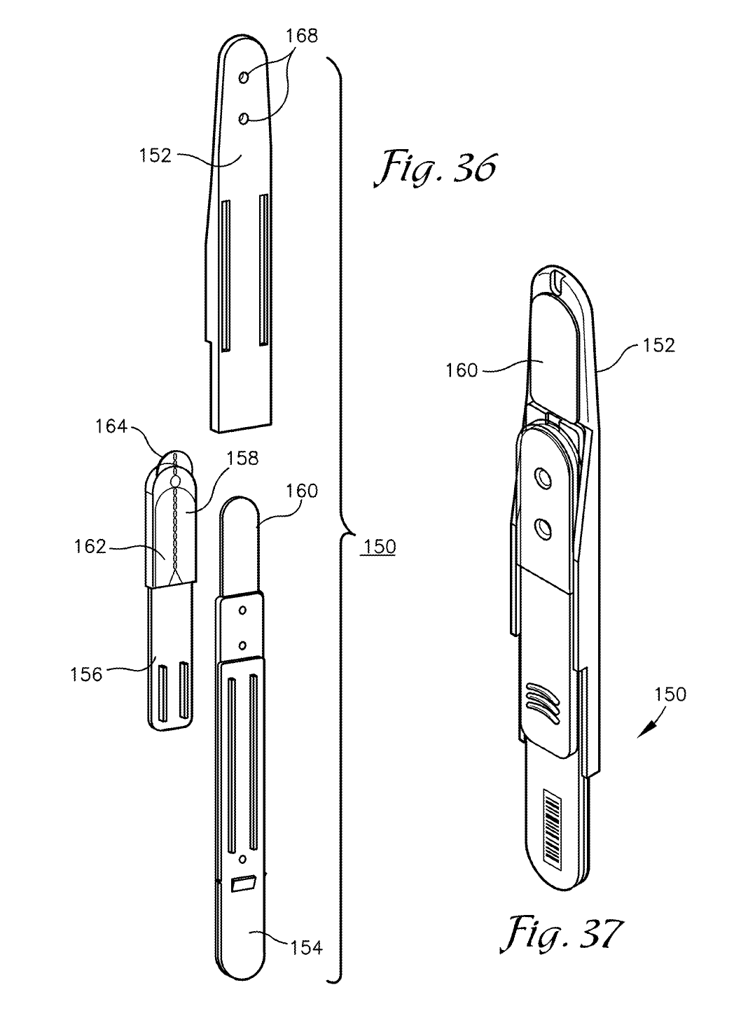

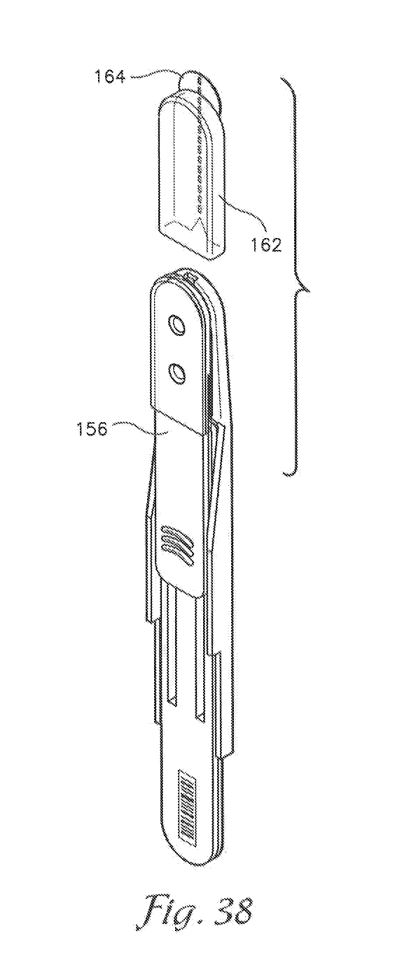

Referring now to FIG. 35, collector 150 is shown in exploded view. The collector 150 comprises a support 152 into which connects a handle 154 with a slider 156 connected thereto. A collector paper 160 extends from the handle 154. In embodiment of FIGS. 35-38, the reagent pad 158 is located on the slider 156. Referring now to FIG. 36, a reagent pad 158 is located on the slider 156 and a shrink wrap barrier 162 covers the reagent pad 158 with tab 164 extending from shrink wrap barrier 162 to permit removal of the shrink wrap barrier 162 from the reagent pad 158 by a user when it is desired to apply the reagents to collector paper 160. In FIG. 37, the collector 150 is shown configured for collecting a specimen with the collector paper 160 being in contact with the support 152 to reinforce the paper 160 as it is applied to an evidence specimen or biological specimen. In FIG. 38, after a specimen has been applied to the collector paper 160, the slider 156 may be pressed upwardly to place the slider 156 in position over the collector paper 160 and to position the reagent pad 158 (hidden in FIG. 38) over the collector paper 160. Once this arrangement has been established, the user can pull on shrink wrap tab 164 to remove the shrink wrap barrier 162 and to expose the reagent pad 158 to the collector paper 160 (hidden in FIG. 38). The pressing of the slider 156 against the support 152 and the collector paper 160 serves to transfer the reagents from the reagent pad 158 onto the paper 160. Once this has been accomplished, the slider 156 can be locked into position to protect the collector paper 160. This is accomplished using a plug and receptacle in much the same manner as described for the embodiment of FIGS. 29-34.

In FIGS. 35-38, ventilation holes 168 are shown on the support 152, which allow passage of air between the outside environment and the collector paper 160 to assist in drawing of the specimen and the stabilizing composition on the collector paper 126.

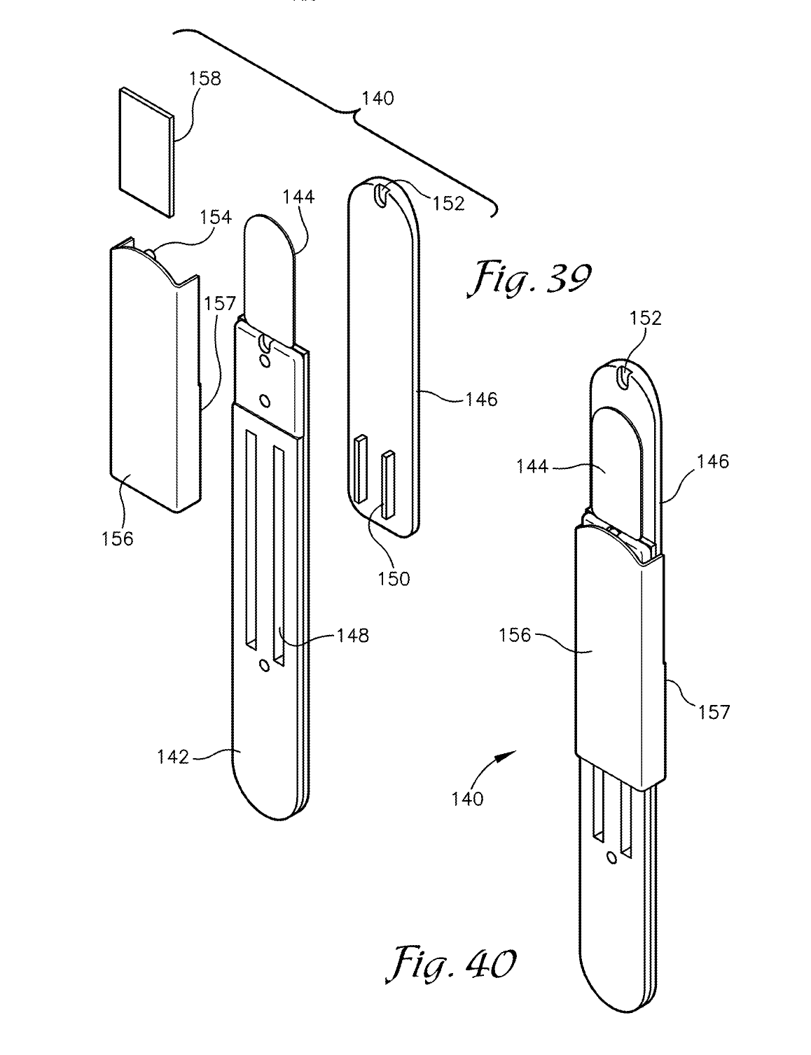

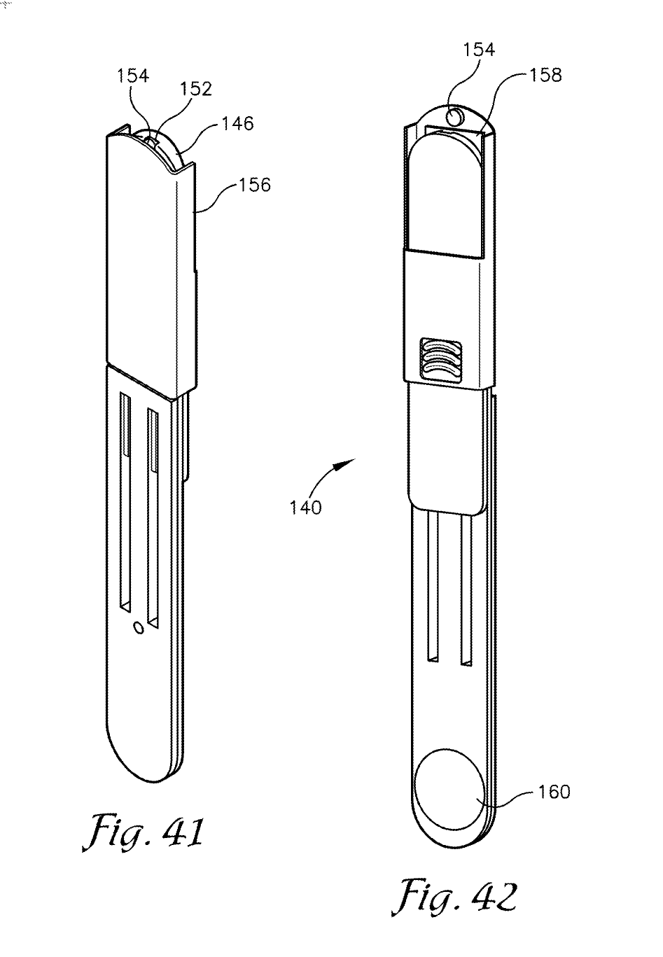

Referring now to FIGS. 39-42, embodiment 140 is shown. In FIG. 39, embodiment 140 is shown in exploded view showing handle 142 having collector paper 144 extending therefrom. A slider 146 connects to handle 142 and slides within tracks 148, which tracks 148 receive rails 150 of the slider 146. The slider 146 is equipped on its end with a snap-in void or detent 152, which is mateable with a projection 154 extending from a reagent pad cap 156 which slideably connects with handle 142 and slider 146 through lateral projections 157 extending inwardly from the sides of reagent pad cap 156. Reagent pad 158 (hidden in FIGS. 38-41, shown in FIGS. 38 and 42 mounts on an inside surface of the reagent pad cap 156 so that the reagent pad 158 may be brought into contact with the collector paper 144 upon the sliding of the reagent pad cap 156 upwardly along the handle 142 and the slider 146 to cover the collector paper 144. In FIG. 40, the device 140 is shown in configuration for collection of a specimen onto the collector paper 144 where the collector paper 144 is supported during the collection process by the slider 146. After the collection of a specimen on the collector paper 144, the reagent pad cap 156 may be pressed upwardly to cover the collector paper 144 with the reagent pad cap 156 and to bring the reagent pad 158 on an inside surface of the reagent pad cap 156 into contact with the collector paper 144. It will be appreciated once again that the reagent pad cap 156 can be constructed to a particular thickness such that when it is pressed upwardly, it causes compression between the reagent pad cap 156 and the slider 146 so as to provide compressive contact between the reagent pad 158 and the collector paper 144 to transfer reagents from the reagent pad 158 onto the collector paper 144. Referring now to FIG. 41, it will be appreciated that the reagent pad cap 156 is provided with projection 154, which connects into snap-in mechanism or detent mechanism 152 as described for the embodiment of FIGS. 29-34 to secure the reagent pad cap 156 against the slider 146 to thereby provide secure coverage of the collector paper 144 to guard against contamination of the collector paper 144 during transportation or storage. In FIG. 42, it will be appreciated that in this embodiment, an RFID chip 160 can be inserted into the device to allow continuous and specific monitoring of the location of the device 140.

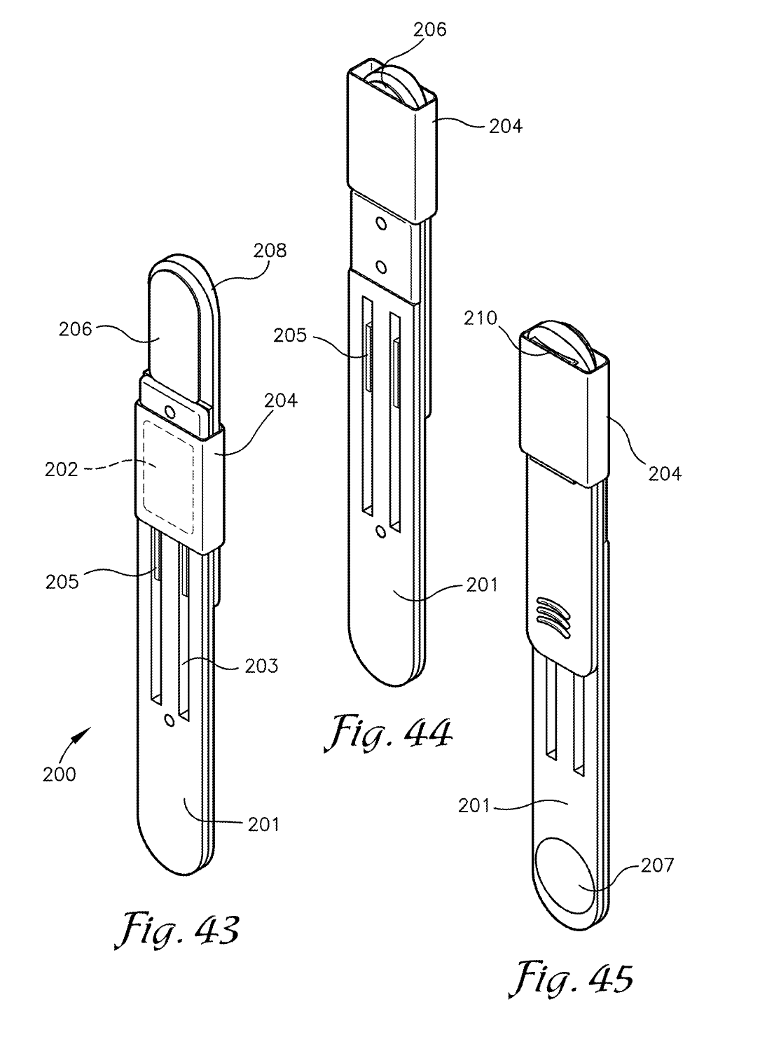

Referring now to FIGS. 43-45, embodiment 200 will be described wherein a reagent pad 202 is located on protective cap 204 which can be moved into a position over absorbent paper 206 extending from handle 201 once a specimen is collected thereon.

The device of embodiment 200 comprises a handle 201 comprising tracks 203 within which rails 205 of slider 208 can slide. Absorbent paper 206 extends from the handle 201. The protective cap 204 is slidably connected to, and partially encloses handle 201 and slider 208. In embodiment 200, the collector paper 206 is supported by slider 208 during specimen collection. This is shown in FIG. 43 where collector paper 206 extends across the slider 208 to receive support of the slider 208 when a specimen is being collected. Once a specimen has been collected, the protective cap 204, having the reagent pad 202 thereon, can be pressed upwardly as is shown in FIG. 44 to bring the reagent pad 202 (hidden in FIG. 44) into contact with the collector paper 206. The user can then press upon the cap 204 to transfer the reagent from the reagent pad 202 onto the paper 206. Alternatively, the protective cap 204 can be closely fitted to the dimensions of the support 208 having the paper 206 thereon such that as when the cap 204 is pressed upwardly, it compresses the paper 206 against the support 208 and against the pad 202 contained within the cap 204. FIG. 45 is a backside view of embodiment 200 showing this close fit configuration which is accomplished by the use of front ridge 210 on support 208, which serves to compress the slider 208 against the reagent pad 202 (hidden in FIG. 45) of cap 204. In FIG. 45, it will be appreciated that in this embodiment, an RFID chip 207 can be inserted into the device to allow continuous and specific monitoring of the location of the device 140.

### II. Stabilizing Composition

In another aspect, the present disclosure provides a stabilizing composition. Advantageously, a stabilizing composition of the present disclosure is capable of slowing down sample degradation and preserving biomolecule contents of collected specimens. A stabilizing composition of the disclosure may stabilize a collected specimen for a duration of days to weeks or even months or years. As will be appreciated by a skilled artisan, a stabilizing composition should not interfere with testing methods used in subsequent processing steps. For instance, a stabilizing composition should not interfere with nucleic acid testing methods, protein testing methods, or any other intended testing method.

A stabilizing composition may be used to stabilize a collected solid, fluid or particulate evidence sample related to any type of situation in which evidence collection is required. Such evidence collection can be associated with crime scenes or can simply be the collection of a biological sample from a human being at a crime scene, in the course of a traffic stop, or a paternity investigation. Suitable specimens for collection using the present devices are, in general, that evidence which is from a human being or located on a surface and which can be physically contacted by an evidence collection device to thereby obtain a sample of the evidence. Examples of such evidence specimens might be any type of biological fluid, either wet or dried, or any unknown substance which is visible or invisible and which can be located allowing for collection of a specimen of the evidence and capture of such a sample on a sample collector. Non-limiting examples of biological samples that may be collected and stabilized using a stabilizing dissolvable film formulation of the present disclosure include biological fluids and excretions isolated from any given subject or surface. In the context of the invention such samples include, but are not limited to, blood and fractions thereof, blood serum, blood plasma, urine, excreta, semen, seminal fluid, seminal plasma, prostatic fluid, pre-ejaculatory fluid (Cowper's fluid), pleural effusion, tears, saliva, sputum, sweat, biopsy, ascites, cerebrospinal fluid, amniotic fluid, lymph, marrow, cervical secretions, vaginal secretions, endometrial secretions, gastrointestinal secretions, bronchial secretions, breast secretions, ovarian cyst secretions, and tissue fluid samples.

A stabilizing composition may comprise at least one chelator, at least one surface acting agent, at least one antimicrobial agent, or combinations thereof. The various components of the stabilizing compositions and preferred stabilizing compositions are described below.

### A. Chelating Agent

Generally speaking, chelating agents deplete metal ions and are commonly used to deactivate metal-dependent enzymes to suppress damage to nucleic acids or proteins in a biological sample. A stabilizing composition of the present disclosure may comprise 1, 2, 3, 4, 5, 6, 7, 8, 9, or 10 or more chelating agents. Preferably, a stabilizing composition comprises 1, 2, 3, or 4 chelating agents. More preferably, a stabilizing composition comprises two chelating agents.

Chelating agents that may be used in a composition for stabilizing a biological sample are known in the art. In essence, any chelating agent capable of inhibiting the activity of metal-dependent enzymes may be used in a composition of the present disclosure. Non-limiting examples of suitable chelating agents include ethylenediamine tetracetic acid (EDTA) and its salts, N-(hydroxy-ethyl)ethylenediaminetriacetic acid, nitrilotriacetic acid (NIA), ethylene-bis(oxyethylene-nitrilo)tetraacetic acid (EGTA), 1,4,7,10-tetraazacyclodo-decane-N,N′,N″,N′″-tetraacetic acid (DOTA), 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane (DO3A), 1,4,7-triazacyclonane-N,N′,N″-triacetic acid (NOTA), 1,4,8,11-tetraazacyclotetra-decane-N,N′,N″,N′″-tetraacetic acid, diethylenetriamine-pentaacetic acid (DTPA), ethylenedicysteine, bis(aminoethanethiol)carboxylic acid, triethylenetetraamine-hexaacetic acid (TTNA), N-(hydroxyethyl)-ethylenediaminetriacetic acid (HEDTA), 1,2-diaminocyclohexane-N,N,N′,N′-tetraacetic acid, 1,2-bis(2-aminophenoxy)ethane-N,N,N′,N′-tetraacetic acid (BAPTA), 1-hydroxyethane 1,1-diphosphonic acid (HEDP), nitrilotriacetic acid (NTA), 2-hydroxyethyliminodiacetic acid disodium salt (HEIDA), 2-phosphono-1,2,4,-butanetricarboxylic acid (PBTC), carboxymethyl inulin, trisodium phosphate, sodium hexametaphosphate, sodium tripolyphosphate, tetrasodium pyrophosphate, potassium tripolyphosphate, tetrapotassium pyrophosphate, citric acid, gluconic acid, sodium gluconate, diethylenetriamine penta(methylene phosphonic acid) DTPMP, trans-1,2-cyclohexanediaminetetraacetic acid (CDTA), fusaric acid (FA), and picolinic acid (PA).

Preferably, chelating agents suitable for a composition of the present disclosure are divalent metal chelating agents. Non-limiting examples of divalent metal chelating agents include EDTA, EGTA, BAPTA, fusaric acid (FA), picolinic acid (PA), and trans-1,2-cyclohexanediaminetetraacetic acid (CDTA), or salts thereof. Preferably, a stabilizing composition of the present disclosure comprises EDTA and EGTA.

As will be appreciated by a skilled artisan, the amount of chelating agent added to a stabilizing composition can and will vary depending upon the identity of the collected biological sample. The concentration of a divalent metal chelator in a stabilizing composition of the disclosure is generally in the range of from about 0.1 mM to about 100 mM, preferably in the range of from about 1 mM to about 50 mM. More preferably, a stabilizing composition of the present disclosure comprises about 5 to about 15 mM EDTA and about 1 to about 5 mM EGTA.

### B. Surface Acting Agents

Stabilizing compositions of the present disclosure comprise at least one surface acting agent (alternatively referred to as a “surfactant” or “detergent”). Generally speaking, a surface acting agent may promote protein solubilization, membrane disruption, and cell permeabilization, thereby protecting a biological sample from degradation from microorganisms and enzymes.

A preservative composition of the present disclosure may comprise 1, 2, 3, 4, 5, 6, 7, 8, 9, or 10 or more surface acting agents. Preferably, a preservative composition comprises 1, 2, 3, or 4 surface acting agents. More preferably, a preservative composition comprises two surface acting agents.

As will be appreciated by a skilled artisan, any surface acting agent capable of stabilizing biological samples can be used in methods of the disclosure, provided that the agent does not interfere with testing methods used in subsequent processing steps. For instance, a surface acting agent may be an anionic surface acting agent, a cationic surface acting agent, a zwitterionic surface acting agent, a non-ionic surface acting agent, or combinations thereof.

Non-limiting examples of a cationic surface active agent include, but are not limited to, alkyltrimethylammonium bromide; benzalkonium chloride; benzalkonium chloride; benzyldimethylhexadecylammonium chloride; benzyldimethyltetradecylammonium chloride; benzyldodecyldimethylammonium bromide; benzyltrimethylammonium tetrachloroiodate; cetyltrimethylammonium bromide (CTAB); di methyldioctadecylammonium bromide; dodecylethyldi methylammonium bromide; dodecyltrimethylammonium bromide; dodecyltrimethylammonium bromide; dodecyltrimethylammonium chloride; ethylhexadecyldimethylammonium bromide; Girard's reagent T; hexadecyltrimethylammonium bromide; hexadecyltrimethylammonium bromide; N,N′,N′-polyoxyethylene(10)-N-tallow-1,3-diaminopropane; thonzonium bromide; and trimethyl(tetradecyl)ammonium bromide.

Non-limiting examples of a zwitterionic surface active agent include, but are not limited to, 3-[(3-cholamidopropyl)dimethylammonio]-2-hydroxy-1-propanesulfonate (CHAPSO); 3-[(3-cholamidopropyl)dimethylammonio]-1-propanesulfonate (CHAPS); 3-(4-Heptyl)phenyl-3-hydroxypropyl)dimethylammoniopropanesulfonate (C7BzO); 3-(N,N-dimethyloctylammonio) propanesulfonate inner salt (SB3-8); 3-(decyldimethylammonio) propanesulfonate inner salt (SB3-10; caprylyl sulfobetaine); 3-(dodecyldimethylammonio) propanesulfonate inner salt (SB3-12); 3-(N,N-dimethyltetradecylammonio)propanesulfonate (SB3-14); 3-(N,N-dimethylpalmitylammonio) propanesulfonate (SB3-16); 3-(N,N-dimethyloctadecylammonio) propanesulfonate (SB3-18); 3-[N,N-dimethyl(3-myristoylaminopropyl)ammonio]propanesulfonate (ASB-14). Other suitable zwitterionic detergents, depending on the embodiment, include: acetylated lecithin; apricotamidopropyl betaine; babassuamidopropyl betaine; behenyl betaine; bis 2-hydroxyethyl tallow glycinate; C12-14 alkyl dimethyl betaine; canolamidopropyl betaine; capric/caprylic amidopropyl betaine; capryloamidopropyl betaine; cetyl betaine; cocamidopropyl betaine; cocamidopropyl dimethylaminohydroxypropyl hydrolyzed collagen; N-[3-cocamido)-propyl]-N,N-dimethyl betaine, potassium salt; cocamidopropyl hydroxysultaine; cocamidopropyl sulfobetaine; cocaminobutyric acid; cocaminopropionic acid; cocoamphodipropionic acid; coco-betaine; cocodimethylammonium-3-sulfopropylbetaine; cocoiminodiglycinate; cocoiminodipropionate; coco/oleamidopropyl betaine; cocoyl sarcosinamide DEA; DEA-cocoamphodipropionate; dihydroxyethyl tallow glycinate; dimethicone propyl PG-betaine; N,N-dimethyl-N-lauric acid-amidopropyl-N-(3-sulfopropyl)-ammonium betaine; N,N-dimethyl-N-myristyl-N-(3-sulfopropyl)-ammonium betaine; N,N-dimethyl-N-palmityl-N-(3-sulfopropyl)-ammonium betaine; N,N-dimethyl-N-stearamidopropyl-N-(3-sulfopropyl)-ammonium betaine; N,N-dimethyl-N-stearyl-N-(3-sulfopropyl)-ammonium betaine; N,N-dimethyl-N-tallow-N-(3-sulfopropyl)-ammonium betaine; disodium caproamphodiacetate; disodium caproamphodipropionate; disodium capryloamphodiacetate; disodium capryloamphodi propionate; disodium cocoamphodiacetate; disodium cocoamphodipropionate; disodium isostearoamphodipropionate; disodium laureth-5 carboxyamphodiacetate; disodium lauriminodipropionate; disodium lauroamphodiacetate; disodium lauroamphodipropionate; disodium octyl b-iminodipropionate; disodium oleoamphodiacetate; disodium oleoamphodipropionate; disodium PPG-2-isodeceth-7 carboxyamphodiacetate; disodium soyamphodiacetate; disodium stearoamphodiacetate; disodium tallamphodipropionate; disodium tallowamphodiacetate; disodium tallowiminodipropionate; disodium wheatgermamphodiacetate; N,N-distearyl-N-methyl-N-(3-sulfopropyl)-ammonium betaine; erucamidopropyl hydroxysultaine; ethylhexyl dipropionate; ethyl hydroxymethyl oleyl oxazoline; ethyl PEG-15 cocamine sulfate; hydrogenated lecithin; hydrolyzed protein; isostearamidopropyl betaine; lauramidopropyl betaine; lauramidopropyl dimethyl betaine; lauraminopropionic acid; lauroamphodipropionic acid; lauroyl lysine; lauryl betaine; lauryl hydroxysultaine; lauryl sultaine; linoleamidopropyl betaine; lysolecithin; milk lipid amidopropyl betaine; myristamidopropyl betaine; octyl dipropionate; octyliminodipropionate; oleamidopropyl betaine; oleyl betaine; 4,4(5H)-oxazoledimethanol, 2-(heptadecenyl)-; palmitamidopropyl betaine; palmitamine oxide; ricinoleamidopropyl betaine; ricinoleamidopropyl betaine/IPDI copolymer; sesamidopropyl betaine; sodium C12-15 alkoxypropyl iminodipropionate; sodium caproamphoacetate; sodium capryloamphoacetate; sodium capryloamphohydroxypropyl sulfonate; sodium capryloamphopropionate; sodium carboxymethyl tallow polypropylamine; sodium cocaminopropionate; sodium cocoamphoacetate; sodium cocoamphohydroxypropyl sulfonate; sodium cocoamphopropionate; sodium dicarboxyethyl cocophosphoethyl imidazoline; sodium hydrogenated tallow dimethyl glycinate; sodium isostearoamphopropionate; sodium lauriminodipropionate; sodium lauroamphoacetate; sodium oleoamphohydroxypropylsulfonate; sodium oleoamphopropionate; sodium stearoamphoacetate; sodium tallamphopropionate; soyamidopropyl betaine; stearyl betaine; tallowamidopropyl hydroxysultaine; tallowamphopolycarboxypropionic acid; trisodium lauroampho PG-acetate phosphate chloride; undecylenamidopropyl betaine; and wheat germamidopropyl betaine.

Non-limiting examples of anionic surface active agents include, but are not limited to, amine dodecylbenzene sulfonate; ammonium capryleth sulfate; ammonium cumenesulfonate; ammonium dihydroxy stearate; ammonium dodecylbenzene sulfonate; ammonium laureth sulfate; ammonium laureth-12 sulfate; ammonium laureth-30 sulfate; ammonium lauryl sarcosinate; ammonium lauryl sulfate; ammonium lauryl sulfosuccinate; ammonium lignosulfonate; ammonium myreth sulfate; ammonium naphthalene sulfonate; ammonium nonoxynol-20 sulfate; ammonium nonoxynol-30 sulfate; ammonium nonoxynol-4 sulfate; ammonium nonoxynol-6 sulfate; ammonium nonoxynol-9 sulfate; ammonium oleic sulfate; ammonium perfluorooctanoate; ammonium stearate; ammonium xylenesulfonate; butyl naphthalene sulfonate; butyl phosphate; calcium dodecylbenzene sulfonate; calcium stearoyl lactylate; calcium tetrapropylenebenzene sulfonate; capryleth-9 carboxylic acid; cetyl phosphate; cumene sulfonic acid; DEA-cetyl phosphate; DEA-dodecylbenzene sulfonate; DEA-lauryl sulfate; deceth-4 phosphate; diammonium lauryl sulfosuccinate; diammonium stearyl sulfosuccinamate; diamyl sodium sulfosuccinate; dicyclohexyl sodium sulfosuccinate; dihexyl sodium sulfosuccinate; diisobutyl sodium sulfosuccinate; dilaureth-7 citrate; dimethiconol; dinonoxynol-4 phosphate; dioctyl ammonium sulfosuccinate; dioctyl sodium sulfosuccinate; disodium cetearyl sulfosuccinamate; disodium cocamido MEA-sulfosuccinate; disodium cocamido PEG-3 sulfosuccinate; disodium deceth-6 sulfosuccinate; disodium decyl diphenyl ether disulfonate; disodium dodecyloxy propyl sulfosuccinamate; disodium isodecyl sulfosuccinate; disodium laneth-5 sulfosuccinate; disodium lauramido DEA-sulfosuccinate; disodium lauramido MEA-sulfosuccinate; disodium laureth sulfosuccinate; disodium lauryl sulfosuccinate; disodium myristamido MEA-sulfosuccinate; disodium oleamido MEA-sulfosuccinate; disodium oleamido PEG-2 sulfosuccinate; disodium oleth-3 sulfosuccinate; disodium PEG-4 cocamido MIPA sulfosuccinate; disodium ricinoleamido MEA-sulfosuccinate; disodium stearyl sulfosuccinamate; disodium undecylenamido MEA-sulfosuccinate; ditridecyl sodium sulfosuccinate; dodecenylsuccinic anhydride; dodecyl diphenyl ether disulfonic acid; dodecyl diphenyloxide disulfonic acid; dodecylbenzenesulfonic acid; glyceryl dioleate SE; glyceryl distearate SE; glyceryl ricinoleate SE; glyceryl stearate citrate; glyceryl stearate SE; glycol stearate SE; hexyl phosphate; isopropyl phosphate; isopropylamine dodecylbenzenesulfonate; isosteareth-2 phosphate; isotrideceth-3 phosphate; isotrideceth-6 phosphate; laureth-1 phosphate; laureth-12 carboxylic acid; laureth-3 phosphate; laureth-4 phosphate; laureth-6 phosphate; laureth-7 citrate; laureth-9 phosphate; lauryl phosphate; lithium lauryl sulfate; magnesium laureth sulfate; magnesium PEG-3 cocamide sulfate; MEA-laureth phosphate; MEA-lauryl sulfate; MIPA-laureth sulfate; MIPA-lauryl sulfate; myristoyl sarcosine; naphthalene-formaldehyde sulfonate; nonoxynol-10 phosphate; nonoxynol-12 phosphate; nonoxynol-3 phosphate; nonoxynol-4 phosphate; nonoxynol-4 sulfate; nonoxynol-6 phosphate; nonoxynol-7 phosphate; nonoxynol-8 phosphate; nonoxynol-9 phosphate; nonyl nonoxynol-10 phosphate; nonyl nonoxynol-15 phosphate; nonyl nonoxynol-7 phosphate; oleth-10 carboxylic acid; oleth-10 phosphate; oleth-3 carboxylic acid; oleth-4 phosphate; oleth-5 phosphate; oleth-6 carboxylic acid; oleth-7 phosphate; PEG-2 dilaurate SE; PEG-2 dioleate SE; PEG-2 distearate SE; PEG-2 laurate SE; PEG-2 oleate SE; PEG-2 stearate SE; PEG-9 stearamide carboxylic acid; potassium cetyl phosphate; potassium deceth-4 phosphate; potassium dodecylbenzene sulfonate; potassium isosteareth-2 phosphate; potassium lauroyl sarcosinate; potassium lauryl sulfate; potassium oleate; potassium oleic sulfate; potassium perfluorooctoate; potassium ricinoleic sulfate; PPG-2 laurate SE; PPG-2 oleate SE; PPG-2 stearate SE; PPG-5-ceteth-10 phosphate; propylene glycol laurate SE; propylene glycol oleate SE; propylene glycol ricinoleate SE; propylene glycol stearate SE; PVM/MA copolymer; sodium 2-ethylhexyl phosphate; sodium 2-ethylhexyl sulfate; sodium a olefin sulfonate; sodium allyloxy hydroxypropyl sulfonate; sodium behenoyl lactylate; sodium butoxyethoxy acetate; sodium butyl naphthalene sulfonate; sodium butyl oleate sulfate; sodium butyl oleate sulfonate; sodium butyl phosphate; sodium caproyl lactylate; sodium caprylyl sulfonate; sodium cetyl sulfate; sodium cholate; sodium cumenesulfonate; sodium deceth sulfate; sodium decyl diphenyl ether sulfonate; sodium decyl sulfate; sodium deoxycholate; sodium dibutyl naphthalene sulfonate; sodium didodecylbenzene sulfonate; sodium diisooctyl sulfosuccinate; sodium diisopropyl naphthalene sulfonate; sodium dilaureth-7 citrate; sodium dinonyl sulfosuccinate; sodium dodecyl diphenyl ether disulfonate; sodium dodecyl diphenyloxide disulfonate; sodium dodecylbenzenesulfonate; sodium glyceryl trioleate sulfate; sodium hexadecyl diphenyl disulfonate; sodium hexadecyl diphenyloxide disulfonate; sodium hexyl diphenyloxide disulfonate; sodium isothionate; sodium isodecyl sulfate; sodium isooctyl sulfate; sodium isostearoyl lactylate; sodium isotrideceth-15 sulfate; sodium lactate; sodium lauramido DEA-sulfosuccinate; sodium laureth phosphate; sodium laureth sulfate (sodium dodecyl sulfate or SDS); sodium laureth sulfosuccinate; sodium laureth-10 phosphate; sodium laureth-11 carboxylate; sodium laureth-12 sulfate; sodium laureth-13 acetate; sodium laureth-13 carboxylate; sodium laureth-3 carboxylate; sodium laureth-4 carboxylate; sodium laureth-4 phosphate; sodium laureth-6 carboxylate; sodium laureth-7 carboxylate; sodium laureth-7 sulfate; sodium laureth-8 sulfate; sodium lauroyl glutamate; sodium lauroyl lactylate; sodium lauroyl lactylate; sodium lauroyl methylaminopropionate; sodium lauroyl sarcosinate; sodium lauryl phosphate; sodium lauryl sulfate; sodium lauryl sulfoacetate; sodium lignate; sodium lignosulfonate; sodium methallyl sulfonate; sodium methyl lauroyl taurate; sodium methyl myristoyl taurate; sodium methyl oleoyl taurate; sodium methyl palmitoyl taurate; sodium methyl stearoyl taurate; sodium methylnaphthalenesulfonate; sodium m-nitrobenzenesulfonate; sodium myreth sulfate; sodium myristoyl glutamate; sodium myristoyl sarcosinate; sodium myristyl sulfate; sodium nonoxynol sulfate; sodium nonoxynol-10 sulfate; sodium nonoxynol-10 sulfosuccinate; sodium nonoxynol-15 sulfate; sodium nonoxynol-4 sulfate; sodium nonoxynol-5 sulfate; sodium nonoxynol-6 phosphate; sodium nonoxynol-6 sulfate; sodium nonoxynol-8 sulfate; sodium nonoxynol-9 phosphate; sodium nonoxynol-9 sulfate; sodium octoxynol-2 ethane sulfonate; sodium octoxynol-3 sulfate; sodium octyl sulfate; sodium octylphenoxyethoxyethyl sulfonate; sodium oleic sulfate; sodium oleth-7 phosphate; sodium oleyl phosphate; sodium oleyl sulfate; sodium oleyl sulfosuccinamate; sodium palmitoyl sarcosinate; sodium phenyl sulfonate; sodium propyl oleate sulfate; sodium stearoyl lactylate; sodium stearyl sulfosuccinamate; sodium trideceth sulfate; sodium trideceth-3 carboxylate; sodium trideceth-6 carboxylate; sodium trideceth-7 carboxylate; sodium tridecyl sulfate; sodium tridecylbenzene sulfonate; sodium xylenesulfonate; stearoyl sarcosine; TEA-lauroyl glutamate; TEA-lauryl sulfate; tetrasodium dicarboxyethyl stearyl sulfosuccinamate; TIPA-laureth sulfate; triceteareth-4 phosphate; triceteth-5 phosphate; trideceth-2 phosphate; trideceth-3 phosphate; trideceth-5 phosphate; tridecyl phosphate; and trilaureth-4 phosphate; and trioctyl phosphate.

Non-limiting examples of non-ionic surface active agents include, but are not limited to, polyoxyethylene (10) cetyl ether (BRIJ® 56); polyoxyethylene (20) cetyl ether (BRIJ® 58); polyoxyethyleneglycol dodecyl ether (BRIJ® 35); polyoxyethylene (9) p-t-octyl phenol (NONIDET™ P-40); polyoxyethylene (4-5) p-t-octyl phenol (TRITON™ X-45); polyoxyethylene (7-8) p-t-octyl phenol (TRITON™ X-114); polyoxyethylene (9-10) p-t-octyl phenol (TRITON™ X-100); polyoxyethylene (9-10) nonylphenol (TRITON™ N-101); a polysorbate surface active agent such as polyoxyethylene (20) sorbitol monolaurate (TWEEN® 20), polyoxyethylene (20) sorbitol monopalmitate (TWEEN® 40), Polyoxyethylene (20) sorbitan monostearate (Tween® 60), and polyoxyethylene (20) sorbitol monooleate (TWEEN® 80); dimethyldecylphosphine oxide (APO-10); dimethyldodecylphosphine oxide (APO-12); cyclohexyl-n-ethyl-β-D-maltoside; cyclohexyl-n-hexyl-β-D-maltoside; cyclohexyl-n-methyl-β-maltoside; n-decanoylsucrose; n-decyl-β-D-glucopyranoside; n-decyl-β-maltopyranoside; n-decyl-β-D-thiomaltoside; n-dodecanoyl sucrose; decaethylene glycol monododecyl ether; N-decanoyl-N-methylglucamine; n-decyl α-D-glucopyranoside; decyl β-D-maltopyranoside; n-dodecanoyl-N-methylglucamide; n-dodecyl α-D-maltoside; n-dodecyl β-D-maltoside; heptane-1,2,3-triol; heptaethylene glycol monodecyl ether; heptaethylene glycol monododecyl ether; heptaethylene glycol monotetradecyl ether; n-hexadecyl β-D-maltoside; hexaethylene glycol monododecyl ether; hexaethylene glycol monohexadecyl ether; hexaethylene glycol monooctadecyl ether; hexaethylene glycol monotetradecyl ether; methyl-6-O—(N-heptylcarbamoyl)-α-D-glucopyranoside; nonaethylene glycol monododecyl ether; N-nonanoyl-N-methylglucamine; N-nonanoyl-N-methylglucamine; octaethylene glycol monodecyl ether; octaethylene glycol monododecyl ether; octaethylene glycol monohexadecyl ether; octaethylene glycol monooctadecyl ether; octaethylene glycol monotetradecyl ether; octyl-β-glucoside; octyl-β-thioglucoside; octyl-β-D-glucopyranoside; octyl-β-D-1-thioglucopyranoside; pentaethylene glycol monodecyl ether; pentaethylene glycol monododecyl ether; pentaethylene glycol monohexadecyl ether; pentaethylene glycol monohexyl ether; pentaethylene glycol monooctadecyl ether; pentaethylene glycol monooctyl ether; polyethylene glycol diglycidyl ether; polyethylene glycol ether; polyoxyethylene 10 tridecyl ether; polyoxyethylene (100) stearate; polyoxyethylene (20) isohexadecyl ether; polyoxyethylene (20) oleyl ether; polyoxyethylene (40) stearate; polyoxyethylene (50) stearate; polyoxyethylene (8) stearate; polyoxyethylene bis(imidazolyl carbonyl); polyoxyethylene (25) propylene glycol stearate; saponin from Quillaja bark; tetradecyl-β-D-maltoside; tetraethylene glycol monodecyl ether; tetraethylene glycol monododecyl ether; tetraethylene glycol monotetradecyl ether; triethylene glycol monodecyl ether; triethylene glycol monododecyl ether; triethylene glycol monohexadecyl ether; triethylene glycol monooctyl ether; triethylene glycol monotetradecyl ether; tyloxapol; n-undecyl β-D-glucopyranoside, (octylphenoxy)polyethoxyethanol (IGEPAL® CA-630); polyoxyethylene (5) nonylphenylether (IGEPAL® CO-520); and polyoxyethylene (150) dinonylphenyl ether (IGEPAL® DM-970). In one embodiment, a surface active agent is polyoxyethylene (5) nonylphenylether (IGEPAL® CO-520). In another embodiment, a surface active agent is polyoxyethylene (150) dinonylphenyl ether (IGEPAL® DM-970). In one embodiment, a surface active agent is preferably (octylphenoxy) polyethoxyethanol (IGEPAL® CA-630).

Preferably, a suitable surface acting agent is an anionic surface acting agent. A preferred anionic surface acting agent may be SDS. Also preferably, a suitable surface acting agent is a non-ionic surface acting agent. A preferred non-ionic surface active agent may be a polysorbate surface acting agent such as Tween® 20, Tween® 40, Tween® 60, or Tween® 80, preferably Tween® 20. More preferably, a stabilizing composition of the present disclosure comprises an anionic surfactant and a non-ionic surfactant. Most preferably, a stabilizing composition of the present disclosure comprises SDS and Tween® 20.

As will be appreciated by a skilled artisan, the amount of surface acting agent added to the biological fluid can and will vary depending upon the identity of the collected biological sample. When a stabilizing composition of the present disclosure comprises a polysorbate surface acting agent, the weight fraction of the polysorbate surface acting agent in compositions of the present disclosure may be in the range of from about 0.001% to about 0.1%, preferably in the range of from about 0.005% to about 0.015%. When a stabilizing composition of the present disclosure comprises SDS, the concentration of SDS in compositions of the present disclosure may be in the range of from about 1 mM to about 10 mM, preferably in the range of from about 4 mM to about 8 mM. More preferably, a preservative composition of the present disclosure comprises about 0.005% to about 0.015% Tween® 20 and about 4 mM to about 8 mM SDS.

### C. Antimicrobial

Stabilizing compositions of the present disclosure comprise at least one antimicrobial to inhibit or prevent inadvertent microbial growth in a collected biological sample. A stabilizing composition of the present disclosure may comprise 1, 2, 3, 4, 5, 6, 7, 8, 9, or 10 or more antimicrobials. Preferably, a stabilizing composition comprises 1, 2, 3, or 4 antimicrobials. More preferably, a stabilizing composition comprises one antimicrobial.

Non-limiting examples of antimicrobials that may be suitable for use in a stabilizing composition of the present disclosure include antibiotics such as the sulfate salts of gentamicin, chromamphenicol, and streptomycin, parabens such as methyl paraben and propyl paraben, chlorobutanol, phenolic compounds, protease inhibitors, glutaraldehyde, benzoic acid, quaternary ammonium salts, chlorhexidine digluconate, bronopol, hydrogen peroxide, sodium dichloroisocyanurate, sodium hypochlorite, Proclin® 300, Proclin® 150 (5-chloro-2-methyl-4-isothiazolin-3-one, and 2-methyl-4-isothiazolin-3-one, mercury-containing salts, sulfate salts of gentamicin, chloramphenicol and streptomycin, sodium benzoate, potassium sorbate, sodium azide, and combinations thereof. A preferred antimicrobial is sodium azide. The weight fraction of sodium azide in a stabilizing composition of the disclosure may be in the range of from about 0.01% to about 0.1%. More preferably, a stabilizing composition of the present disclosure comprises about 0.04% to about 0.06% sodium azide.

### D. Other Components

In order to maintain a pH appropriate for preserving and stabilizing a collected biological sample, a buffer is typically incorporated into a stabilizing composition of the present disclosure. A variety of buffers are suitable for use in stabilizing compositions of the present disclosure. By way of non-limiting example, the buffers may include, but are not limited to, 3-{[tris(hydroxymethyl)methyl]amino}propanesulfonic acid (TAPS), N,N-bis(2-hydroxyethyl)glycine (Bicine), tris(hydroxymethyl)methylamine (tris), N-tris(hydroxymethyl)methylglycine (tricine), 3-[N-Tris(hydroxymethyl)methylamino]-2-hydroxypropanesulfonic Acid (TAPSO), 4-2-hydroxyethyl-1-piperazineethanesulfonic acid (HEPES), 2-{[tris(hydroxymethyl)methyl]amino}ethanesulfonic acid (TES), 3-(N-morpholino)propanesulfonic acid (MOPS), piperazine-N,N′-bis(2-ethanesulfonic acid) (PIPES), dimethylarsinic acid (cacodylate), saline sodium citrate (SSC), 2-(N-morpholino)ethanesulfonic acid (MES), and 2(R)-2-(methylamino)succinic acid (succinic acid). A buffer may be incorporated into a stabilizing composition alone or as a combination of two or more buffers. Preferably, the buffer is tris.

The concentration of a buffer in a composition is typically sufficient to maintain a desired pH range. For instance, the concentration of buffer in presently described stabilizing compositions may range from about 20 mM to about 100 mM. Preferably, the concentration of a buffer in stabilizing compositions ranges from about 40 mM to about 60 mM. More preferably, the concentration of buffer in stabilizing compositions is about 50 mM.

The pH of preservative compositions may be adjusted to a pH of more than about 6.0 and more preferably, more than about 7.0. Preferably, a stabilizing composition has a pH that ranges from about 7.0 to about 10.0. More preferably, the pH of a stabilizing composition is adjusted to range from about 8.0 to about 9.5. For instance, the pH of a stabilizing composition may be adjusted to a pH of about 8.0, 8.1, 8.2, 8.3, 8.4, 8.5, 8.6, 8.7, 8.8, 8.9, 9.0, 9.1, 9.2, 9.3, 9.4, or a pH of about 9.5. Preferably, the pH of a stabilizing composition is adjusted to a pH of about 8.2, 8.21, 8.22, 8.23, 8.24, 8.25, 8.26, 8.27, 8.28, 8.29, 8.3, 8.31, 8.32, 8.33, 8.34, 8.35, 8.36, 8.37, 8.38, 8.39, 8.4, 8.41, 8.42, 8.43, 8.44, 8.45, 8.46, 8.47, 8.48, 8.49, or a pH of about 8.5. Preferably, the pH of a stabilizing composition is adjusted to a pH of about 8.31. Preferably, the pH of compositions is adjusted using hydrochloric acid.

Preferably, the buffer comprises tris at a concentration of about 50 mM, and the pH of the tris buffer is adjusted to about 8.31 using hydrochloric acid to produce the tris-HCl salt. Alternatively, concentrated tris-HCl buffer solution may be prepared at the desired pH, and a preservative composition may be prepared using the concentrated buffer solution.

Stabilizing compositions may further comprise salts at concentrations suitable for testing methods used in subsequent processing steps. For instance, a stabilizing composition may comprise sodium chloride (NaCl) or potassium chloride (KCl). Preferably, a stabilizing composition comprises KCl at a concentration ranging from about 5 mM to about 100 mM or more. More preferably, a stabilizing composition comprises KCl salt at a concentration ranging from about 20 mM to about 30 mM.

Stabilizing compositions may also further comprise a dye to impart a color or a fluorescence to a composition and to a collection absorbent contacted with a composition of the present disclosure. Color or fluorescence may provide visual evidence or a detectable light absorption or light emission evidencing that a stabilizing composition has been dissolved, dispersed, and transferred to a collection absorbent contacted with a dissolvable stabilizing composition. Non-limiting examples of fluorescent dyes include fluorescein, cyanine, Texas Red, ROX, FAM, JOE, SYBR Green, OliGreen, HEX. In addition to these fluorescent dyes, ultraviolet/visible dyes, such as dichlorophenol, indophenol, saffranin, crystal violet, and commercially-available food coloring can also be used. Suitable coloring dyes may include, but are not limited to, ultraviolet/visible dyes, such as dichlorophenol, indophenol, saffranin, crystal violet food, drug and cosmetic colors (FD&C), drug and cosmetic colors (D&C), external drug and cosmetic colors (Ext. D&C), and other dyes known in the industry. Preferably, a stabilizing composition of the present disclosure comprises blue food coloring. As will be appreciated by a skilled artisan, the concentration of a dye in a stabilizing composition may be any concentration sufficient to provide evidence that a stabilizing composition has been transferred to a collection absorbent without interfering with testing methods used in subsequent processing steps.

Stabilizing compositions may also further comprise other agents, including reducing agents such as dithiothreitol (DTT), β-mercaptoethanol (BME), and tris(2-carboxyethyl)phosphine (TCEP), bulking agents such as dextran sulfate, polyethylene glycol (PEG), and tetraethylene glycol, plasticizers such as glycerol, di-butylpthallate, and polyethylene glycols, and others.

In general, presently described stabilizing compositions are prepared by dissolving components of the compositions described herein in a solvent to generate a stabilizing solution. Any solvent capable of dissolving components of the presently described stabilizing compositions may be used, provided the solvent can also dissolve film-forming agents of the present disclosure, and provided the solvent is compatible with film-forming methods described further below. As such, a solvent may be an aprotic solvent, a protic solvent, an organic solvent, or combinations thereof.

Non-limiting examples of suitable aprotic solvents include acetone, acetonitrile, diethoxymethane, N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, N,N-dimethylacetamide (DMAC), 1,4-dioxane, N-methyl-2-pyrrolidinone (NMP), ethyl acetate, ethyl formate, ethyl methyl ketone, formamide, hexachloroacetone, hexamethylphosphoramide, methyl acetate, N-methylacetamide, N-methylformamide, methylene chloride, nitrobenzene, nitromethane, propionitrile, sulfolane, tetramethylurea, tetrahydrofuran (THF), 2-methyl tetrahydrofuran, and trichloromethane.

Suitable examples of protic solvents include, but are not limited to, methanol, ethanol, isopropanol, n-propanol, isobutanol, n-butanol, s-butanol, t-butanol, isopropyl alcohol (IPA), formic acid, acetic acid, and water.

Suitable organic solvents include, but are not limited to, alkane and substituted alkane solvents (including cycloalkanes), aromatic hydrocarbons, esters, ethers, ketones, combinations thereof, and the like. Specific organic solvents that may be employed include, for example, acetonitrile, benzene, butyl acetate, t-butyl methylether, t-butyl methylketone, chlorobenzene, chloroform, chloromethane, cyclohexane, dichloromethane, dichloroethane, diethyl ether, ethyl acetate, diethylene glycol, fluorobenzene, heptane, hexane, isobutylmethylketone, isopropyl acetate, methyl ethyl ketone, methyltetrahydrofuran, pentyl acetate, n propyl acetate, tetrahydrofuran, and toluene.

Preferably, components of stabilizing compositions of the present disclosure are dissolved in one or more protic solvents to form a stabilizing solution. Preferred protic solvents include water and IPA. For instance, components may be dissolved in water and about 5% to about 40% IPA, more preferably in water and about 20% IPA. More preferably, components of stabilizing compositions of the present disclosure are dissolved in water to form a stabilizing solution.

### E. Preferred Stabilizing Compositions

In one preferred embodiment, a stabilizing composition of the present disclosure comprises at least one chelator and at least one surface acting agent. In a preferred alternative of the embodiments, the stabilizing composition comprises EDTA and Tween®-20. In an exemplary alternative of the embodiments, the stabilizing composition comprises 50 mM EDTA and 0.01% Tween®-20.

In another preferred embodiment, a stabilizing composition of the present disclosure comprises at least one chelator, at least one surface acting agent, and at least one antimicrobial agent. In a preferred alternative of the embodiments, the stabilizing composition comprises EDTA, Tween®-20, and sodium azide. In an exemplary alternative of the embodiments, the stabilizing composition comprises 50 mM EDTA, 0.1% Sodium Azide, and 0.01% Tween®-20. In a particularly exemplary alternative of the embodiments, the stabilizing composition comprises about 5 to about 15 mM EDTA, about 1 to about 5 mM EGTA, about 0.001% to about 0.1% Tween, about 1 to about 10% SDS, about 0.01 to about 0.1 sodium azide, about 20 to about 30 mM KCl, and about 40 to about 60 mM Tris-HCl.

### III. Stabilizing Dissolvable Film

In yet another aspect, the present disclosure provides formulations of a stabilizing composition in the form of a dissolvable film. The stabilizing dissolvable film is capable of stabilizing a collected biological sample. In such aspects, the stabilizing dissolvable film is included in specimen collection devices of the present disclosure.

In accordance with the present disclosure, stabilizing dissolvable film formulations comprising stabilizing compositions are capable of dissolving upon contacting a collected sample on a device of the disclosure and transferring the stabilizing composition to the surface of an absorbent sample collector. A stabilizing dissolvable film formulation dissolves upon contact with a wet sample collection absorbent and percolates through the sample collected on the collection absorbent, thereby delivering a stabilizing composition to the collected sample. Advantageously, a film formulation of the present disclosure provides for efficient penetration into a collected sample, and allows for hands-free application of a stabilizing composition without interfering with subsequent sample testing steps.

A stabilizing dissolvable film of the present disclosure comprises a stabilizing composition and a film-forming agent. Stabilizing compositions may be as described in Section II. Film-forming agents and methods of preparing stabilizing dissolvable films are described below.

As described further below, a stabilizing dissolvable film is prepared by preparing a film-forming stabilizing composition in a solvent, and drying or removing the solvent to generate a dried film comprising only non-volatile components. Weight fractions and concentrations of components in stabilizing film-forming compositions described herein throughout refer to amounts and concentrations of components in stabilizing film-forming compositions before the solvent is removed to form a stabilizing dissolvable film.

### A. Film-Forming Agent

A stabilizing dissolvable film formulation comprises a film-forming agent. A film-forming agent is preferably a water soluble polymer. As used herein, the term “water soluble” means that the water soluble agent can be dissolved, dispersed, or suspended in water. A dissolvable preservative film formulation of the present disclosure may comprise 1, 2, 3, 4, 5, 6, 7, 8, 9, or 10 or more film-forming agents. Preferably, a preservative composition comprises 1, 2, 3, or 4 film-forming agents. More preferably, a preservative composition comprises one film-forming agent.

Water soluble agents capable of forming dissolvable film are known in the art and may be synthetic, natural, or modified. Non-limiting examples of film-forming agents include wheat or soybean proteins, keratin, for example keratin hydrolysates and sulfonic keratins, casein, albumin, collagen derivatives, glutelin, glucagon, gluten, zein, gelatins and derivatives thereof, polymers derived from chitin or from chitosan, such as anionic, cationic, amphoteric or nonionic chitin or chitosan polymers, polysaccharide polymers such as cellulose-based polymers, for instance hydroxyethylcellulose, hydroxypropylcellulose, hydroxypropylmethylcellulose, methylcellulose, ethylhydroxyethylcellulose, carboxymethylcellulose and quaternized cellulose derivatives, starches and derivatives thereof, acrylic polymers or copolymers such as polyacrylates, polymethacrylates and copolymers thereof, vinyl polymers such as polyvinylpyrrolidones, copolymers of methyl vinyl ether and of maleic anhydride, the copolymer of vinyl acetate and of crotonic acid polymers, adipic acid polymers, copolymers of vinylpyrrolidone and of vinyl acetate, copolymers of vinylpyrrolidone and of caprolactam, polyvinyl alcohols, polymers of natural origin, which are optionally modified, such as gum arabic, carob bean gum, guar gum, xanthan derivatives, cellulose gum, or karaya gum, alginates, carrageenans, ulvanes and other algal colloids, glycoaminoglycans, hyaluronic acid and its derivatives, shellac, sandarac gum, dammar resins, elemi gums and copal resins, deoxyribonucleic acid, mucopolysaccharides such as hyaluronic acid, chondroitin sulphate, caprolactams, pullulan, pectin, mannan and galactomannans, and glucomannans, carageenans, and mixtures and/or derivatives thereof.

A preferred film-forming agent is polyvinylpyrrolidone (PVP). PVP, also commonly known as polyvidone or povidone, is a water-soluble polymer made from the monomer N-vinylpyrrolidone and may have molecular weights ranging from about 1000 Daltons (1K) to about 1 million K. Preferably, a PVP having a molecular weight about 20K to about 100K is used to prepare a dissolvable film of the present disclosure. More preferably, PVP 40K is used to prepare a dissolvable film.

Another preferred film-forming agent is carboxymethyl cellulose (CMC). CMC or cellulose gum is a cellulose derivative with carboxymethyl groups (—CH₂—COOH) bound to some of the hydroxyl groups of the glucopyranose monomers that make up the cellulose backbone. The functional properties of CMC may depend on the degree of substitution of the cellulose structure (i.e., how many of the hydroxyl groups have taken part in the substitution reaction), as well as the chain length of the cellulose backbone structure and the degree of clustering of the carboxymethyl substituents.

Using a film-forming agent, a stabilizing film-forming solution is prepared. A stabilizing film-forming composition may be prepared by providing a stabilizing solution as described in Section II above, and dissolving a film-forming agent in the stabilizing solution. As will be appreciated by a skilled artisan, the amount of film-forming agent can and will vary depending on the film-forming agent and the intended physical properties of a dissolvable film such as rigidity and dissolution time. For instance, a water soluble film-forming agent is present in an amount of from about 0.1% to about 20% by weight or more of the film-forming solution.

When a preferred film-forming agent is PVP, a stabilizing film-forming solution may be prepared by dissolving PVP in an amount of from about 1% to about 50% by weight of a film-forming solution. More preferably, PVP is present in an amount of from about 1% to about 10% by weight, and even more preferably in an amount of about 5% by weight.

When a preferred film-forming agent is CMC, a stabilizing film-forming solution may be prepared by dissolving CMC in an amount of from about 0.1% to about 5% by weight of a film-forming solution. More preferably, CMC is present in an amount from about 0.1% to about 1% by weight, and even more preferably in an amount of about 0.25% to about 0.5% by weight.

### B. Stabilizing Dissolvable Film

A stabilizing dissolvable film is generally prepared by preparing a film-forming solution as described in Section IIIA that is cast, extruded, or otherwise processed, to become a stabilizing dissolvable film. Methods of preparing films from film-forming solutions are well known in the art. For instance, dissolvable film may be prepared using the methods of solvent casting, semi-solid casting, hot melt extrusion, solid dispersion extrusion, and forming on a liner.

In semi-solid casting, a solution of water soluble film-forming polymer is mixed with a solution of acid insoluble polymer to form a homogenous viscous solution. After sonication, the viscous solution is coated on non-treated casting film and dried. In hot melt extrusion, components of a film-forming composition are first mixed in solid form, and added to an extruder. The extruder melts the mixture and shapes it into a film.

Preferably, dissolvable preservative films of the present disclosure are prepared using a solvent casting method. The solvent casting method comprises casting a prepared dissolvable film solution on a flat surface and drying the film solution to generate a dissolvable film that could be manipulated. Non-limiting examples of drying methods may include freeze drying, vacuum drying, and air (or inert gas) drying at room temperature, or an elevated temperature, such as forced air drying, may be used to remove the solvent or fluid after coating.

Also preferably, dissolvable preservative films of the present disclosure are prepared by forming on a liner thereby generating a dissolvable film supported by the liner. In this method, a prepared dissolvable film solution may be applied to a liner and dried using any of the methods of drying as described above. A liner may be any flat surface onto which a dissolvable film solution may be applied to provide support to a dissolvable film. A liner may be rigid. Alternatively, a liner may be flexible. Additionally, a liner may be permeable, impermeable, or may comprise one or more layers, wherein some layers may be permeable and others may be impermeable. For instance, a liner may comprise at least two layers, wherein one of the layers is impermeable, and another layer is absorbent. When a liner comprises a permeable layer, dissolvable film solution may percolate into the permeable layer before drying to form a dissolvable preservative film embedded in the permeable liner. Alternatively, when a liner comprises an impermeable layer, a dissolvable preservative film may be formed on a surface of an impermeable liner. Alternatively, when a liner comprises at least two layers, wherein one of the layers is impermeable and another layer is permeable, a dissolvable preservative film may be formed on the permeable layer wherein a dissolvable film solution percolates into the permeable layer before drying to form a dissolvable preservative film embedded in the permeable layer and having an impermeable backing.

Any material capable of supporting a dissolvable preservative film of the present disclosure and is sufficiently strong for a method of use of the preservative film may be used as a liner, provided the liner does not interfere with functions of the film. Non-limiting examples of a liner material which may be appropriate for the present invention include glass fiber, nylon or polyester, and cellulose fiber such as paper or cotton. Non-limiting examples of a liner which may be appropriate for the present invention include S&S® 903™ paper, S&S® IsoCode® paper, and S&S® 900™ paper manufactured by Schleicher & Schuell, Inc., Whatman FTA paper from Whatman, Inc., RAETON™ 16, RAETON™ 26, RAETON™ 96, and RAETON™ 7 paper obtained from Griff Applied Laminates, and RG paper, LL72 paper, and B-85 paper from I.W. Tremont.

Methods of forming dissolvable film supported by a liner may include pouring a preservative film onto a liner, dipping a liner in a preservative film solution, or using rolling methods of forming dissolvable film on a liner. Using a rolling method, a dissolvable film-forming solution comprising a stabilizing composition of the present disclosure is rolled onto a backing substrate (liner) and dried to generate a dissolvable film on the backing liner.

A stabilizing dissolvable film of the present disclosure may be prepared at various thicknesses. As will be appreciated by a skilled artisan, the thickness of a stabilizing dissolvable film may determine the amount and/or concentration of stabilizing composition that may be transferred to a collected biological sample; a thicker stabilizing dissolvable film may deliver a larger amount of stabilizer to a collected biological sample, whereas a thinner stabilizing dissolvable film may deliver a smaller amount of stabilize to a sampler. As such, the thickness of a stabilizing dissolvable film may be tuned to deliver an ideal amount of a stabilizing composition to a biological sample. In general the thickness of a stabilizing dissolvable film is tuned to deliver an amount of stabilizing composition sufficient to stabilize a collected sample under describable storage and moving conditions, and for a desired period of time. The thickness of a stabilizing dissolvable film may be determined experimentally using methods known in the art. In general, the thickness of a stabilizing film composition may range from about 1μ to about 5 mm or more. Additionally, when a dissolvable film of the present disclosure is embedded in a permeable liner, the thickness of a stabilizing film composition may correspond to the thickness of the permeable liner. Additionally, when a dissolvable film of the present disclosure is embedded in a permeable liner, the thickness of a stabilizing film composition may extend beyond the thickness of the liner.

A stabilizing dissolvable film may be included in a cassette or reagent pad in a specimen collector with reagent lined cassette. For instance, a stabilizing dissolvable film may be included in a cassette or reagent pad in a specimen collector with reagent lined cassette wherein the cassette or reagent pad is as described in Section I above. As such, a stabilizing dissolvable film may be dimensioned to fit within the allotted space of the device for processing sample material. A stabilizing dissolvable film dimensioned to fit within the allotted space of a device may be dimensioned to cover part of a surface of a sample collector. Alternatively, a stabilizing dissolvable film dimensioned to fit within the allotted space of a device may be dimensioned to cover all of a surface of a sample collector.

A stabilizing dissolvable film of the present disclosure may also be dimensioned to provide a pre-determined amount of a stabilizing reagent. Additionally, the shape of a dissolvable film may be any shape that can be cut from a film or alternatively spot coated on a support or any shape, including a triangle, a square, a rectangle, a trapezoid, a circle, an oval, and a combination thereof.

Dimensioning a dissolvable film may be accomplished by a variety of methods known in the art. For example, a rule die, rotary die cutter, or a punch can be made and used for cutting the coated support film into pieces sized to fit within a sample collection device.

A preservative dissolvable film of the present disclosure is sufficiently rigid to be dimensionally stable during preparation, dimensioning, placement in a device, and downstream use in a device. By “dimensionally stable” is meant that the support film maintains its shape and dimensions to within about 5%, preferably within about 1%, of the shape and dimensions of the preservative film after preparation.

A stabilizing dissolvable film dissolves upon contact a wet sample collection absorbent to release a sample-stabilizing composition to a sample collected on a collection absorbent. As used herein, the term “dissolution time” refers to the duration of time required for a film of the present disclosure to dissolve and release a sample-stabilizing amount of stabilizing composition to a sample collected on a collection absorbent. As it will be apparent to those skilled in the art, dissolution time can and will vary depending on the composition and thickness of a stabilizing film, and on the level of wetness of a sample on a sample collection absorbent. In general, dissolution time of stabilizing dissolvable film of the present disclosure may be immediate upon contacting a wet sample collection absorbent. Dissolution time of stabilizing dissolvable film of the present disclosure may also be about 1, 2, 3, 4, 5, 10, 15, 20, 25, 30, 35, 40, 45, 50, or about 55 seconds, or about 1, 2, 3, 4, 5, 6, 7, 8, 9, or 10 minutes or longer. Preferably, dissolution time is about 1, 2, 3, 4, 5, 6, 7, 8, 9, or 10 minutes or longer. More preferably, dissolution time is about 3, 4, 5, 6, 7, or about 8 minutes. Most preferably, dissolution time of a stabilizing dissolvable film of the present disclosure is immediate upon contacting a wet sample collection absorbent.

When a biological sample is contacted with a stabilizing dissolvable film, a stabilizing film may deliver an amount of stabilizing composition sufficient to stabilize a collected sample for a period of about 1, 2, 3, 4, 5, 6, or 7 days, 1, 2, 3, or 4 weeks, or 1, 2, 3, 4, 5, 6, 7, 8, 9, 10, 11, or 12, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or about 35 years or longer under storage and transport conditions normally used with collected biological samples. Storage and transport conditions normally used with collected biological samples may include storage under low light conditions, at ambient room temperature or colder, and under ambient humidity conditions or conditions normally associated with storage in the presence of a desiccant. For instance, storage and transport temperature conditions normally used with collected biological samples may be about 30, 25, 20, 15, 10, or about 5° C. or lower. Additionally, storage and transport humidity conditions normally used with collected biological samples may be about 50, 40, 30, 25, 20, 15, 10, or about 5% humidity or lower. Preferably, storage and transport conditions are storage away from direct sunlight or a source of heat, at a temperature of about 25° C. or colder, and under humidity conditions of about 20% or dryer.

As various changes could be made in the above compositions and methods without departing from the scope of the disclosure, it is intended that all matter contained in the above description and in the Examples given below, shall be interpreted as illustrative and not in a limiting sense.

### EXAMPLES

The following examples illustrate various embodiments of the invention.

### Example 1

### Reagents for Use on Reagent Cassettes for Evidence Sample Stabilization

A method for biological evidence sample stabilization using solutions comprised of EDTA 50 mM/0.1% sodium Azide/0.01% Tween®-20 (EAT) and a solution of EDTA 50 mM/0.01% Tween®-20 (ET) were tested. Samples were either air dried, exposed to a desiccant, or placed into a storage tube without first air drying and without a desiccant.

First, it was determined if the preservative solution of the invention can mitigate the harmful effects of drying a biological evidence specimen in a tube that has no desiccant within the tube. A previous experiment showed there to be beneficial effects of the use of a preservative solution in controlling or preventing bacterial and fungal growth on a biological evidence specimen. This experiment expanded upon that testing.

The materials used are listed in Table 1. It will be appreciated that SecurSwabs are cotton tipped swabs except where another material has been substituted for the cotton tip as noted in the description hereafter.

TABLE 1
Materials for preservative solution analysis.

| Materials | Manufacturers |
|---|---|
| 90 SecurSwabs (Lot # 11041), | Bode Cellmark Forensics, Lorton, VA |
| see FIG. 40 | |
| 45 knitted Polyester Swabs | |
| 25 mL Saliva | |
| QiaSymphony Reagents | Qiagen, Germantown, MD |
| Quantifiler Reagents | Applied Biosystems/Life Technologies, |
| | Grand Island, NY |
| Quantifiler Duo | Applied Biosystems/Life Technologies, |
| | Grand Island, NY |

The controls for the experiment were prepared as follows: 5 aliquots of 100 μL of saliva stored for 14 days at −20° C. (recovery was treated as 100% yield); 5 aliquots of 100 μL of saliva stored open for 14 days at room temperature (RT); each was reconstituted in 100 μL of DNA grade water prior to starting the extraction; and reagent blanks.

The test swabs were prepared as set forth in Table 2, where the experimental design required use of a knitted polyester swab, knitted polyester material was substituted onto a SecurSwab as a replacement for the cotton collector on the swab end. Where indicated the SecurSwab desiccant was removed from the SecurSwab device prior to the addition of saliva.

TABLE 2
Experimental protocol.
Data List A

| | |
|---|---|
| 1 | 5 SecurSwabs with 100 μL of Saliva stored at RT for 14 days |
| 2 | 5 SecurSwabs with 100 μL of Saliva stored at RT for 14 days (Pre-treat swabs |
| | with a 50 mM EDTA/.1% sodium Azide/.01% Tween ®-20 solution prior to |
| | addition of saliva). |
| 3 | 5 SecurSwabs with 100 μL of Saliva stored at RT for 14 days (Pre-treat swabs |
| | with a 50 mM EDTA/.1% sodium Azide/.01% Tween ®-20 solution prior to |
| | addition of saliva. Remove desiccant prior to addition of saliva). |
| 4 | 5 SecurSwabs with 100 μL of Saliva stored at RT for 14 days (Remove |
| | desiccant prior to addition of saliva). |
| 5 | 5 SecurSwabs with 100 μL of Saliva stored at RT for 14 days (Put knitted |
| | polyester swab in SecurSwab format). |
| 6 | 5 SecurSwabs with 100 μL of Saliva stored at RT for 14 days (Pre-treat swabs |
| | with a 50 mM EDTA/.1% sodium Azide/.01% Tween ®-20 solution prior to |
| | addition of saliva). (Put knitted polyester swab in SecurSwab format). |
| 7 | 5 SecurSwabs with 100 μL of Saliva stored at RT for 14 days (Pre-treat swabs |
| | with a 50 mM EDTA/.1% sodium Azide/.01% Tween ®-20 solution prior to |
| | addition of saliva. Remove desiccant prior to addition of saliva). (Put knitted |
| | polyester swab in SecurSwab format). |
| 8 | 5 SecurSwabs with 100 μL of Saliva stored at RT for 14 days (Remove |
| | desiccant prior to addition of saliva). (Put knitted polyester swab in SecurSwab |
| | format). |
| 9 | 5 SecurSwabs with 100 μL of Saliva Air Dried at RT for 14 days |
| 10 | 5 SecurSwabs with 100 μL of Saliva Air Dried at RT for 14 days (Put knitted |
| | polyester swab in SecurSwab format). |
| 11 | 5 SecurSwabs with 100 μL of Saliva Air Dried at RT for 14 days (Pre-treat |
| | swabs with a 50 mM EDTA/.1% sodium Azide/.01% Tween ®-20 solution prior to |
| | addition of saliva). |
| 12 | 5 SecurSwabs with 100 μL of Saliva Air Dried at RT for 14 days (Pre-treat |
| | swabs with a 50 mM EDTA/.1% sodium Azide/.01% Tween ®-20 solution prior to |
| | addition of saliva). (Put knitted polyester swab in SecurSwab format). |
| 13 | 5 SecurSwabs with 100 μL of Saliva Air Dried at RT for 14 days (Pre-treat |
| | swabs with a 50 mM EDTA/.01% Tween ®-20 solution prior to addition of saliva). |
| 14 | 5 SecurSwabs with 100 μL of Saliva Air Dried at RT for 14 days (Pre-treat |
| | swabs with a 50 mM EDTA/.01% Tween ®-20 solution prior to addition of saliva). |
| | (Put knitted polyester swab in SecurSwab format). |
| 15 | 5 SecurSwabs with 100 μL of Saliva stored at RT for 14 days (Pre-treat swabs |
| | with a 50 mM EDTA/.01% Tween ®-20 solution prior to addition of saliva. |
| | Remove desiccant prior to addition of saliva). |
| 16 | 5 SecurSwabs with 100 μL of Saliva stored at RT for 14 days (Pre-treat swabs |
| | with a 50 mM EDTA/.01% Tween ®-20 solution prior to addition of saliva. |
| | Remove desiccant prior to addition of saliva). (Put knitted polyester swab in |
| | SecurSwab format). |
| 17 | 5 SecurSwabs with 100 μL of Saliva stored at RT for 14 days (Pre-treat swabs |
| | with a 50 mM EDTA/.01% Tween ®-20 solution prior to addition of saliva). |
| 18 | 5 SecurSwabs with 100 μL of Saliva stored at RT for 14 days (Pre-treat swabs |
| | with a 50 mM EDTA/.01% Tween ®-20 solution prior to addition of saliva). (Put |
| | knitted polyester swab in SecurSwab format). |

All reagents were thoroughly mixed in the original vials prior to use in the reactions. When a reagent mix was prepared from component solutions, the final solution was thoroughly mixed to obtain a homogeneous solution. The test swabs identified in Data List A above were prepared as follows.

A 50 mM EDTA/0.1% Sodium Azide Solution/0.01% Tween®-20 (EAT solution) and a 50 mM EDTA/0.01% Tween®-20 (ET solution) were made. Thirty SecurSwabs were obtained and 30 knitted polyester swabs were prepared. Each knitted polyester swab was assembled into the SecurSwab format by substituting the knitted polyester for the SecurSwab cotton swab. Each swab was weighed, as was each associated cap used to close the SecurSwab tube holder and the values were recorded.

Each swab was removed from its SecurSwab holder and plunged into the EAT Solution or the ET Solution for 5 seconds according to Data List A (Table 2). The saturated swab and associated cap were weighed and the value recorded. The swab was allowed to dry overnight at RT.

Saliva was aliquoted into individual tubes. Approximately 100 uL of Saliva was pippetted into 100, 2.0 mL tubes. The dried swabs were each weighed along with the associated cap and the values were recorded. Each swab was inserted into a 2.0 mL tube containing saliva for 5 seconds according to Data List A (Table 2). The swabs were each weighed along with the associated cap and the values were recorded. Each swab was stored at the appropriate storage temperature for 14 days.

After 14 days, the swabs were removed from storage conditions and the swab head was snapped or cut head off into a sterilized and labeled 2.0 mL tube. The DNA was extracted using the QIAsymphony following SOP BTF00262 following procedure A provided by the manufacturer: This procedure is suitable for buccal swabs; evidence cuttings; evidence swabs; bloodstain card, FTA, or S&S; and Bode Buccal Collector punches, 500 μL lysis. The samples were incubated for 1.5 hours and the DNA was eluted into 50 μL of TE buffer. SOP BTF00262 Procedure A is a QIAsymphony manufacturer provided protocol which can be modified by the user to suit the particular needs of the specimen and individual user needs and would be determinable by one skilled in the art.

The hDNA was quantified using Quantifiler Human DNA Quantification Kit following the SOP BTF00242 half reaction protocol. (Quantify Standards from QT Duo as well). The hDNA was quantified using Quantifiler Duo Human DNA Quantification Kit following the SOP BTF00242 half reaction protocol. (Quantify Standards from Quantifiler as well).

The swabs were then analyzed for DNA yields and the results analyzed according to the following parameters: compare the DNA yields for each variable set forth in Data List A (Table 2); compare the DNA yields for the knitted polyester vs. cotton; compare the DNA yields for air dry; and, compare the DNA yields for each preservative solution.

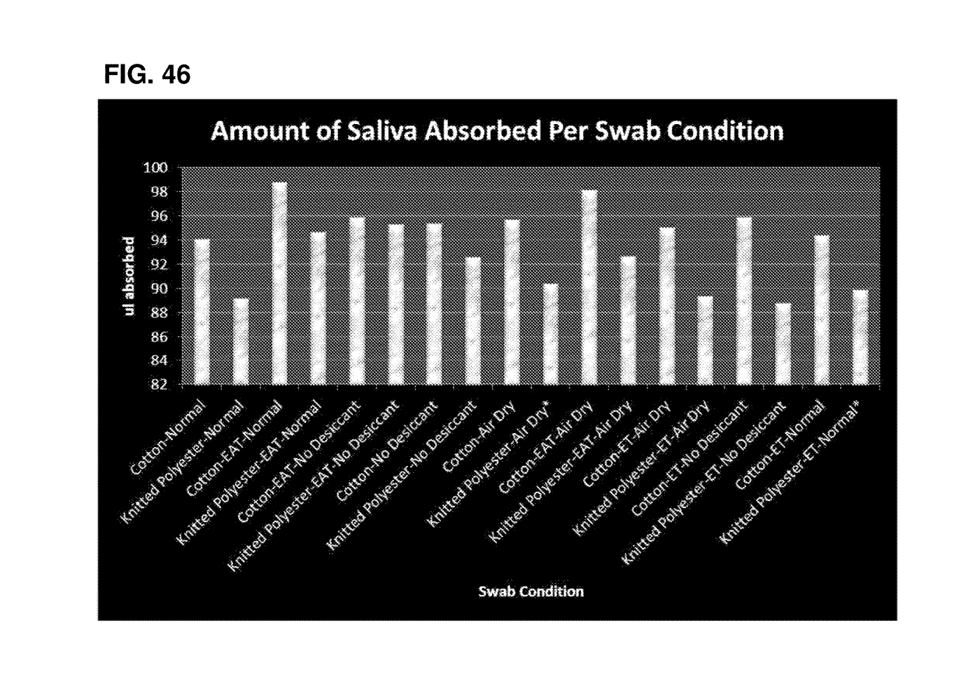

The amount of saliva absorbed by each swab is shown in FIG. 46. The cotton swab having the EAT solution applied thereto presented the greatest amount of saliva absorbed per swab. Further, the replacement of the cotton swab with a knitted polyester tip presented lower values of saliva absorbed per swab.

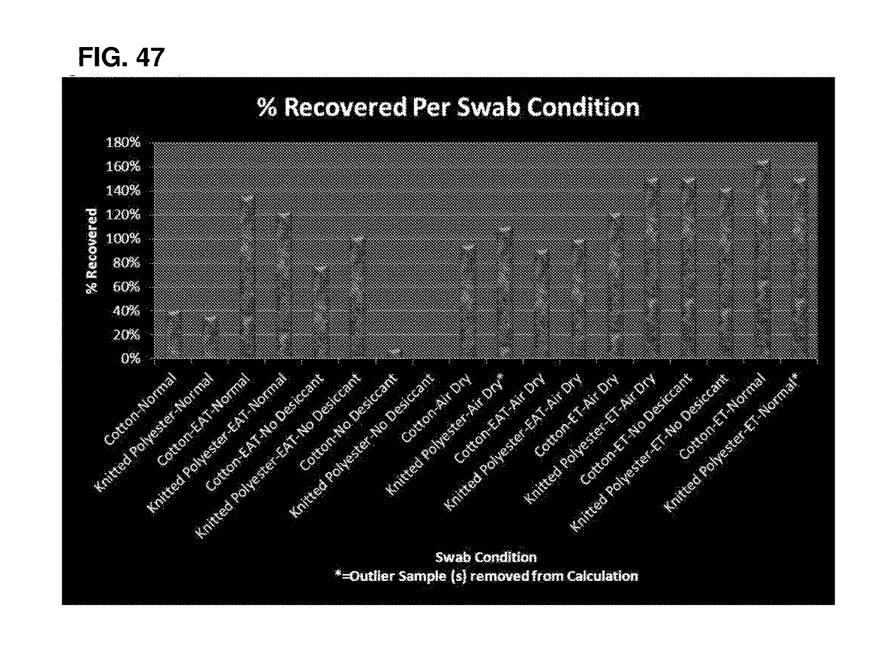

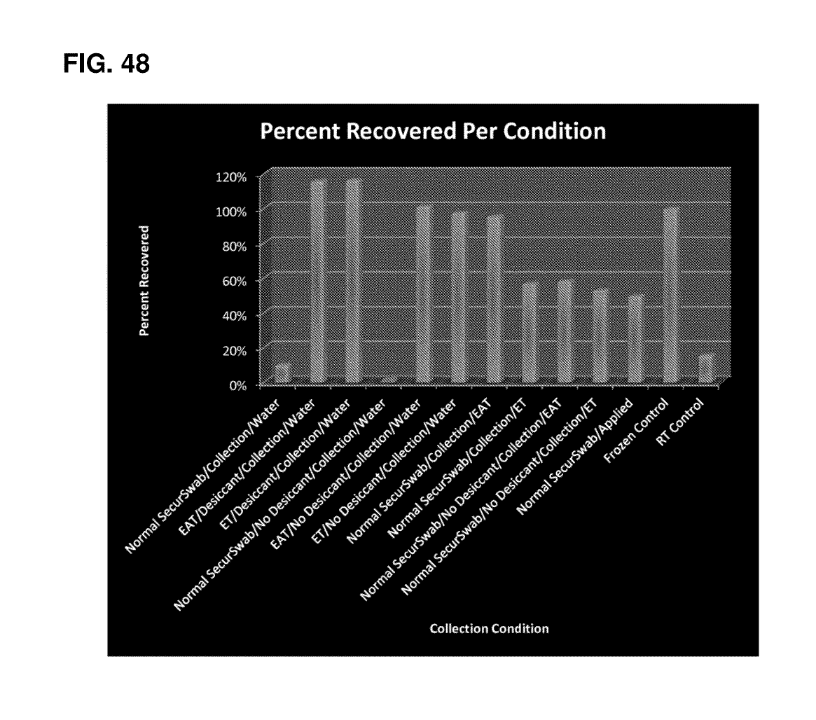

The percentage of DNA recovered using various reagents is shown in FIG. 47. The cotton swab without a desiccant presented the lowest percentage of DNA recovered as compared to the frozen control. The addition of EAT to the normal test swabs (those having a desiccant in the holding tube) produced significantly better DNA yield for both the cotton swab and the knitted polyester tip both with and without desiccant. All samples that were first air-dried had yields of about 90% to about 110%, except the cotton with ET, which had a DNA yield of about 120%. However, the swab that had a knitted polyester tip and received the ET treatment presented the highest DNA yield of the air-dried group. The best overall results were shown by the swabs that received ET and either no desiccant or ET with desiccant in the SecurSwab device holding or storage tube.

In summary, the preservative, dried on the swab prior to insertion, increased the amount of DNA recovered versus a standard swab in the SecurSwab format. Three storage conditions/methods were evaluated in this experiment: air-drying, drying by means of a desiccant, and drying inside a closed system with no desiccant. Within each condition a subset of variables was examined including swab type and two preservative solutions. After being stored for fourteen (n=14) days at room temperature (−20° C. 17% RH) the samples were removed and the swabs were extracted utilizing the Qiagen QiaSymphony Kit. The samples were then quantified using both Quantifiler and Quantifier Duo kits. The average DNA yield from both quants was calculated. Three outlier samples were removed from the calculations and are indicated where appropriate. The DNA yield from the frozen control aliquots will be treated at 1004 and as 100% recovered for the purpose of this study. Percentage recovered results can be found in FIG. 47. These were calculated based off of the expected DNA yield given the average amount of saliva absorbed.

The frozen control aliquots yielded an average quant value of 7.947 ng/μL, which was treated as a 100% recovery for 100 μL of saliva. The room temperature control aliquots yielded an average quant value of 1.3510 ng/μL for a 17% recovery. The SecurSwab collector with the standard cotton swab absorbed on average 94.06 μL of saliva and yielded an average quant value of 3.02 ng/μL for a 40% recovery. The SecurSwab collector with the knitted polyester swab absorbed on average 89.18 μL of saliva and yielded an average quant value of 2.54 ng/μL for a 36% recovery. The SecurSwab collector with the standard cotton swab pre-treated with the EAT solution absorbed on average 98.82 μL of saliva and yielded an average quant value of 10.68 ng/μL for a 136% recovery. The SecurSwab collector with the knitted polyester swab pre-treated with the EAT solution absorbed on average 94.66 μL of saliva and yielded an average quant value of 9.20 ng/μL for a 122% recovery.

The SecurSwab collector with the standard cotton swab pre-treated with the EAT solution and had the desiccant removed absorbed on average 95.88 μL of saliva and yielded an average quant value of 5.94 ng/μL for a 78% recovery. The SecurSwab collector with the knitted polyester swab pre-treated with the EAT solution and had the desiccant removed absorbed on average 95.3 μL of saliva and yielded an average quant value of 7.74 ng/μL for a 102% recovery. The SecurSwab collector with the standard cotton swab and the desiccant removed absorbed on average 95.38 μL of saliva and yielded an average quant value of 0.63 ng/μL for an 8% recovery. The SecurSwab collector with the knitted polyester swab and the desiccant removed absorbed on average 92.62 μL of saliva and yielded an average quant value of 0.05 ng/μL for a 1% recovery. The SecurSwab collector with the standard cotton swab and air-dried absorbed on average 95.7 μL of saliva and yielded an average quant value of 7.24 ng/μL for a 95% recovery.

The SecurSwab collector with the knitted polyester swab and air-dried absorbed on average 90.4 μL of saliva and yielded an average quant value of 7.91 ng/μL for a 110% recovery. This variable had two outliers removed prior to the calculation being performed.

The SecurSwab collector with the standard cotton swab pre-treated with the EAT solution and air-dried absorbed on average 98.18 μL of saliva and yielded an average quant value of 7.12 ng/μL for a 91% recovery. The SecurSwab collector with the knitted polyester swab pre-treated with the EAT solution and air-dried absorbed on average 92.68 μL of saliva and yielded an average quant value of 7.40 ng/μL for a 100% recovery. The SecurSwab collector with the standard cotton swab pre-treated with the ET solution and air-dried absorbed on average 95.04 μL of saliva and yielded an average quant value of 9.20 ng/μL for a 122% recovery. The SecurSwab collector with the knitted polyester swab pre-treated with the ET solution and air-dried absorbed on average 89.36 μL of saliva and yielded an average quant value of 10.72 ng/μL for a 151% recovery. The SecurSwab collector with the standard cotton swab pre-treated with the ET solution and had the desiccant removed absorbed on average 95.88 μL of saliva and yielded an average quant value of 11.50 ng/μL for a 151% recovery.

The SecurSwab collector with the knitted polyester swab pre-treated with the ET solution and had the desiccant removed absorbed on average 88.76 μL of saliva and yielded an average quant value of 10.06 ng/μL for a 143% recovery. The SecurSwab collector with the standard cotton swab pre-treated with the ET solution absorbed on average 94.42 μL of saliva and yielded an average quant value of 12.45 ng/μL for a 166% recovery. The SecurSwab collector with the knitted polyester swab pre-treated with the ET solution absorbed on average 89.875 μL of saliva and yielded an average quant value of 10.80 ng/μL for a 151% recovery. The variable had an outlier removed prior to the calculation being performed.

The first method of storage involved the standard SecurSwab with desiccant. The SecurSwab containing the cotton swab and knitted polyester swab resulted in a percent recovery of 40% and 36%, respectively. This data indicates that the decrease in percent recovery compared to the frozen controls can possibly be explained as retention by the swab during extraction or a breaking down of the DNA during drying and storage, or a combination of the two.

As a worst case scenario, during the collection of a saliva sample, the second method of storage examined a set of five (n=5) SecurSwabs containing both swab types. These samples had the desiccant removed prior to saliva application and storage at room temperature. The result was a dramatic decrease in DNA recovered. The cotton swab recovered 8%, and the knitted polyester swab recovered 1%. The data from these experiments indicate that a wet swab should not be stored in a plastic device without a desiccant or adequate method of drying.

The third method of storage examined the effects of air-drying the sample for fourteen days prior to processing. After saliva application, the swabs were stored open to the air in the laboratory. The cotton and knitted polyester swabs had a percent recovery of 95% and 110%, respectively. This increase in percent recovery as compared to the standard SecurSwab storage method involving a desiccant can be explained in two ways. The previous experiment has shown that the faster a swab dries, the greater the DNA yield, by comparing the IFP and SecurSwab formats. The air drying of a sample containing approximately 100 μL will occur in about four to five hours. As this time frame is similar to the IFP, it is not surprising that the DNA yield was greater than the SecurSwab format which will dry a sample in approximately 24 hours.

The second hypothesis for the increase in DNA yield involves the plastic tube of the SecurSwab device. Oxygen has been shown to aid in healing, possibly being exposed to an abundance of oxygen during the fourteen days prevented the nucleases and other items which break down DNA from having a larger impact. The SecurSwab format utilizes a plastic tube with only two small air holes in the chimera cap. The flow of oxygen in this format is significantly decreased as compared to air drying. Another aspect that must be considered, although the impact if any cannot be quantified at this time, is potential contamination. This study stored the samples open to the air in the laboratory and stopped analysis at the quantification step. Although the area was sectioned off, it is not possible to determine if any of the DNA obtained from the swabs is exogenous, coming from a source other than the saliva applied.

The previous paragraphs discussed the results for un-treated cotton and knitted polyester swabs in three storage conditions. The next variable that will be discussed is the addition of a preservative solution to the swab prior to saliva application. The two preservative solutions are abbreviated as EAT and ET herein.

Using the standard SecurSwab format for storage and pre-treating the swabs with the EAT and ET preservatives resulted in DNA yields greater than the frozen control samples. For swab types, cotton and knitted polyester, the ET preservative yielded—30% more DNA than the EAT solution. This result was not unexpected as the EAT solution in the previous experiment also outperformed the frozen controls.

When the desiccant was removed and the swabs were stored for fourteen days in the plastic tubes, the preservative solutions displayed a great affinity for sample protection. Although the DNA yields decreased compared to having the desiccant present, each swab yielded a comparable amount or outperformed the frozen control DNA yield. In each instance, having the preservatives present increased the percent recovery by a factor of two or four compared to the standard SecurSwab format with un-treated swabs. This was a surprising result as it indicates that in the presence of a preservative solution, the rate of drying has a lesser impact.

The last method of storage involved air-drying the pre-treated swabs at room temperature for fourteen days. The same caveats that were described above with the un-treated swabs stored open to the environment hold true for the treated swabs as well. The EAT preserved swabs were essentially equal to the frozen control samples, while the ET preserved swabs outperformed the frozen controls.

It was frequently observed in this experiment that the experimental test samples outperformed the frozen controls. This was an unexpected result as it was thought that freezing the samples is the best method of storage, although it is not a completely unexplainable result. Freezing the sample should inactivate the DNases and other enzymes/components which aid in the breakdown of DNA. Freezing however is not instantaneous and negative effects can still occur while the sample is freezing. In addition, in order to extract the sample, it must be thawed. This can also impact the cells and DNA. Theoretically, the extraction should be similar between extracting a swab and extracting a liquid substrate. However, this theory has not been fully explored by product development at this time.

The experiment confirmed that drying time directly and significantly impacts resulting DNA yields. These results are supported by the data indicating that the air-dried untreated swabs greatly outperformed the standard SecurSwabs, which greatly outperformed the swabs with the desiccants removed. The standard SecurSwabs also outperformed the room temperature controls. This is further evidence of drying time having a positive impact on DNA yield. The saliva applied to the swab will have dried in approximately 24 hrs while it will take much longer for the 100 μL of saliva to dry in a tube. The increase in drying time allows for a greater breakdown of the DNA.

The results also confirmed that a preservative applied to the swab prior to saliva application will increase the DNA yield. Further, at a fourteen day time period, removal of the desiccant does not have an overly detrimental effect in the presence of a preservative.

This study evaluated two preservative solutions: EAT and ET. The preservative solutions appear to perform best when paired with a desiccant but also performed well air-dried. Air drying poses additional risks and is not really feasible for forensic applications, so a closed desiccated system is ideal. In this study, ET performed slightly better than EAT. This is promising news as the ET preservative is more likely to be safe to be used for buccal collection compared to EAT.

### Example 2

### Effect of Reagent on DNA Yield after Biological Stain Collection

The objective of this analysis was to determine if the reagent containing preservative benefits the DNA yield after the collection of a biological stain. These experiments tested the reagent/preservatives abilities during sample collection.

The materials used are listed in Table 3. It will be appreciated that SecurSwabs are cotton tipped swabs except where another material has been substituted for the SecureSwab cotton swab tip as noted in the description hereafter.

TABLE 3
Materials for preservative solution analysis.

| Materials | Manufacturers |
|---|---|
| 55 SecurSwabs (Lot # 11041), | Bode Cellmark Forensics, Lorton, VA |
| see FIG. 40 | |
| 25 mL Saliva | |
| QiaSymphony Reagents | Qiagen, Germantown, MD |
| Quantifiler Reagents | Applied Biosystems/Life Technologies, |
| | Grand Island, NY |
| Quantifiler Duo | Applied Biosystems/Life Technologies, |
| | Grand Island, NY |

The controls for the experiment were prepared as follows: 5 aliquots of 100 μL of saliva stored for 14 days at −20° C. (recovery was treated as 100% yield); 5 aliquots of 100 μL of saliva stored open for 14 days at room temperature (RT); each was reconstituted in 100 μL of DNA grade water prior to starting the extraction; 5 SecurSwabs with 100 μL of saliva stored for 14 days at RT (used for uncollected controls) and reagent blanks.

The test swabs were prepared as set forth in Table 4. Where the experimental design required the ET and EAT solutions as formulated in Example 1 above. Where indicated the SecurSwab desiccant was removed from the SecurSwab device prior to the addition of saliva.

TABLE 4
Experimental protocol.
Data List B

| | |
|---|---|
| 1 | 5 SecurSwabs used to collect 100 μL of Saliva from a vinyl tile stored at RT for |
| | 14 days. |
| 2 | 5 SecurSwabs used to collect 100 μL of Saliva from a vinyl tile stored at RT for |
| | 14 days (Pre-treat swabs with EAT solution prior to addition of saliva). |
| 3 | 5 SecurSwabs used to collect 100 μL of Saliva from a vinyl tile stored at RT for |
| | 14 days (Pre-treat swabs with ET solution prior to addition of saliva). |
| 4 | 5 SecurSwabs used to collect 100 μL of Saliva from a vinyl tile stored at RT for |
| | 14 days. Remove desiccant prior to addition of saliva. |
| 5 | 5 SecurSwabs used to collect 100 μL of Saliva from a vinyl tile stored at RT for |
| | 14 days (Pre-treat swabs with EAT solution prior to addition of saliva). Remove |
| | desiccant prior to addition of saliva. |
| 6 | 5 SecurSwabs used to collect 100 μL of Saliva from a vinyl tile stored at RT for |
| | 14 days (Pre-treat swabs with ET solution prior to addition of saliva). Remove |
| | desiccant prior to addition of saliva. |
| 7 | 5 SecurSwabs used to collect 100 μL of Saliva from a vinyl tile stored at RT for |
| | 14 days. Use EAT as wetting solution. |
| 8 | 5 SecurSwabs used to collect 100 μL of Saliva from a vinyl tile stored at RT for |
| | 14 days. Use ET as wetting solution. |
| 9 | 5 SecurSwabs used to collect 100 μL of Saliva from a vinyl tile stored at RT for |
| | 14 days. Use EAT as wetting solution. Remove desiccant. |
| 10 | 5 SecurSwabs used to collect 100 μL of Saliva from a vinyl tile stored at RT for |
| | 14 days. Use ET as wetting solution. Remove desiccant. |

All reagents were thoroughly mixed in the original vials prior to use in the reactions. When a reagent mix was prepared from component solutions, the final solution was thoroughly mixed to obtain a homogenous solution.

Fifty (n=50) square vinyl tiles were cut (1.5 inch×1.5 inch) and each weighed. The weight value was recorded. Saliva was pipetted (100 μL) onto a tile. The tile with the saliva on it was weighed and the value was recorded. The tiles were stored at RT for 2 days. Frozen, RT, and un-collected controls were also prepared.

Twenty SecurSwabs were obtained and the weight of each swab and cap was recorded. Each swab was inserted into the EAT or ET solution for 5 seconds according to Table 4. The weight of each wet swab and cap was recorded. The wet swabs were dried overnight at RT.

DNA grade water was pipetted (100 μL) into 30-2.0 mL tubes. EAT (100 μL) was pipetted into 5-2.0 mL tubes. ET was pipetted (100 μL) into 5-2.0 mL tubes. Each swab was inserted into the appropriate 2.0 mL tube for 5 seconds according to Table 4. Next, specimen samples were collected using the swabs. Each swab was swabbed back and forth across a tile from top to bottom. The swab head was rotated and then swabbed back and forth across the tile from bottom to top. The swabs were stored in the appropriate storage temperature for 14 days according to Table 4.

The swabs were removed from storage conditions after 14 days. The swab head was snapped off into a labeled 2.0 mL tube for further analysis. The DNA was extracted using the QIAsymphony following SOP BTF00262 following procedure A provided by the manufacturer: This procedure is suitable for buccal swabs; evidence cuttings; evidence swabs; bloodstain car; FTA; or S&S; and Bode Buccal Collector punches, 500 μL lysis. The samples were incubated for 1.5 hours and the DNA was eluted into 50 μL of TE buffer. SOP BTF00262 Procedure A is a QIAsymphony manufacturer provided protocol which can be modified by the user to suit the particular needs of the specimen and individual user needs and would be determinable by one skilled in the art.

The hDNA was quantified using Quantifiler Human DNA Quantification Kit following the SOP BTF00242 half reaction protocol. (Quantify Standards from QT Duo as well). The hDNA was quantified using Quantifiler Duo Human DNA Quantification Kit following the SOP BTF00242 half reaction protocol. (Quantify Standards from Quantifiler as well).

This analysis was designed to simulate a possible crime scene scenario in which the saliva was dried on a vinyl tile for two days prior to collection. The variables tested included swab pre-treatment and an evaluation of wetting solutions for collection.

Approximately 100 μL of saliva was pipetted on to 1.5 inch×1.5 inch vinyl tiles. Each tile sample was weighed prior to and after saliva application to determine the amount of saliva applied by weight. After two days at room temperature, the vinyl tiles were swabbed and stored for two weeks at room temperature.

After being stored for fourteen (n=14) days at room temperature, the samples were removed and the swabs were extracted utilizing the Qiagen QiaSymphony kit. The samples were then quantified using both Quantifiler and Quantifiler Duo kits. The average DNA yield from both quants was calculated. A reagent blank associated with 10 of the samples quanted at 0.00187 ng/μL in Quantifiler and 0.00457 ng/μL in Quant Duo. The remaining 4 reagent blanks did not yield detectable quantification values in both systems. The DNA yield from the frozen control aliquots were treated as 100 μL and as 100% recovered for the purpose of this study. Percentage recovered results are shown in FIG. 8. These were calculated based off of the expected DNA yield given the average amount of saliva applied to the tile. It assumes that each swab was capable of swabbing and collecting the entire stain deposited.

The frozen control aliquots yielded an average quant value of 12.933 ng/μL which was treated as a 100% recovery for 100 μL of saliva. The room temperature control aliquots yielded an average quant value of 2.018 ng/μL for a 16% recovery. This is a similar recovery rate to the experiment of Example 1.

The SecurSwab collector with the standard cotton swab collected from a vinyl tile with on average 97.04 μL of saliva applied and yielded an average quant value of 1.272 ng/μL for a 10% recovery.

In comparison the SecurSwab collector that had on average 91.44 μL of saliva applied yielded an average quant value of 5.891 ng/μL for a 50% recovery.

The SecurSwab collector with the standard cotton swab pre-treated with EAT collected from a vinyl tile with on average 94.96 μL of saliva applied and yielded an average quant value of 14.236 ng/μL for a 116% recovery.

The SecurSwab collector with the standard cotton swab pre-treated with ET collected from a vinyl tile with on average 97.14 μL of saliva applied and yielded an average quant value of 14.622 ng/μL for a 116% recovery.

The SecurSwab collector with the standard cotton swab and the desiccant removed collected from a vinyl tile with on average 96.74 μL of saliva applied and yielded an average quant value of 0.209 ng/μL for a 2% recovery.

The SecurSwab collector with the standard cotton swab pre-treated with EAT and the desiccant removed collected from a vinyl tile with on average 97.14 μL of saliva applied and yielded an average quant value of 12.741 ng/μL for a 101% recovery.

The SecurSwab collector with the standard cotton swab pre-treated with ET and the desiccant removed collected from a vinyl tile with on average 97.62 μL of saliva applied and yielded an average quant value of 12.329 ng/μL for a 98% recovery. The SecurSwab collector with the standard cotton swab collected from a vinyl tile using EAT as the wetting solution with on average 99.12 μL of saliva applied and yielded an average quant value of 12.251 ng/μL for a 96% recovery.

The SecurSwab collector with the standard cotton swab collected from a vinyl tile using ET as the wetting solution with on average 98.96 μL of saliva applied and yielded an average quant value of 7.274 ng/μL for a 57% recovery.

The SecurSwab collector with the standard cotton swab and the desiccant removed, collected from a vinyl tile using EAT as the wetting solution with on average 98.52 μL of saliva applied and yielded an average quant value of 7.438 ng/μL for a 58% recovery.

The SecurSwab collector with the standard cotton swab and the desiccant removed collected from a vinyl tile using ET as the wetting solution with on average 100.26 μL of saliva applied and yielded an average quant value of 6.882 ng/μL for a 53% recovery.

The first method of storage involved the standard SecurSwab with desiccant. The SecurSwab containing the cotton swab and saliva applied resulted in a percent recovery of 50%. This is a little higher than previous experiments but is still consistent with the results obtained in Example 1 and indicates that the decrease in percent recovery compared to the frozen controls can possibly be explained as retention by the swab during extraction or a breaking down of the DNA during drying and storage, or a combination of the two. In comparison the SecurSwab which was used to collect saliva from a vinyl tile only recovered 10% compared to frozen control. There is the potential that not all the DNA was collected from the surface or that extensive DNA breakdown occurred during the two-day storage. Although this is a possibility, it is unlikely as the pre-treated swabs have a much higher DNA yield and breakdown during two day storage would be equal. It is then hypothesized that rehydrating the cells after drying and then a slow drying process is more detrimental that drying directly on the swab.

Both preservative solutions significantly outperformed the standard SecurSwab regardless of whether a desiccant was present. At two weeks, all pre-treated swabs used to collect saliva from a vinyl tile were essentially equal to the frozen controls.

As a worst case scenario, during the collection of a saliva sample, the second method of storage examined SecurSwabs that had the desiccant removed prior to saliva collection and storage at room temperature. It was previously mentioned that the removal of the desiccant did not have a dramatic impact if the swab was pre-treated. However, if the swab was not pre-treated, the percent recovered was reduced to 2% compared to the frozen controls. Compared to the standard SecurSwab the percent recovered would be 17%. These results are also similar to the results obtained in Example 1.

The third variable examined utilizing the preservatives as the wetting solution instead of water. Compared to the standard SecurSwab and water, the swabs which utilized a preservative wetting solution yielded more DNA. Although it obtained more DNA than water, it yielded less DNA than a pre-treated swab. The pre-treated swab contained more preservative than what was used as the wetting solution so this could be evaluated if needed.

The previous experiments confirmed that drying time directly and significantly impacts resulting DNA yields. This experiment also confirmed the results from the previous experiment that display evidence to the effect that a preservative will increase the DNA yield when dealing with saliva. This experiment went further and showed that the benefits are still effective after the stain has been dried for two days.

This study evaluated two preservative solutions: EAT and ET. The preservative solutions appear to perform best when paired with a desiccant but also performed well without a desiccant present.

### Example 3

### Preserving Collected Specimens for Storage at 4° C. and −20° C.

The objective of this analysis was to determine the most optimal method for preserving saliva for long term storage at 4° C. and −20° C. The SecurSwab stability study was designed to determine whether DNA samples are stable on the SecurSwab over time by performing a real-time study, as well as an accelerated study, using three types of biological fluid commonly collected: blood, semen, and saliva. The saliva sample collected for this stability study was applied to blank SecurSwabs and refrigerated for one week. After one week, aliquots were prepared and stored in the freezer to serve as fresh controls at each testing time point for the stability study. The SecurSwab stability results at 2, 4, 6, and 8 weeks show that there is a consistently lower DNA yield from the fresh saliva controls compared to the real-time and accelerated test samples. Both a 5 day storage trial and a 20 day storage trial were conducted.

For the 5 day storage trial, solutions were prepared and aliquots were stored at both 4° C. and −20° C. for 5 days. About 75 μL of each solution was applied to two (n=2) blank SecurSwabs, air dried overnight, and extracted using the EZ1 BioRobot (SOP BTF00253), eluting into 50 μL of TE. Samples were quantified using Quantifiler (SOP BTF00242, half reaction), normalized to 1.5 ng/μL and amplified with Identifiler (SOP BTF00429) in a 6 μL reaction at 26 cycles. STR fragments were separated using a 3100 Genetic Analyzer (SOP BT00413), and electropherograms were examined using GeneMapper ID software (SOP BT00450). The controls were saliva undiluted and saliva diluted with DNA grade water. The data from this experiment ultimately showed that saliva samples stored for 5 days at 4° C. and −20° C. appeared to be most suitably preserved with 10 mM EDTA in contrast to saliva containing 0.05% Sodium Azide.

For the 20 day storage trial, solutions were prepared and aliquots were stored at both 4° C. and −20° C. for 20 days. About 75 μL of each solution was applied to two (n=2) blank SecurSwabs, air dried for 4 hours, and extracted using the EZ1 BioRobot (SOP BTF00253), eluting into 50 μL of TE. Samples were quantified using Quantifiler (SOP BTF00242, half reaction), normalized to 1.5 ng/μL and amplified with Identifiler (SOP BT00429) in a 6 μL reaction using 26 cycles. STR fragments were separated using a 3100 Genetic Analyzer (SOP BT00413), and electropherograms were examined using GeneMapper ID software (SOP BT00450). The controls were saliva undiluted and saliva diluted with DNA grade water. The data from this experiment ultimately showed that saliva samples stored for 20 days at 4° C. and −20° C. appeared to be most suitably preserved with 10 mM EDTA in contrast to saliva containing 0.05% Sodium Azide.

The materials used are listed in Table 5. It will be appreciated that SecurSwabs are cotton tipped swabs except where another material has been substituted for the SecureSwab cotton swab tip as noted in the description hereafter.

TABLE 5
Materials for preservative for cold storage.

| Materials | Manufacturers |
|---|---|
| SecurSwabs (Lot # 11041) | Bode Cellmark Forensics, Lorton, VA |
| Saliva | |
| 0.5% Sodium Azide | Ricca Chemical Company |
| 0.5M EDTA | Invitrogen, UltraPure pH 8 |
| Saliva solutions A, B, C, D, | |
| and E (stored at 4° C. | |
| and −20° C. for 20 days) | |
| DNA grade water | Fisher Scientific, Pittsburgh, PA |
| 0.5 mL flip cap tubes | |
| Identifiler reagents: | Applied Biosystems/Life Technologies, |
| Identifiler Master Mix, | Grand Island, NY |
| Identifiler Primer, Taq Gold | |
| 9700 Thermacycler #24 | Applied Biosystems/Life Technologies, |
| | Grand Island, NY |
| 3100 Genetic Analyzer #3 | Applied Biosystems/Life Technologies, |
| | Grand Island, NY |

The controls used included the following: solution D (saliva diluted with DNA grade water; and solution E (saliva, undiluted). The test samples included the following: solution A (saliva containing 0.05% Sodium Azide and 10 mM EDTA); solution B (saliva containing 0.05% Sodium Azide); and solution C (saliva containing 10 mM EDTA). The criteria for establishing STR profile completeness was hterozygote alleles ≧75 rfu and homozygote alleles ≧200 rfu.

The reagents were thoroughly mixed in the original vials prior to use in the reactions. When a reagent mix was prepared from component solutions, the final solution was thoroughly mixed to obtain a homogeneous solution.

A tube of each saliva solution from each temperature stored was allowed to thaw. About 75 μL of each biological fluid was applied onto unused SecurSwabs and air dried at room temperature for 4 hours. DNA was extracted from the saliva samples using the EZ1 BioRobot (Qiagen, Germantown, Md.) following the SOP BTF00253 following procedure A, which is suitable for Buccal Swabs; Evidence Cuttings; Evidence Swabs; Blood on Stain Card; FTA; or S&S; Bode Buccal Collector Punches, 500 μL lysis (step 4.1.19). The samples were incubated for 2 hours and the DNA was eluted into 50 μL of TE buffer. The hDNA was quantified using Quantifiler Human DNA Quantification Kit following the SOP BTF00242 half reaction protocol. The sample extracts were normalized to 1.5 ng/μL for the Identifiler amplification reaction. About 1 μL of the 1.5 ng/μL extract was amplified in a 6 μL Identifiler amplification reaction according to SOP BT00429 for 26 cycles. About 1.5 ng of 9947 was added for the amp positive. The SOP BT00413 protocol was followed to separate STR fragments on a 3100 Genetic Analyzer (Applied Biosystems/Life Technologies, Grand Island, N.Y.). About 0.8 μL of the amplicon product was used and 10 μL of Hi-Di formamide containing 0.20 μL of GS-500LIZ was used. The electropherograms were analyzed using Genemapper software (SOP BT00450) (GeneMapper ID v 3.2.1, Applied Biosystems, Grand Island, N.Y.).

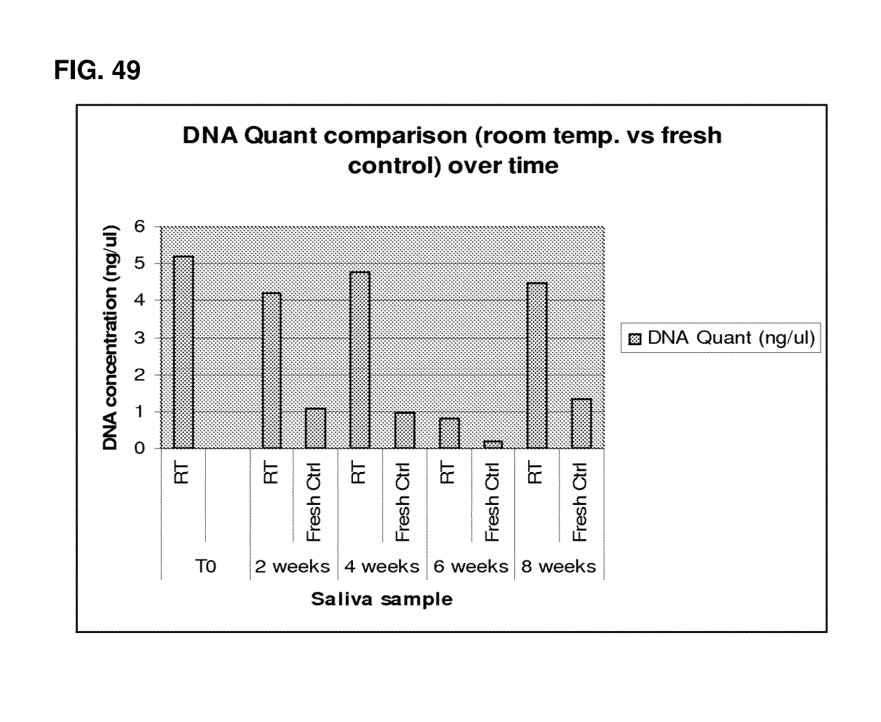

During the set up for the SecurSwab stability experiment, the original saliva sample collected was applied to blank SecurSwabs as test samples and the remainder of the sample solution was refrigerated for one week. Aliquots of the saliva sample were then prepared and stored at −20° C. At each testing time point for the SecurSwab stability study, an aliquot of the saliva sample was removed from the freezer, allowed to thaw, applied to blank SecurSwabs, and allowed to completely dry in order to serve as a fresh control. The data (FIG. 49) shows that the DNA yields for the fresh controls remain consistently lower than the test samples stored at room temperature. The data after storage for 20 days is also summarized in Table 6. From the previous saliva preservation experiment, it was concluded that saliva containing 10 mM EDTA worked better to preserve the sample than saliva containing 0.05% Sodium Azide. The current experiment was repeated to determine if similar results were obtained.

TABLE 6
Data after storage for 20 days.

| | | DNA | | pHT | pHT | |
|---|---|---|---|---|---|---|
| | | conc | DNA | at D8 | at CSF | CSF/ |
| Sample | N | ng/μL | Profile | (rfus) | (rfus) | D8 |
| Saliva A 4° C. | 2 | 10.55 | Complete | 1481 | 1011.5 | 68% |
| Saliva A −20° C. | 2 | 13.40 | Complete | 2197 | 2462 | 112% |
| Saliva B 4° C. | 2 | 1.31 | Poor/complete | 757 | 564.5 | 41% |
| Saliva B −20° C. | 2 | 17.33 | **weak/ | 812 | 743 | 81% |
| | | | complete | | | |
| Saliva C 4° C. | 2 | 18.71 | Complete | 862 | 648.5 | 75% |
| Saliva C −20° C. | 2 | 18.54 | Complete | 1803 | 2049.5 | 113% |
| Saliva D 4° C. | 2 | 2.99 | Complete/weak | 1088 | 853 | 77% |
| Saliva D −20° C. | 2 | 20.43 | Complete/weak | 860 | 727 | 84% |
| Saliva E 4° C. | 2 | 3.37 | Complete | 899 | 907 | 100% |
| Saliva E −20° C. | 2 | 17.80 | Complete | 1262 | 880 | 70% |

*based on quantification;
**weak hDNA profile observed with low ILS ~140 RFUs; Solution A: Saliva containing 0.05% Sodium Azide and 10 mM EDTA; Solution B: Saliva containing 0.05% Sodium Azide; Solution C: Saliva containing 10 mM EDTA; Solution D: Saliva diluted with DNA grade water; Solution E: Saliva, undiluted.

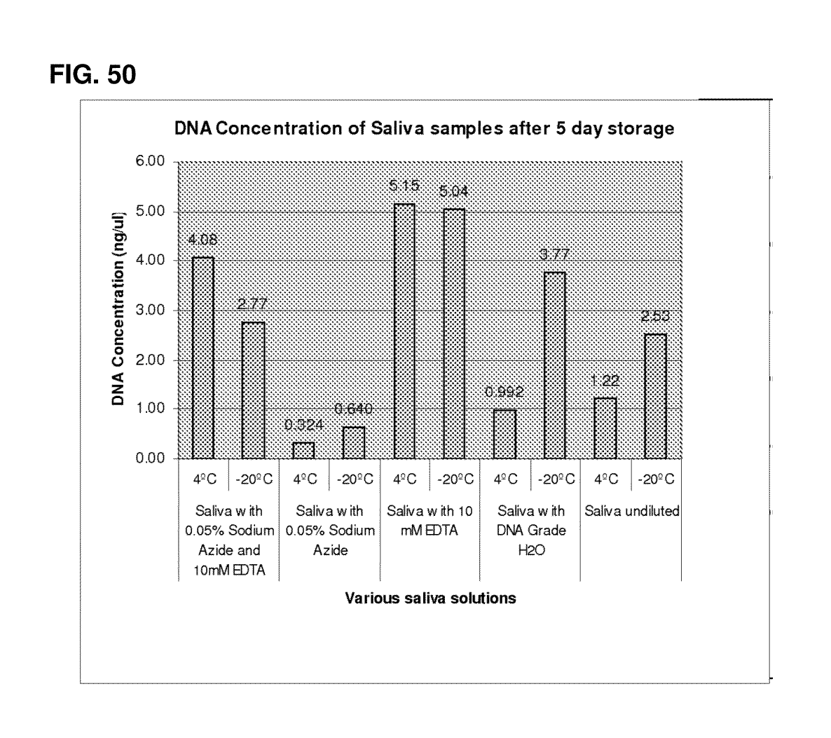

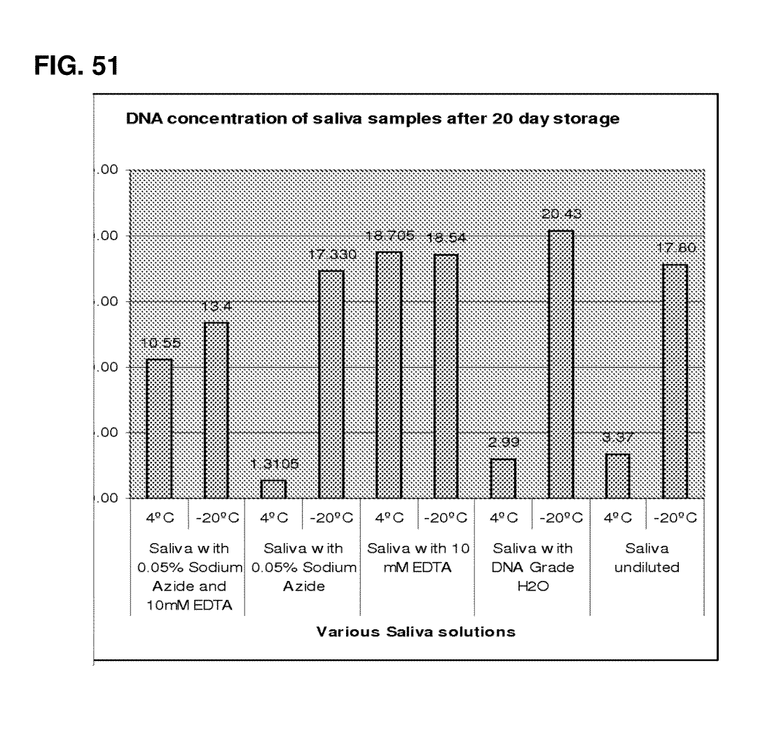

In reviewing the overall quantification data from the previous experiment, the DNA yields were lower than expected. The data from the current experiment shows that the DNA yields across all saliva solutions are much higher than those observed previously (FIG. 50 and FIG. 51). Most of the saliva samples stored at 4° C. fall within the expected range of 1-10 ng/ul. However, all of the samples stored in the freezer were outside of this range. In contrast to the previous experiment, the saliva solution containing 0.05% Sodium Azide only and stored at −20° C. yielded a DNA concentration similar to that of the frozen undiluted saliva. The data does maintain that frozen storage is preferable over refrigeration. It is also consistent that saliva containing 10 mM EDTA generates an even yield of DNA with storage at both 4° C. and −20° C.

With regards to the fresh saliva controls prepared for the SecurSwab stability study, it seems as though the samples would have been preserved much better by freezing at −20° C. alone (either undiluted or diluted with DNA Grade Water) post collection. If refrigeration up to 20 days is desired, it is best to preserve a saliva sample with 10 mM EDTA or a mixture of 0.05% Sodium Azide and 10 mM EDTA.

### Example 4

### Optimization of Preservation Method for Storage at 4° C. and −20° C.

Using the methods and materials of Example 3, the optimal storage method was optimized. All reagents were thoroughly mixed in the original vials prior to use in the reactions. When a reagent mix was prepared from component solutions, the final solution was thoroughly mixed to obtain a homogeneous solution.

Individual 2.0 mL tubes were prepared as provided in Table 7. Each tube was prepared containing 1500 μL solutions each.

TABLE 7
Solution preparation table.

| | Solution A | | | | |
|---|---|---|---|---|---|
| | Saliva with | Solution B | | Solution D | |
| | 0.05% Sodium | Saliva with | Solution C | Saliva with | Solution E |
| | Azide and | 0.05% Sodium | Saliva with | DNA Grade | Saliva |
| Reagent | 10 mM EDTA | Azide | 10 mM EDTA | water | undiluted |
| Saliva | 1320 μL | 1320 μL | 1320 μL | 1320 μL | 1500 μL |
| | fresh saliva | fresh saliva | fresh saliva | fresh saliva | fresh saliva |
| EDTA | 30 μL 0.5M | X | 30 μL 0.5M | X | |
| | EDTA | | EDTA | | |
| Sodium | 150 μL | 150 μL | X | X | |
| Azide | 0.5% Sodium | 0.5% Sodium | | | |
| | Azide | Azide | | | |
| DNA grade | X | 30 μL | | 180 μL | |
| water | | DNA Grade | | DNA grade | |
| | | water | | water | |

About 175 μL of each solution was dispensed into six individual 0.5 mL flip cap tubes. From each set of solutions, 3 tubes were stored at 4° C. and 3 tubes were stored at −20° C. for 5 days. On the 5^{th} day, one tube of each solution from each temperature stored was removed. Frozen samples were allowed to thaw. About 75 μL of each biological fluid was applied onto unused SecurSwabs and air dried overnight at room temperature. DNA was extracted from the saliva samples using the EZ1 BioRobot (Qiagen, Germantown, Md.) following the SOP BTF00253 following procedure A, which is suitable for Buccal Swabs; Evidence Cuttings; Evidence Swabs; Blood on Stain Card; FTA; or S&S; Bode Buccal Collector Punches, 500 μL lysis (step 4.1.19). The samples were incubated for 2 hours and the DNA was eluted into 50 μL of TE buffer. The hDNA was quantified using Quantifiler Human DNA Quantification Kit following the SOP BTF00242 half reaction protocol. The sample extracts were normalized to 1.5 ng/μL for the Identifiler amplification reaction. About 1 μL of the 1.5 ng/μL extract was amplified in a 6 μL Identifiler amplification reaction according to SOP BT00429 for 26 cycles. About 1.5 ng of 9947 was added for the amp positive. The SOP BT00413 protocol was followed to separate STR fragments on a 3100 Genetic Analyzer (Applied Biosystems/Life Technologies, Grand Island, N.Y.). About 0.8 μL of the amplicon product was used and 10 μL of Hi-Di formamide containing 0.20 μL of GS-500LIZ was used. The electropherograms were analyzed using Genemapper software (SOP BT00450) (GeneMapper ID v 3.2.1, Applied Biosystems, Grand Island, N.Y.).

TABLE 8
Data after storage up to 6 weeks.

| | | DNA | | pHT | pHT | |
|---|---|---|---|---|---|---|
| | | conc | DNA | at D8 | at CSF | CSF/ |
| Solution | Sample | ng/μL | Profile | (rfus) | (rfus) | D8 |
| Saliva with | Saliva | 4.08 | Complete | 5301 | 2962 | 54% |
| 0.05% | A 4° C. | | | | | |
| Sodium | Saliva | 2.77 | Complete | 5750 | 3859 | 68% |
| Azide and | A −20° C. | | | | | |
| 10 mM EDTA | | | | | | |
| Saliva with | Saliva | 0.324 | Complete | 3496 | 1964 | 56% |
| 0.05% | B 4° C. | | | | | |
| Sodium | Saliva | 0.640 | Complete | 3065 | 1686 | 54% |
| Azide | B −20° C. | | | | | |
| Saliva with | Saliva | 5.15 | Complete | 4010 | 2735 | 68% |
| 10 mM EDTA | C 4° C. | | | | | |
| | Saliva | 5.04 | Complete | 5593 | 4364 | 78% |
| | C −20° C. | | | | | |
| Saliva with | Saliva | 0.992 | Complete | 4600 | 3473 | 77% |
| DNA grade | D 4° C. | | | | | |
| water | Saliva | 3.77 | Complete | 4377 | 2732 | 62% |
| | D −20° C. | | | | | |
| Saliva | Saliva | 1.22 | Complete | 3308 | 2569 | 81% |
| undiluted | E 4° C. | | | | | |
| | Saliva | 2.53 | Complete | 5096 | 3368 | 66% |
| | E −20° C. | | | | | |

Two preservatives were used for this study: 0.05% Sodium Azide and 10 mM EDTA. Sodium Azide is a preservative capable of preventing bacterial growth. EDTA is a chemical that binds magnesium ions, which are required in order for nucleases to break down DNA molecules. The data shows that saliva containing solely 0.05% Sodium Azide generated the lowest DNA yield when compared to the control solutions D and E (FIG. 53). In contrast, saliva containing solely 10 mM EDTA generated the highest DNA yield compared to the control solutions D and E (FIG. 53). This finding indicates that suppression of nuclease activity by EDTA is more effective in preserving the DNA than prevention of bacterial growth by Sodium Azide.

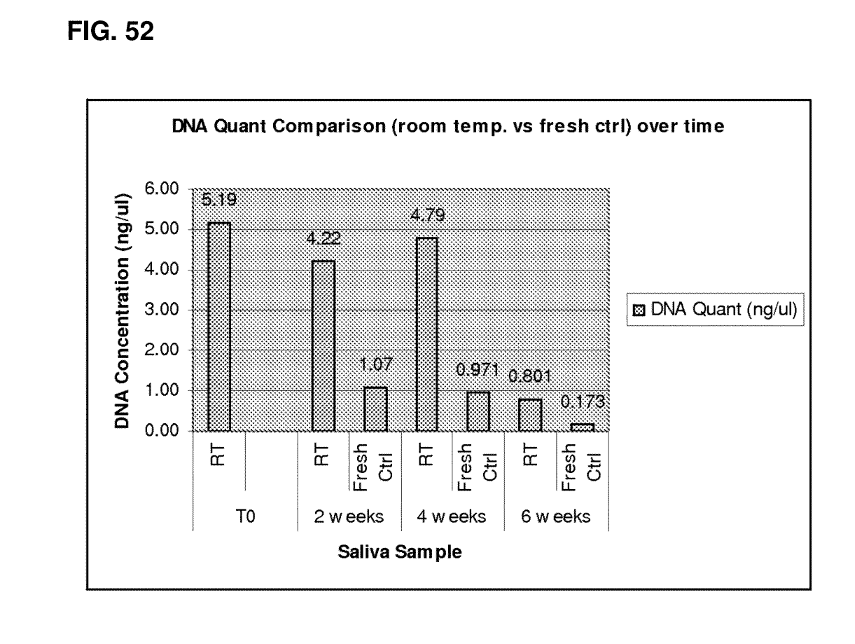

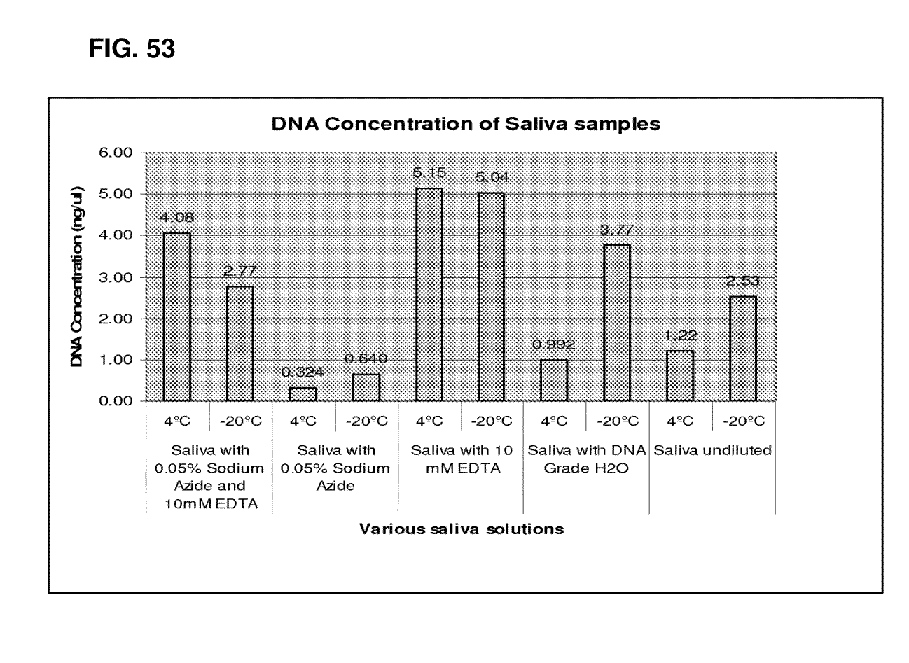

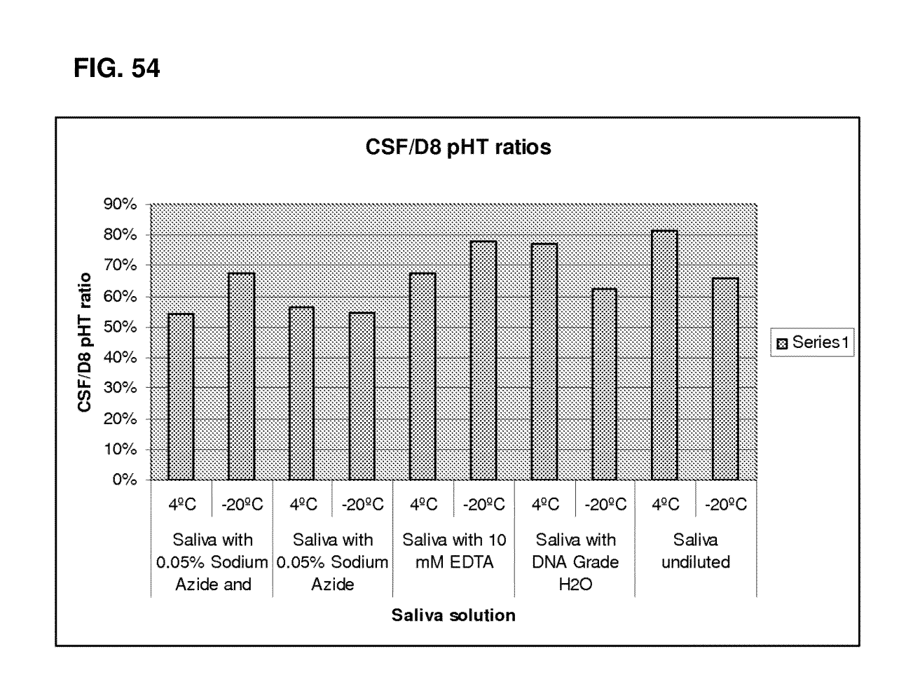

During the set up for the SecurSwab stability experiment (RD2010-13b), the saliva sample was collected from a volunteer and test swabs were prepared by applying 75 μL of the fresh saliva to each SecurSwab used for the study. The remainder of the saliva sample was then stored in the refrigerator for approximately one week. After one week of refrigeration, aliquots of the saliva sample were prepared and stored at −20° C. At each testing time point for the SecurSwab stability study, an aliquot of each biological fluid was removed from the freezer and allowed to thaw. Each aliquot was sufficient enough to apply 75 μL of biological fluid to two (n=2) blank SecurSwabs. These SecurSwabs were allowed to completely dry overnight and served as fresh controls for the test samples of the study. The data (FIGS. 52-54) shows that the DNA yield for the fresh controls was consistently lower than the test samples stored at room temperature. It is believed that the nucleases in the refrigerated saliva sample effectively degraded the DNA, so when it was processed, a lower DNA yield was observed. Saliva samples stored for 5 days at 4° C. and −20° C. were most suitably preserved with 10 mM EDTA in contrast to saliva containing 0.05% Sodium Azide. The data after storage for 6 weeks is also summarized in Table 8.

### Example 5

### Analysis of EAT and ET Preservative Solutions after 30 Day Storage

The effects of both EAT and ET preservative solutions after specimen application and storage for thirty days were evaluated. The materials used are provided in Table 9, the controls are provided in Table 10, and the test samples are provided in Table 11.

TABLE 9
Materials and methods.

| Materials | Manufacturers |
|---|---|
| 90 SecurSwabs (Lot # 11041) | Bode Cellmark Forensics, Lorton, VA |
| 25 mL Saliva | |
| QiaSymphony Reagents | Qiagen, Germantown, MD |
| Quantifiler Reagents | Applied Biosystems/Life Technologies, |
| | Grand Island, NY |
| Quantifiler Duo | Applied Biosystems/Life Technologies, |
| | Grand Island, NY |
| Quantifiler Duo | Applied Biosystems/Life Technologies, |
| | Grand Island, NY |

TABLE 10
Control samples.

| Control | |
|---|---|
| Sample | Description |
| 1 | 5 aliquots of 100 μL of saliva stored for 30 days at −20° C. (recovery |
| | treated as 100% yield) |
| 2 | 5 aliquots of 28.6 μL of saliva stored for 30 days at −20° C. (71.5 μL of |
| | EAT added) |
| 3 | 5 aliquots of 28.6 μL of saliva stored for 30 days at −20° C. (71.5 μL of |
| | ET added) |
| 4 | 5 aliquots of 28.6 μL of saliva stored for 30 days at −20° C. (71.5 μL of |
| | water added) |
| 5 | 5 aliquots of 100 μL of saliva stored open for 30 days at RT (to be |
| | reconstituted in 100 μL of DNA grade water prior to starting the |
| | extraction) |
| 6 | 5 aliquots of 28.6 μL of saliva stored open for 30 days at RT (71.5 μL of |
| | EAT added. Was reconstituted in 100 μL of DNA grade water prior to |
| | starting the extraction). |
| 7 | 5 aliquots of 28.6 μL of saliva stored open for 30 days at RT (71.5 μL of |
| | ET added. Was reconstituted in 100 μL of DNA grade water prior to |
| | starting the extraction). |
| 8 | 5 aliquots of 28.6 μL of saliva stored open for 30 days at RT (71.5 μL of |
| | water added. Was reconstituted in 100 μL of DNA grade water prior to |
| | starting the extraction). |
| 9 | 5 aliquots of 100 μl of saliva stored for 30 days at 56° C. (recovery |
| | treated as 100% yield) |
| 10 | 5 aliquots of 28.6 μL of saliva stored for 30 days at 56° C. (71.5 μL of |
| | EAT added) |
| 11 | 5 aliquots of 28.6 μL of saliva stored for 30 days at 56° C. (71.5 μL of |
| | ET added) |
| 12 | 5 aliquots of 28.6 μL of saliva stored for 30 days at 56° C. (71.5 μL of |
| | water added) |
| 13 | Reagent Blanks |

TABLE 11
Control samples.

| Test | |
|---|---|
| Sam- | |
| ple | Description |
| 1 | 5 SecurSwabs with of 100 μL of saliva stored at RT for 30 days |
| 2 | 5 SecurSwabs with of 100 μL of saliva stored at RT for 30 days |
| | (Pre-treat swabs with EAT solution prior to addition of saliva) |
| 3 | 5 SecurSwabs with of 100 μL of saliva stored at RT for 30 days |
| | (Pre-treat swabs with ET solution prior to addition of saliva) |
| 4 | 5 SecurSwabs with of 100 μL of saliva stored at RT for 30 days. |
| | Remove desiccant prior to addition of saliva. |
| 5 | 5 SecurSwabs with of 100 μL of saliva stored at RT for 30 days |
| | (Pre-treat swabs with EAT solution prior to addition of saliva). |
| | Remove desiccant prior to addition of saliva. |
| 6 | 5 SecurSwabs with of 100 μL of saliva stored at RT for 30 days |
| | (Pre-treat swabs with ET solution prior to addition of saliva). |
| | Remove desiccant prior to addition of saliva. |

The directions for saliva application for 30 day, 60 day, and 6 month samples follow. The EAT and ET solutions were made as provided in Example 1 above. Sixty SecurSwabs and the associated cap were each weighed and the value recorded. Each swab was removed from its device and inserted into the EAT or ET solution for 5 seconds according to Table 10 and Table 11. Each swab and cap was weighed again and the value was recorded. Each swab was allowed to dry over night at RT. About 100 μL of saliva was pipetted into 225 2.0 mL tubes. Each dried swab and cap was weighed and the value was recorded. Then, each swab was inserted into a 2.0 mL tube for 5 seconds according to Table 10 and Table 11. Each swab and cap was weighed and the value recorded. Each swab was stored in the appropriate storage temperature for 30 or 60 days, or 6 months. Swabs were removed from storage conditions and the swab head was snapped off into a labeled 2.0 mL tube.

DNA was extracted from the saliva samples using the QIAsymphony kit following SOP BTF00262 and procedure A, which is suitable for Buccal Swabs; Evidence Cuttings; Evidence Swabs; Blood on Stain Card; FTA; or S&S; Bode Buccal Collector Punches, 500 μL lysis. The samples were incubated for 1.5 hours and the DNA was eluted into 50 μL of TE buffer. The hDNA was quantified using Quantifiler Human DNA Quantification Kit following the SOP BTF242 half reaction protocol. The hDNA was quantified using Quantifiler Duo Human DNA Quantification Kit following SOPBTF00242 half reaction protocol.

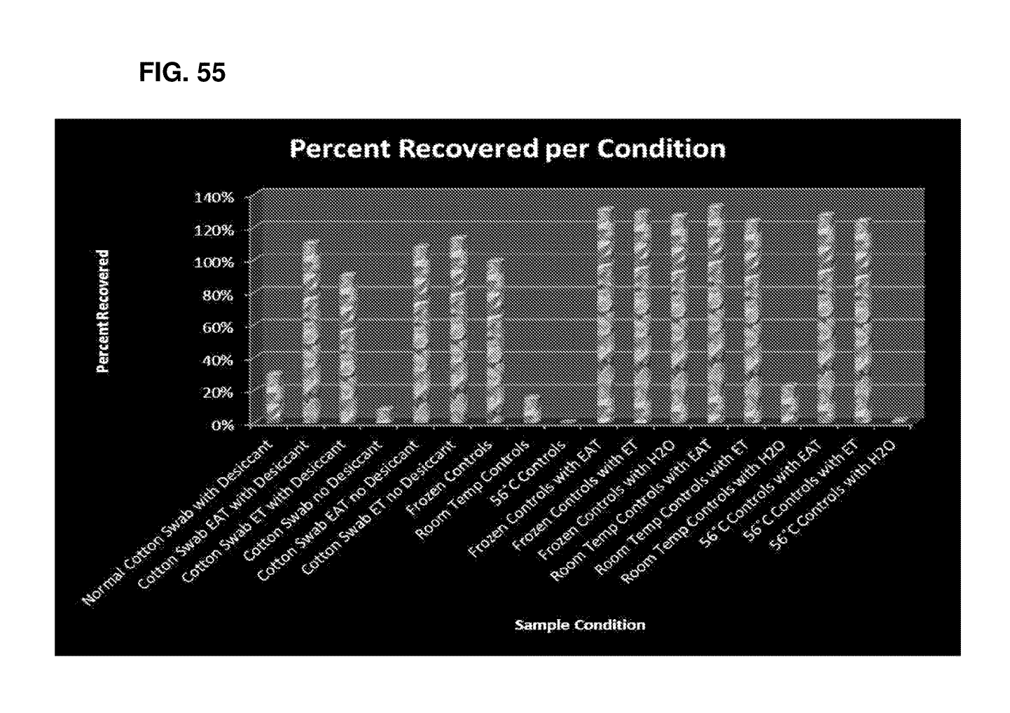

This Example was designed to continue the evaluations of Examples 1-4, expanding the storage time to 30 days, 60 days, and 6 months. After storage for the designated time and temperature, samples were removed and the swabs were extracted from utilizing the Qiagen QiaSymphony. The samples were then quantified using both Quantifiler and Quantifiler Duo. The average DNA yield from both quants was calculated. The DNA yield from the frozen control aliquots will be treated as 100 μL and as 100% recovered for the purpose of this study. Percentage recovered results are shown in FIG. 55. These were calculated based off of the expected DNA yield given the average amount of saliva absorbed.

The frozen control aliquots yielded an average quant value of 8.807 ng/μL which was treated as a 100% recovery for 100 μL of saliva.

The room temperature control aliquots yielded an average quant value of 1.426 ng/μL for a 16% recovery. The 56° C. control aliquots yielded an average quant value of 0.03703 ng/μL for a <1% recovery. The SecurSwab collector with the standard cotton swab absorbed on average 94.62 μL of saliva and yielded an average quant value of 2.573 ng/μL for a 31% recovery. The SecurSwab collector with the standard cotton swab and the desiccant removed absorbed on average 95.18 μL of saliva and yielded an average quant value of 0.7667 ng/μL for a 9% recovery. This result supports the conclusion that drying time significantly impacts DNA yield. The SecurSwab collector with the standard cotton swab pre-treated with the EAT solution absorbed on average 95.56 μL of saliva and yielded an average quant value of 9.383 ng/μL for a 111% recovery. The SecurSwab collector with the standard cotton swab pre-treated with the EAT solution and had the desiccant removed absorbed on average 94.84 μL of saliva and yielded an average quant value of 9.073 ng/μL for a 109% recovery. The SecurSwab collector with the standard cotton swab pre-treated with the ET solution absorbed on average 95.8 μL of saliva and yielded an average quant value of 7.73 ng/μL for a 92% recovery. The SecurSwab collector with the standard cotton swab pre-treated with the ET solution and had the desiccant removed absorbed an average 95.94 μL of saliva and yielded an average quant value of 9.652 ng/μL for a 114% recovery.

This experiment was designed to evaluate the beneficial effects of the preservative solutions in comparison to a standard cotton swab in the SecurSwab device. Compared to the frozen controls, the standard cotton swab had a 31% recovery while the pre-treated swabs yielded over twice as much DNA and were essentially equal to the frozen controls after a thirty day storage period. Even when the desiccant was removed from the system the pre-treated swabs yielded results in line with the frozen controls while the un-treated cotton swabs dropped to a 9% recovery. The previous experiments have shown the beneficial effects of the preservatives with a 14 day storage period. The data from this experiment suggests that the most important time for preservation occurs during the initial drying period, and as storage time increases the sample should remain stable in the presence of the preservative.

Additional control samples were set up for three different storage temperatures (−20° C., room temperature, and 56° C.) using 28.6 μL of saliva and 71.5 μL of either preservative or water. This ratio of 1:2.5 is similar to the ratio of saliva to preservatives when the swab is pre-treated.

The frozen control samples with the addition of EAT yielded an average quant value of 3.319 ng/μL for a 132% recovery. The frozen control samples with the addition of ET yielded an average quant value of 3.279 ng/μL for a 130% recovery. The frozen control samples with the addition of water yielded an average quant value of 3.223 ng/μL for a 128% recovery.

The frozen control samples all yielded percent recoveries greater than 100% of the 100 μL frozen control. There is not a difference between the preservative solutions and water at this temperature. One reason for the non-difference is that at a freezing temperature the nucleases and other enzymes which break down DNA will be inactivated, thus mitigating the beneficial effects. Since the controls contain less saliva (28.6 μL vs. 100 μL), these samples had the nucleases and other harmful components diluted relative to the standard control concentrations.

The room temperature control samples with the addition of EAT yielded an average quant value of 3.364 ng/μL for a 133% recovery. The room temperature control samples with the addition of ET yielded an average quant value of 3.139 ng/μL for a 125% recovery. The room temperature control samples with the addition of water yielded an average quant value of 0.5946 ng/μL for a 24% recovery.

The room temperature samples display results nearly equal the frozen controls samples for the preserved solutions, while the water solution has decreased 5-fold. This shows that at room temperature storage, a preservative is more advantageous than water. The two preservative solutions also yielded twice as much DNA as the standard room temperature controls even though each sample contains about 70% less saliva.

The 56° C. control aliquots with the addition of EAT yielded an average quant value of 3.235 ng/μL for a 128% recovery. The 56° C. control aliquots with the addition of ET yielded an average quant value of 3.144 ng/μL for a 125% recovery. The 56° C. control aliquots with the addition of water yielded an average quant value of 0.04833 ng/μL for a 2% recovery.

At 56° C., the preservative solutions displayed results similar to the frozen and room temperature samples, while the water decreased significantly down to a 2% recovery. Without a preservative solution present, the sample is nearly reduced to nothing at this temperature. It may be beneficial to amplify the frozen, room temperature, and 56° C. controls after normalizing to see the resulting profile. Although the quantification values were similar, the resulting profile may differ due to each storage condition.

This experiment confirmed the results from the previous experiments that display evidence to the effect that a preservative applied to the swab prior to saliva application will increase the DNA yield. This experiment went further and showed that at a thirty-day time period, removal of the desiccant did not have a detrimental effect in the presence of a preservative.

This study evaluated two preservative solutions: EAT and ET. The preservative solutions in past experiments appeared to perform best when paired with a desiccant. In this experiment EAT was essentially equal when used with or without a desiccant while ET worked better without a desiccant.

The results from this experiment show that having the preservative present increases the DNA yield as compared to the frozen control. One additional theory on this could be potential positive effects of the preservative on the samples and substrate. Having the detergent component of the preservatives present on the swab for collection may help to loosen the fibers of the swab, allowing for a greater amount of DNA to be released during extraction. The pre-treatment of the swabs, although the swab is dry prior to saliva application, may prevent the saliva that is absorbed from fully entering the swab, becoming trapped, and not being released during extraction and utilized for analysis.

### Example 6

### Dissolvable Preservative Film

A dissolvable film was created having the characteristics of slowing down specimen degradation, preserving biomolecule contents of collected specimens, specimen penetration, and hands-free application. This dissolvable film, upon contact with a wet collection absorbent, dissolves and percolates through the sample collected on the paper absorbent.

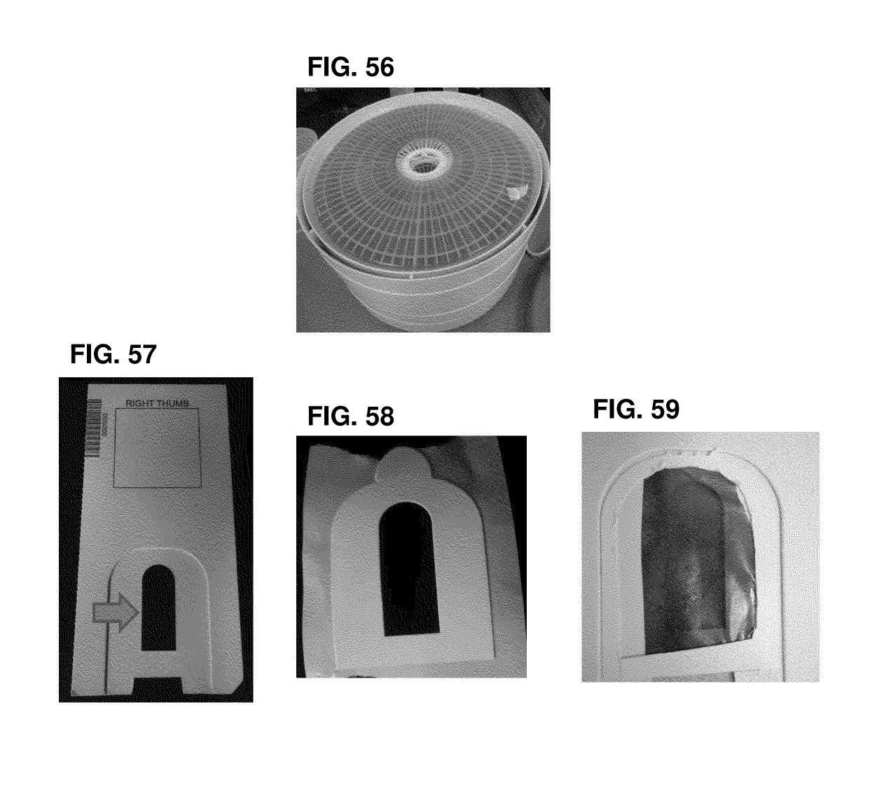

The dissolvable film was created by making a 5% solution of polyvinylpyrrolidone (PVP) in PROTECT solution (Table 12). Also, a 0.25%-0.5% solution of carboxy methyl cellulose (CMC) in PROTECT solution was made. For both solutions, the PVP or CMC powders were slowly added to the PROTECT solution and stirred to ensure that all powder was in the liquid and not on top of or on the sides of the beaker. The PVP and CMC were allowed to dissolve with occasional stirring. After the powder dissolved, the respective beakers were sealed with parafilm. The beakers were inverted 10 times to ensure that none of the PROTECT components settled or fell out of the solution. A desired amount of 5% PVP/PROTECT or 0.25% CMC was poured or pipetted onto a plastic bag placed inside a desiccant chamber. Alternatively, the CMC and PVP solutions may be added to a food dehydrator (FIG. 56). The liquid was allowed to dry, creating a film that could be manipulated.

TABLE 12
PROTECT solution.

| PROTECT | pH: 8.31 | |
|---|---|---|
| Reagent | Concentration | |
| EDTA | 10 | mM |
| Sodium Azide | 0.05% | |
| Tween ®-20 | 0.01% | |
| EGTA | 2.5 | mM |
| KCl | 25 | mM |
| Tris-HCl | 50 | mM |
| SDS | 5.83 | mM |

| | |
|---|---|
| Blue Food Coloring | 10 mLs per liter |

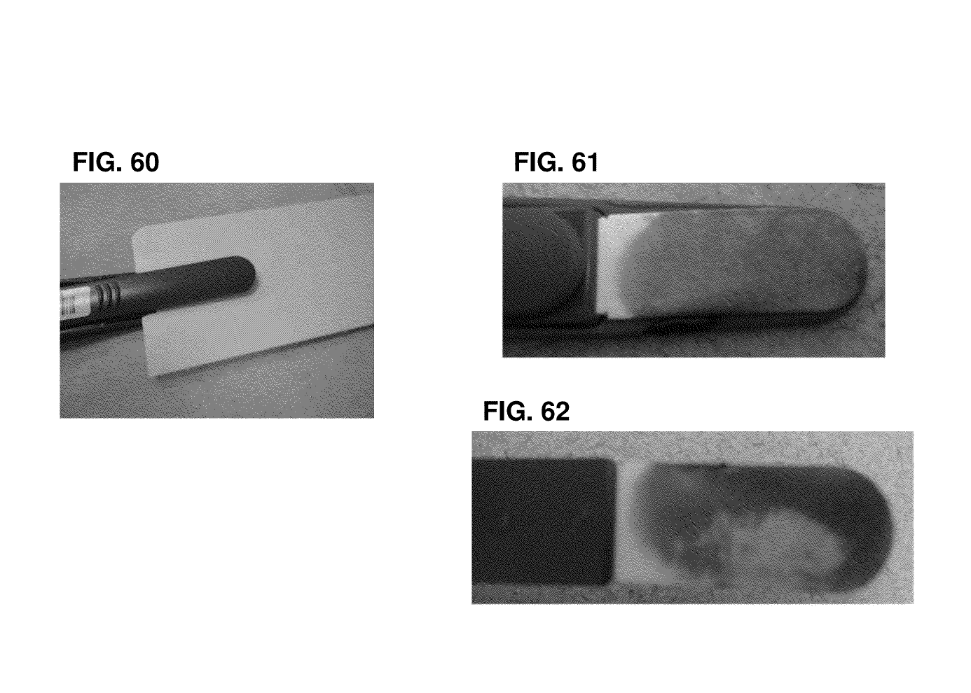

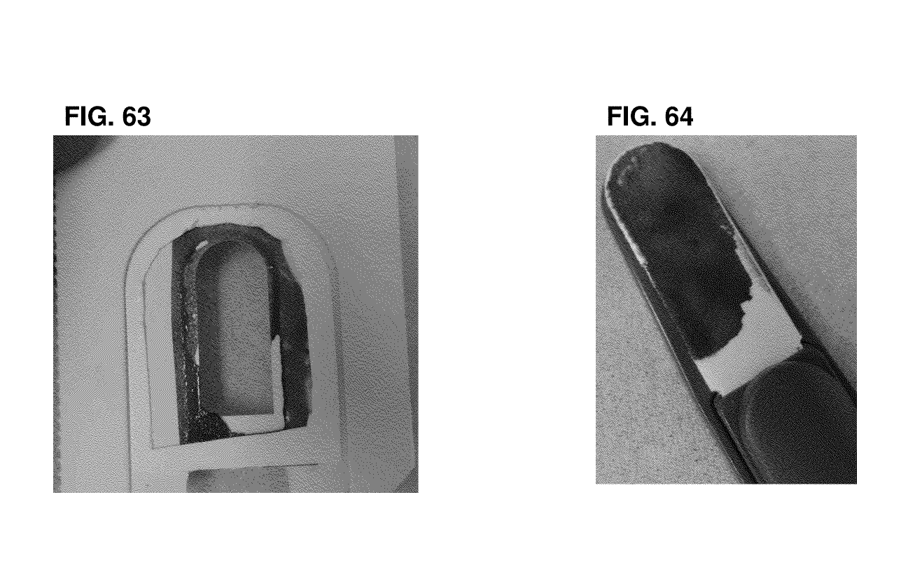

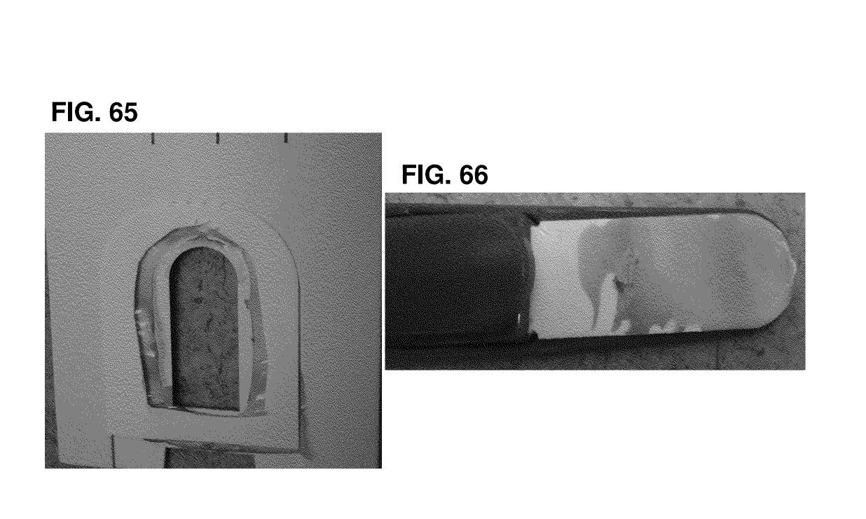

A portion of the film was cut large enough to cover the window of the Specimen Information Card (SIC) (FIG. 57). The film was affixed to the SIC card using an adhesive (FIG. 58-59). Using a specimen collector, a specimen sample was collected using 8 swipes. The SIC card was inverted and placed film side down on top of the specimen sample collected on the specimen collector. The SIC was secured with assistance from the slider (See FIG. 60). After 5 minutes, the SIC card was removed.

When the film was contacted with a specimen sample, the film was observed dissolving and percolating through the collection absorbent (FIGS. 61-62). As the preservative reagent is contained in the film, the dissolving and percolation transfers the preservative to the collection absorbent. About 1.2 mm punches were punched from the collection absorbent and analyzed (Table 13) using Identifiler Direct in conjunction with Prep-N-Go buffer to check for inhibition. The profiles of samples collected and contacted with the dissolvable film were compared to those not contacted with the dissolvable film.

TABLE 13
Criteria for analysis.

| Parameter | Criteria |
|---|---|
| Analytical Threshold | 75 RFUs |
| Stochastic Threshold | 200 RFUs |
| Intra locus balance | 60% |

Complete STR profiles were able to be obtained from samples which had CMC/PROTECT or PVP/PROTECT film components applied. Thus, the use of the dissolvable film did not completely inhibit the downstream amplification reaction typically used to analyzed collected specimens in forensic science.

### Example 7

### Evaluation of Dissolvable Film Characteristics

A desirable characteristic of a preservative film to be used with a specimen collector is the ability to dissolve and percolate through the collection absorbent. An analysis of the dissolvable stabilization film disclosed herein was conducted to assess dissolving and percolating characteristics.

A 5% solution of stabilization film solution was applied to a specimen collector sample and evaluated. Polyvinylpyrrolidone (PVP) was weighed and used to create a 5% PVP solution (5 grams per 100 mLs of liquid). A beaker was filled with 100 mLs of PROTECT solution (Table 12). The mixture was stirred to ensure all powder dissolved to the liquid form. After the powder dissolved, the beaker was sealed with parafilm. The beaker was inverted 10 times to ensure that none of the solution components settled or fell out of the solution. A small amount of the 5% stabilization film solution was poured onto a plastic bag that was placed inside a desiccant chamber. The liquid was allowed to dry, creating a film. A portion of the film was cut large enough to cover the window of the specimen collection card (SIC) (See FIG. 57). The film was affixed to the specimen collection card using an adhesive (See FIGS. 58-59).

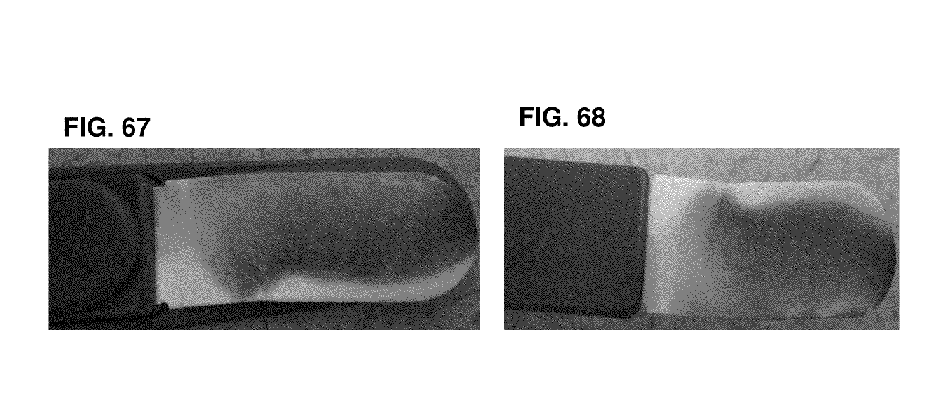

Using a specimen collector, a specimen sample was collected using 8 swipes. The SIC card was inverted and placed film side down on top of the specimen sample collected on the specimen collector. The SIC was secured with assistance from the slider (See FIG. 60). After 5 minutes, the SIC card was removed. The film was observed dissolving and percolating through the collection absorbent (FIGS. 61, 62, 64, 66-68). The amount of percolation appears to depend on the wetness of the collection absorbent and contact time between the film and the collection absorbent.

### Example 8

### Effectiveness of PROTECT Solution

In order for a preservative solution to be effective, it must completely or partially inhibit the activity of DNase, limit breakdown after exposure to high heat and humidity, and not inhibit a direct amplification reaction. The preservation effectiveness of the PROTECT solution was evaluated.

Seventy-two specimen collectors were cut using a fresh pair of clean scissors. Each collector absorbent was cut down the middle of the absorbent. Two Boston Bottles were filled with PROTECT solution (Table 12) and water (with food coloring added) and were labeled appropriately. A human subject was tasked to collect a sample specimen from the left cheek and one sample specimen from the right cheek using a specimen collector. Each human subject swiped a cheek 8 times with the specimen collector.

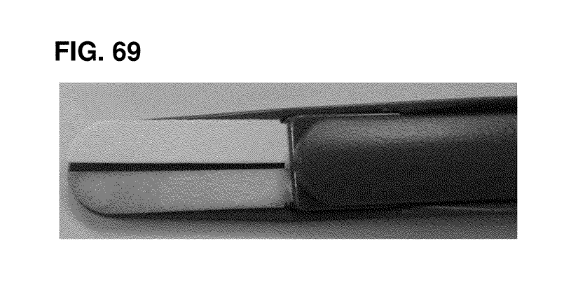

A pipette tip was used to separate the two halves of the collector absorbent. Then, three drops of the PROTECT solution or water was pipetted onto one half of the collector absorbent ensuring it did not transfer to the other half (FIG. 69). The collectors were placed in a transport pouch to dry for 2 hours. Half of the collectors were stored at the correct storage temperature in just the transport pouch (frozen, room temperature, 56° C., and 70° C.). Three laboratory Kimwipes® were soaked with 30 mLs of water and placed inside an individual sealable container. The remaining half of the collectors were placed at the correct storage temperature in the transport pouch, which was inside the sealable container with wet Kimwipes® (frozen, room temperature, 56° C., and 70° C.; each solution/temperature was in triplicate). The wet Kimwipes® were replaced with fresh wet Kimwipes® as needed to maintain a humid chamber. After the storage times, two 1.2 mm punches were removed from each half of the collector for direct amplificaiton using Identifiler® Direct. Also after storage, one 4.7 mm punch was removed from each half of the collector for extraction and quantification. The criteria used for analysis is provided in Table 13. The Identifiler® Direct profiles between the use of the PROTECT solution and not using the PROTECT solution were compared.

Over an extended period of time, the PROTECT solution used herein limits DNA degradation, while not inhibiting downstream amplification analysis. After storage at 56° C. at relatively low humidity (<10%) for twenty-seven weeks, 4.7 mm punches were taken from split collectors that had been treated with the PROTECT solution or left untreated. After twenty-seven weeks, the untreated collectors had an average degradation index value of 12.99, while the PROTECT solution treated collectors had an average degradation index value of 1.73. This difference shows that the untreated samples displayed a much higher level of degradation compared to the treated samples.

### Example 9

### Dissolvable Preservative Film on a Liner

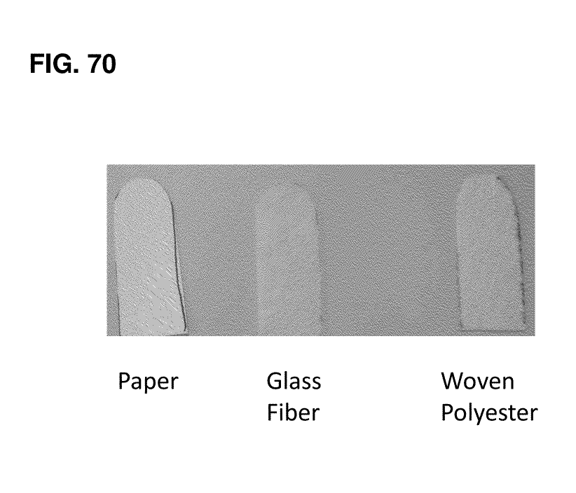

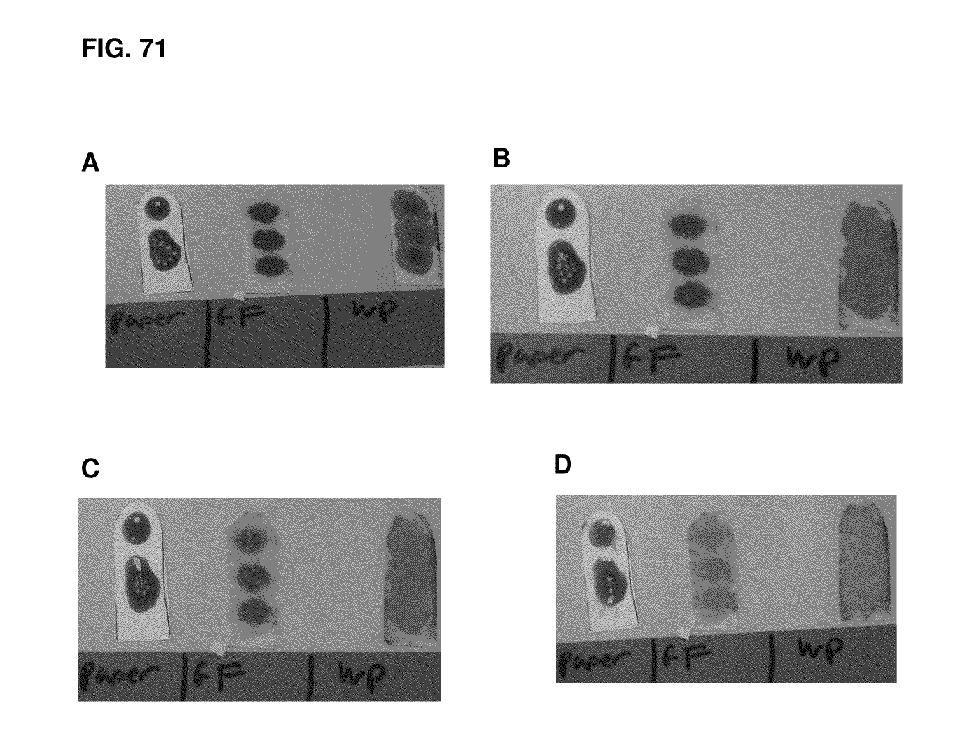

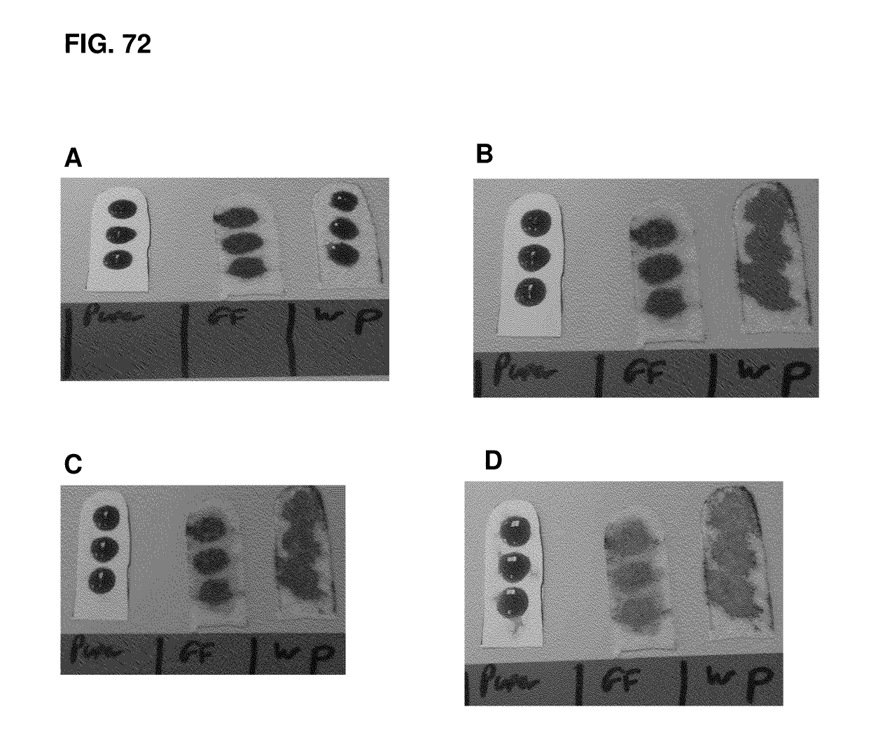

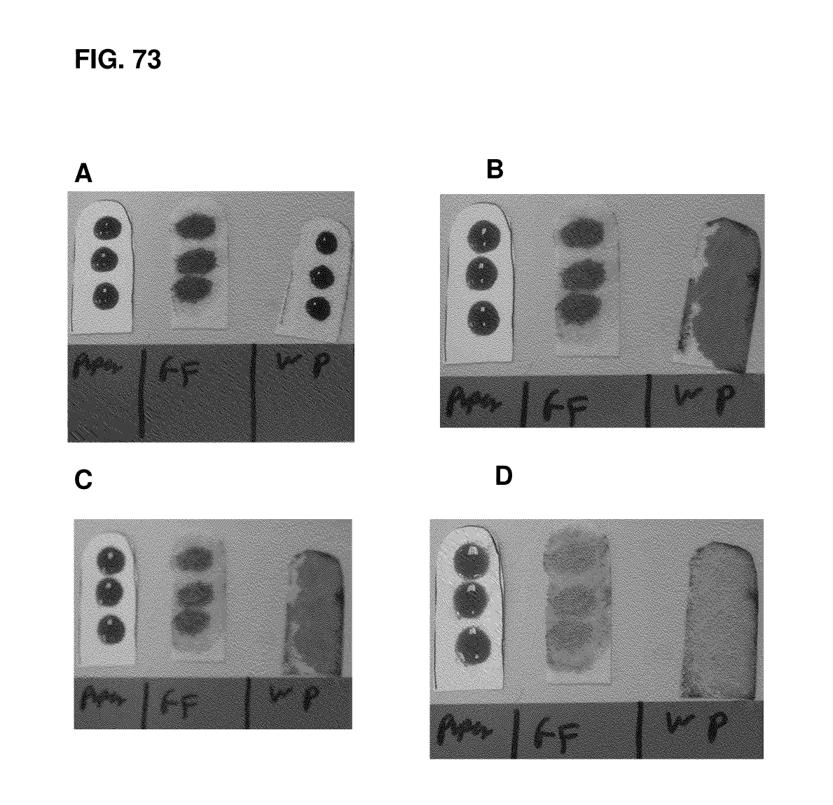

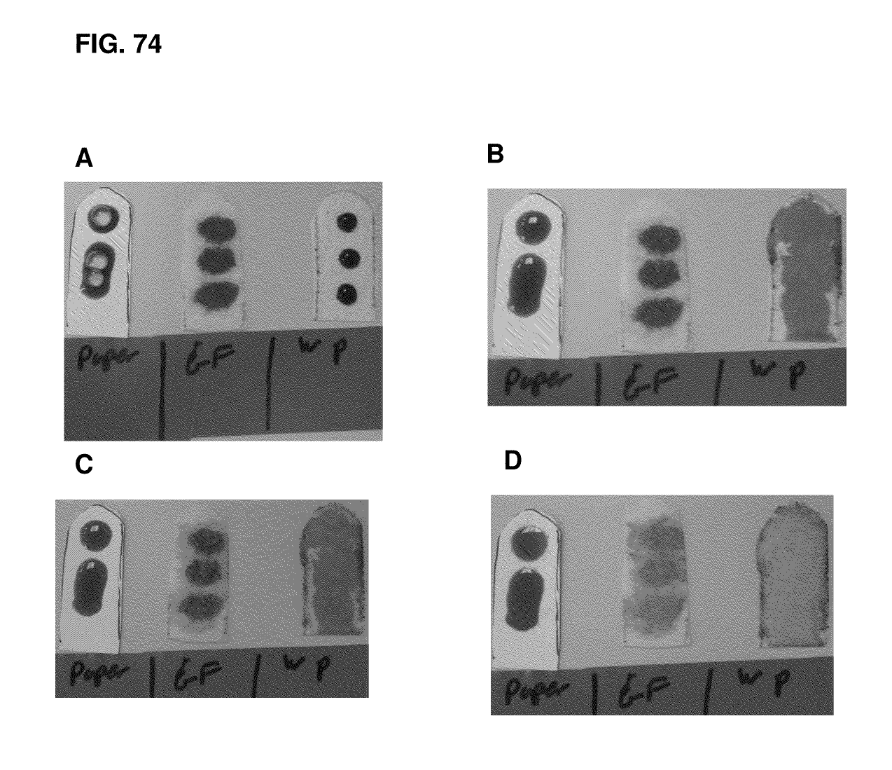

This experiment was designed to test the transfer of PROTECT solution that had been applied to a liner before drying and transferring to a Buccal DNA Collector™ sample. The following set of materials was evaluated: glass fiber; woven polyester; and a no tear paper with liner. Materials were cut into the shape of the paper in a Buccal DNA Collector™ (FIG. 70). The solutions evaluated were 5% PVP PROTECT (40K and 10K), and 0.25% or 0.5% CMC PROTECT. 100 μl of each solution was pipetted (3 pipette tips 33.3 μl each) onto each material. It was observed that paper does not allow the liquid to absorb and percolate throughout the paper. Instead, the liquid remains on the surface and dries. Glass fiber readily absorbs although percolation is slow and film component dependent. Woven polyester initially bubbles but once it is absorbed it begins to percolate (FIGS. 71-74). The solution was allowed to dry on or in the liner.

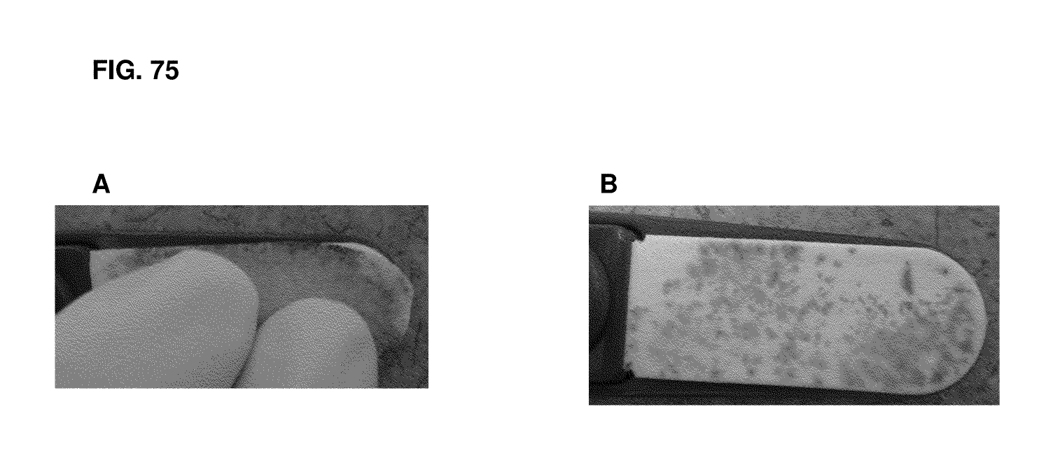

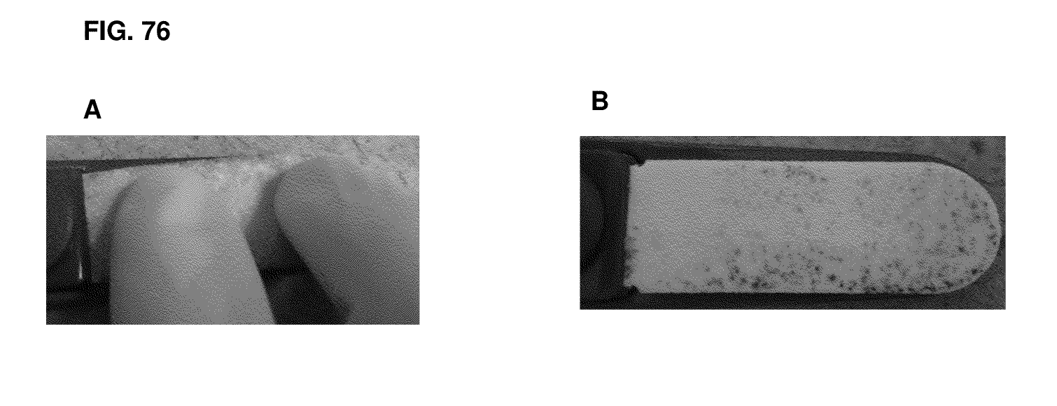

Using a specimen collector, a specimen sample was collected from a subject by having the subject first rub the tongue against the cheek in an attempt to increase wetness, followed by swiping the sample collector 8 times against the cheek. PVP 10K and CMC 0.25% on woven polyester were placed on top of the paper and held with moderate pressure for 5 minutes to transfer (FIGS. 75 and 76). As is shown in the figures, transfer occurs where the collector paper is wet. Additionally, PVP 10K appears to more easily transfer out of woven polyester as compared to CMC 0.25%.

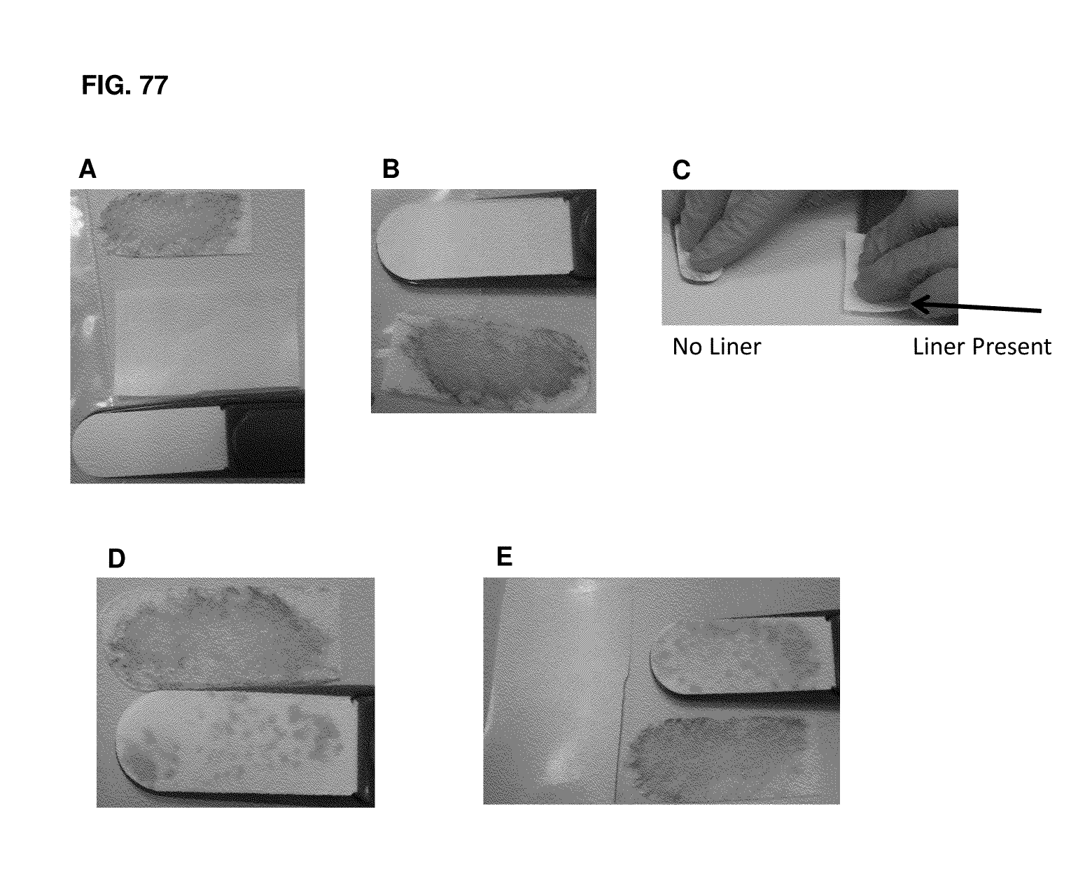

Use of a liner during transfer of PVP 10K PROTECT solution from woven polyester was evaluated. In short, PVP 10K PROTECT on woven polyester was placed on top of a sample collector paper having a buccal sample thereon and held with moderate pressure for 5 minutes using a liner, or without using a liner (FIG. 77). As seen in the figure, it appears that use of a liner may increase transfer of solution.

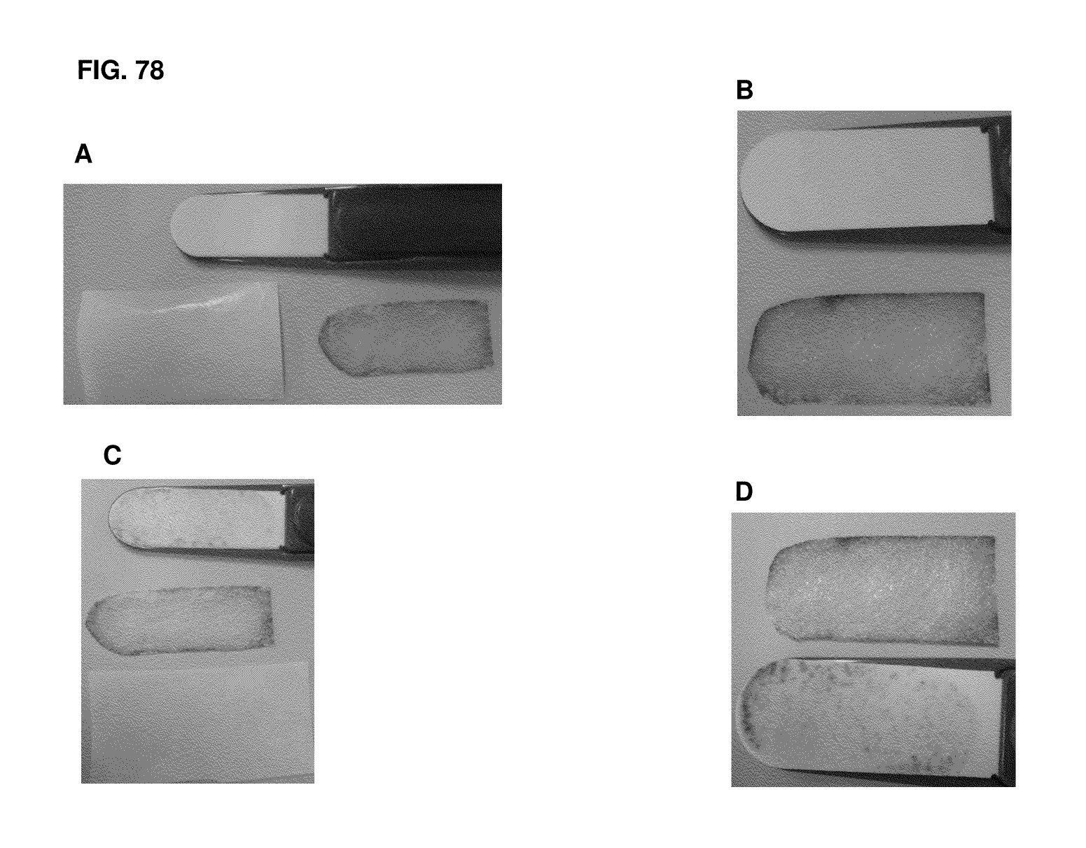

Use of a liner during transfer of CMC 0.25% PROTECT solution from woven polyester was also evaluated. CMC 0.25% on woven polyester was placed on top of a sample collector paper having a buccal sample thereon and held with moderate pressure for 5 minutes using a liner, or without using a liner (FIG. 78). As seen in the figure, it appears that the amount of transfer is similar between the use of a liner and no liner use.

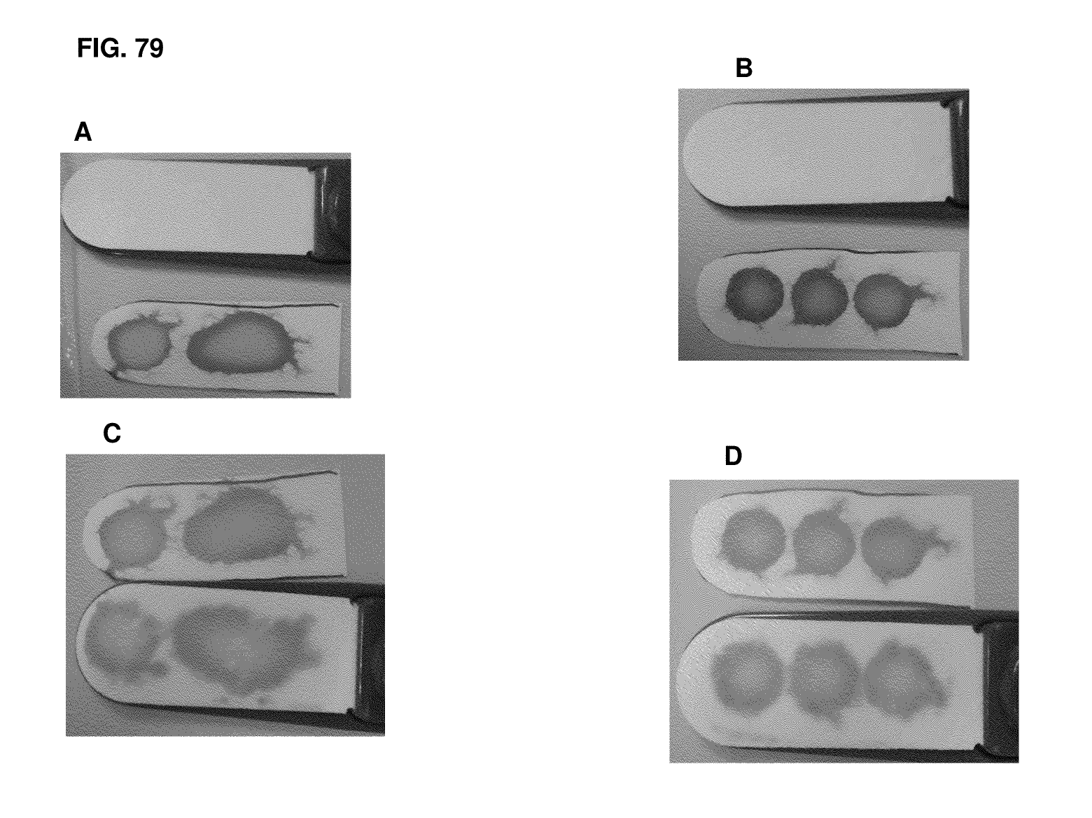

Next, transfer of 5% PVP 40K PROTECT or 5% PVP 10K PROTECT were compared (FIG. 79). No liner was used in this experiment during transfer. To test the transfer of solution, the paper of a sample collector was first moistened with 50 μL, and PVP 40K PROTECT or PVP 10K PROTECT on paper were placed on top of sample collector paper and held with moderate pressure for 5 minutes. As seen in the figure, it appears that PVP 40K PROTECT and PVP 10K PROTECT on paper transfer easily in the exact spot where the solution was located.

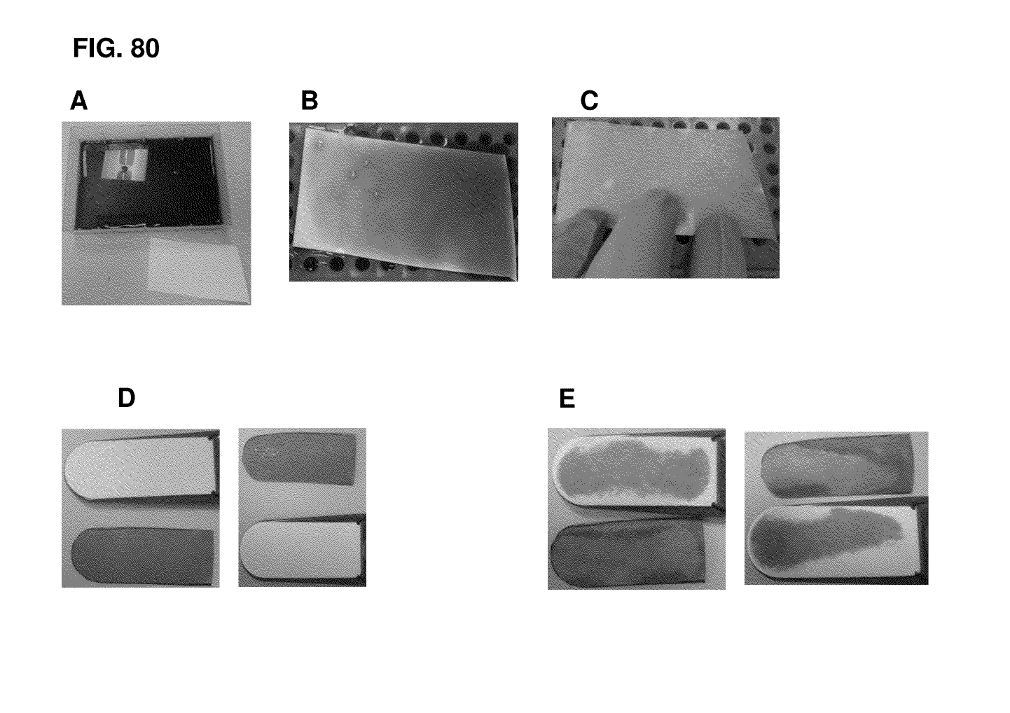

This experiment was designed to evaluate the transfer of PROTECT solution that had been evenly applied to paper. In short, a dissolvable film was cast by submerging paper in 40K PVP PROTECT solution (FIGS. 80A, B). After drying (FIG. 80C), the paper/film was cut into the shape of the paper in a Buccal DNA Collector™ (FIG. 80D) and placed on top of a sample collector paper that had been moistened with 50 μL water, and held with moderate pressure for 5 minutes. As seen in FIG. 80E, 40K PVP PROTECT solution on paper transfers easily when the sample collector is wet.

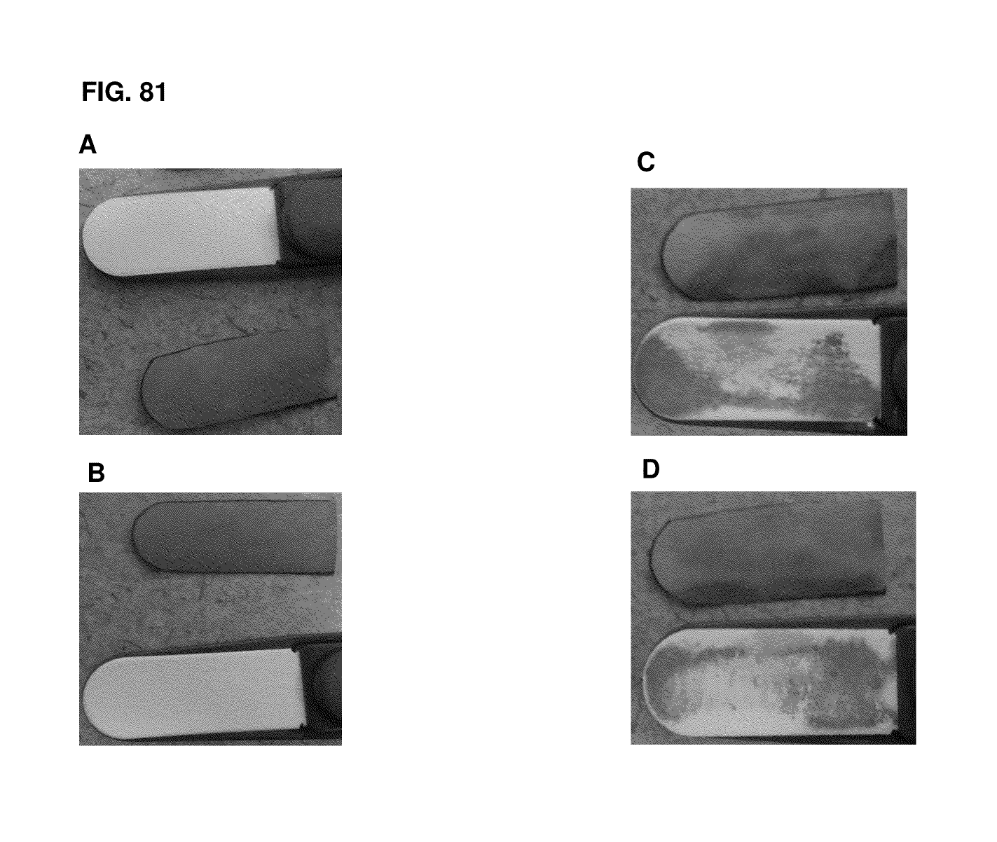

The PROTECT solution evenly applied to paper was further evaluated using Buccal DNA Collectors™ that had been used for collecting samples from a subject. Specimen samples were collected from a subject as described above wherein the tongue was used to moisten the check before sample collection. As seen in FIG. 81, 40K PVP PROTECT solution transfers easily when the sample collector is wet. As such, transfer and percolation of solution are dependent on wetness of the sample. Additionally, it appears that a subject rubbing the tongue on the cheek before sample collection increases wetness of the sample collector, thereby enhancing transfer and percolation of solution.

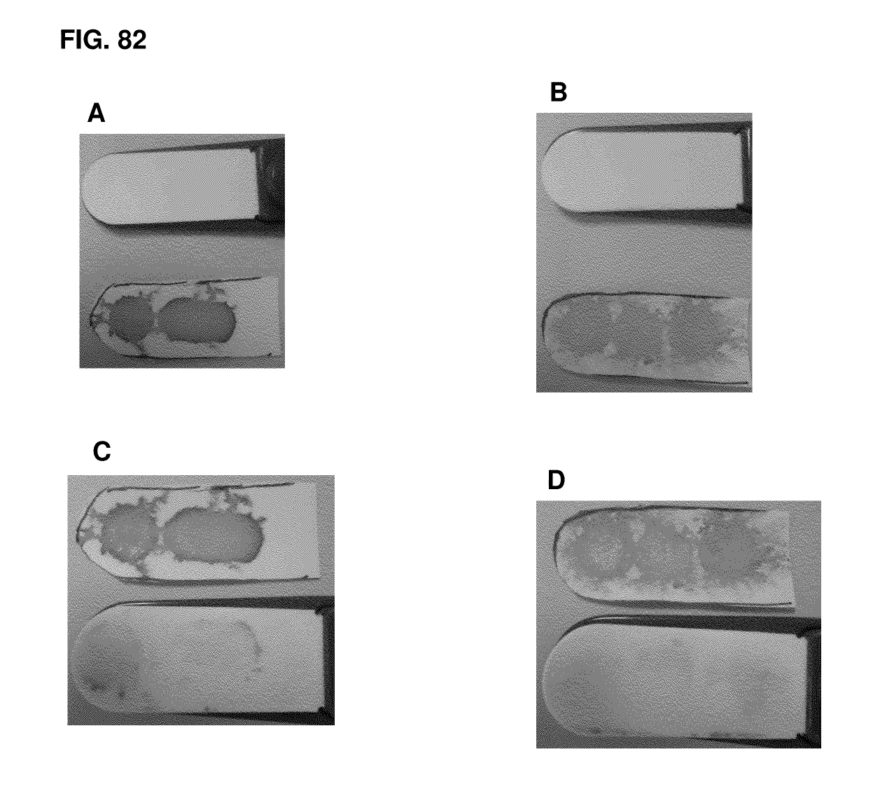

In this study, transfer of PROTECT solution from 0.25% CMC PROTECT or 0.5% CMC PROTECT was compared (FIG. 82). No liner was used in this experiment during transfer. The transfer of solution was evaluated as described above. As seen in the figure, it appears that although PROTECT solution was transferred to the wet sample paper, transfer appeared to be less efficient when compared to PVP solutions as evaluated in this example above.

These results appear to show that no tear paper, when combined with PVP, appears to be an efficient liner for transfer.

### Example 10

### Dissolvable Preservative Film on Various Liner Materials

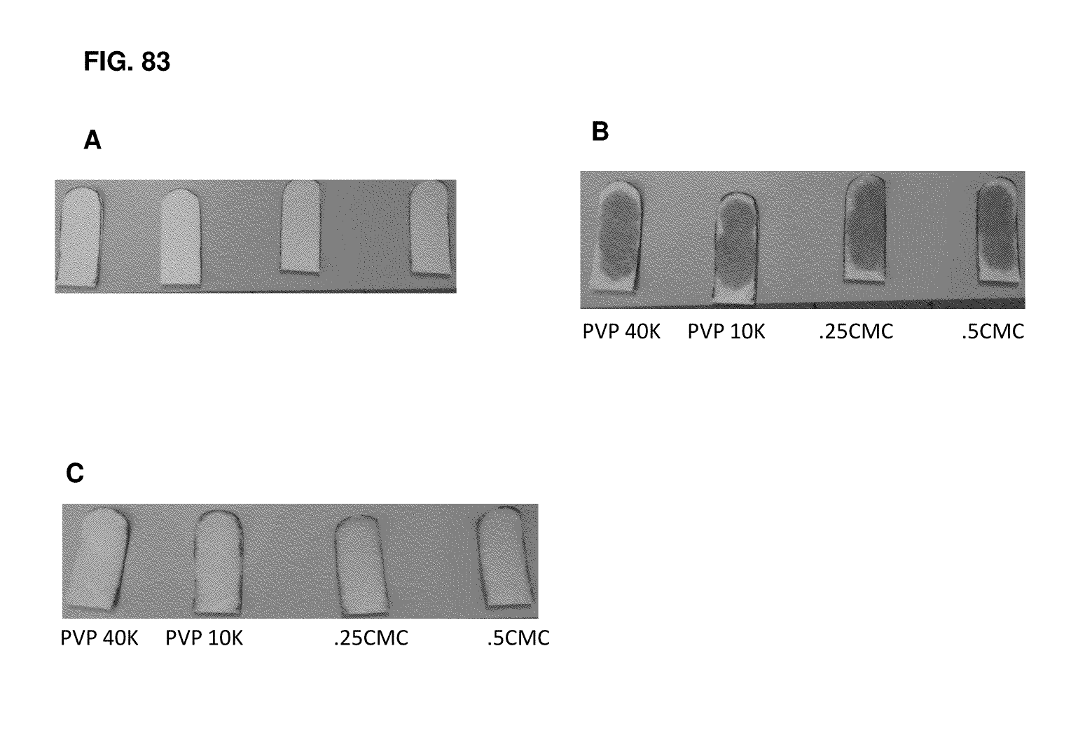

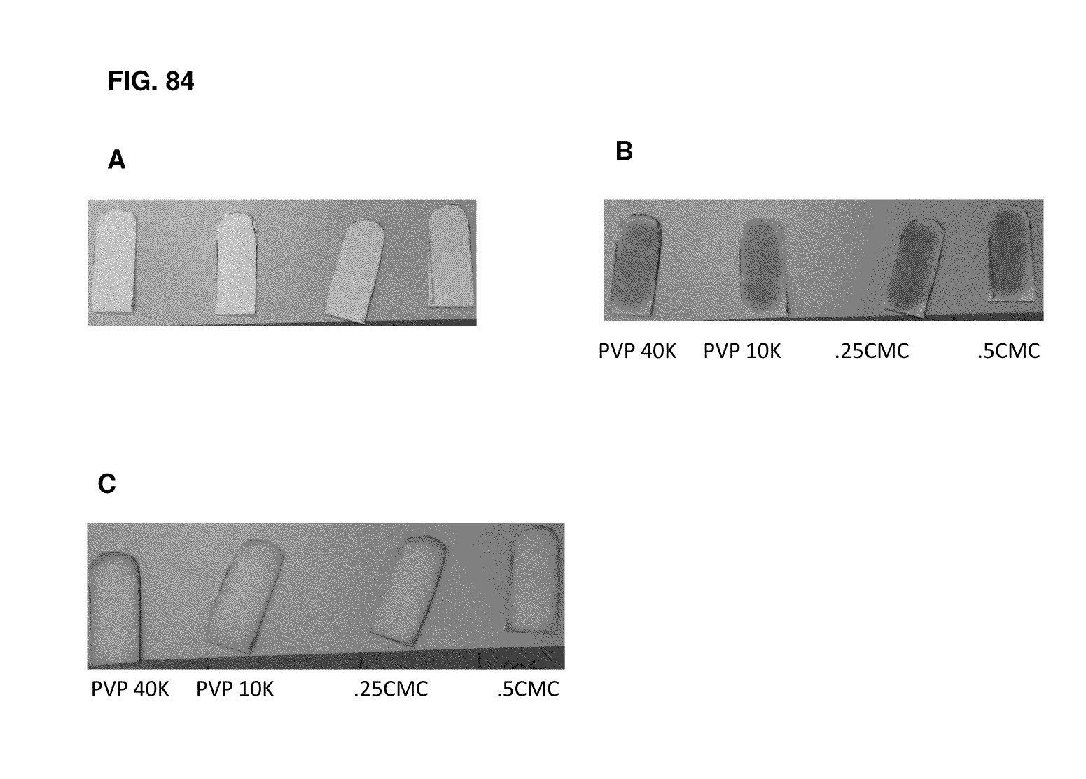

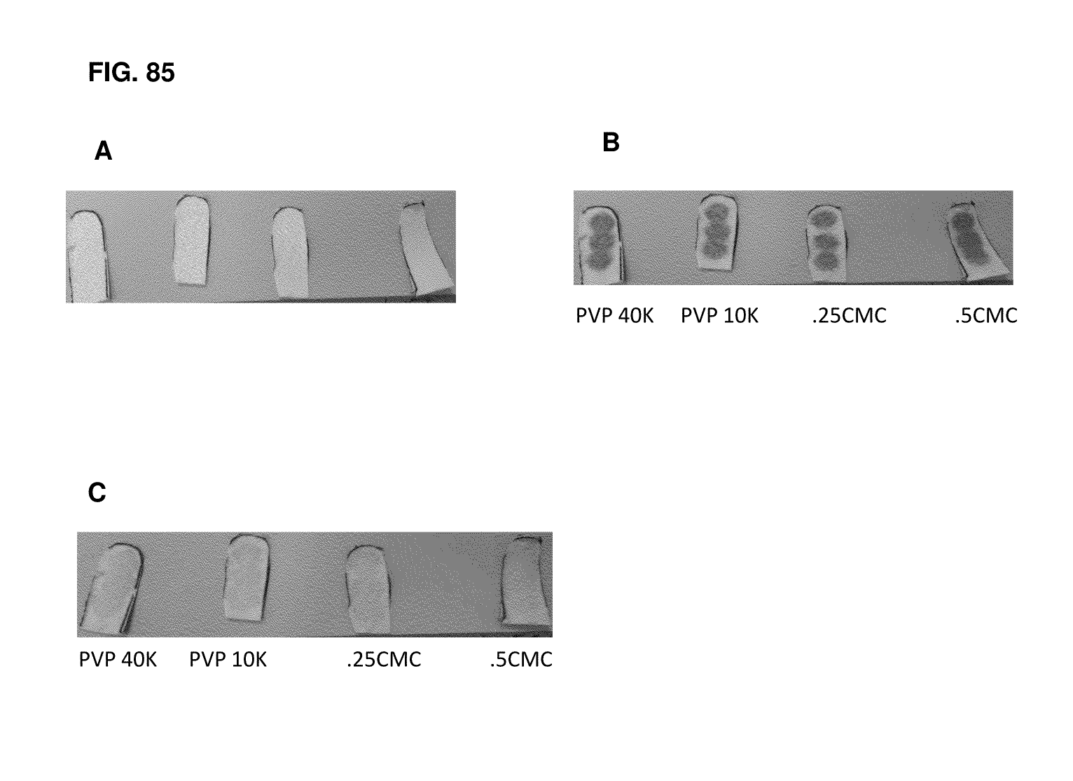

This experiment was designed to evaluate the transfer of PROTECT solution that had been applied to paper obtained from I.W. Tremont before drying and transferring to a Buccal DNA Collector™ sample. The following set of materials was evaluated: RG paper which is a glass product with acrylic binder laminated with nonwoven high strength spun bonded polyester; LL72 paper which is a glass product laminated on both sides of the glass material with nonwoven high strength spun bonded polyester; and B-85 paper which is a binderless borosilicate glass fiber product. Materials were cut (4 each) into the shape of the paper in a Buccal DNA Collector™ (FIGS. 83-85). The solutions evaluated were as described in Example 10. First, 100 μl of each solution was pipetted (3 pipette tips 33.3 μl each) onto each material. It was observed that RG and LL72 readily absorb and solution readily percolates throughout the materials (FIGS. 83, 84). It was observed that B-85 paper readily absorbs the solution, but the solution percolates very slowly (FIG. 85). The solution was allowed to dry on or in the liner.

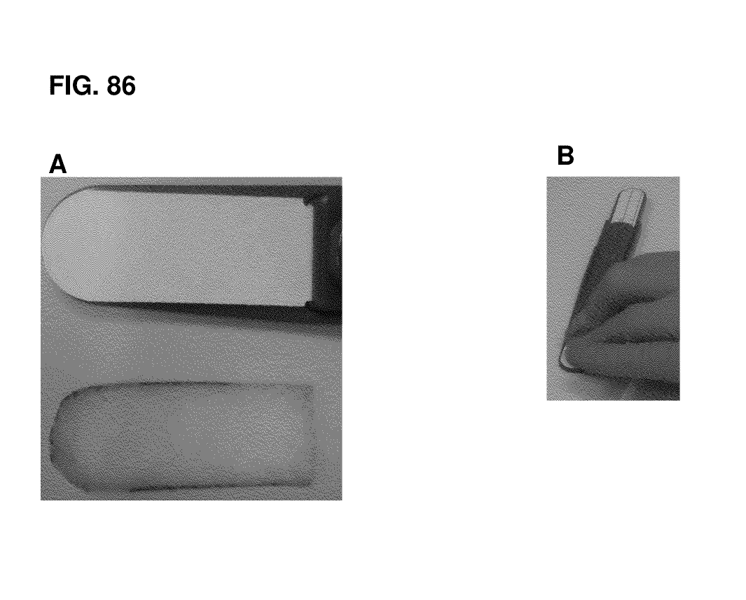

To test the transfer of solution from the various liners, the paper of Buccal DNA Collectors™ was first moistened with 50 μL water was first added to the paper. Liners were then placed on top of the paper and held with moderate pressure for 5 minutes (FIG. 86). No transfer occurred when the LL72 paper was tested. The causes for the failed transfer were not immediately apparent and may be determined upon further investigation. For instance, the solution may have percolated through the glass material of the paper and onto the back of the lining, thereby preventing the contact of the solution with the sample collector paper.

### Example 11

### Effectiveness of Dissolvable Preservative Film on Paper Liner

The ability to dissolve and percolate through the collection absorbent and the preservation effectiveness of the preservative film material of PROTECT solution on a paper liner described in Examples 4 and 5 and subsequently applied to a Buccal DNA Collector™ sample was evaluated.

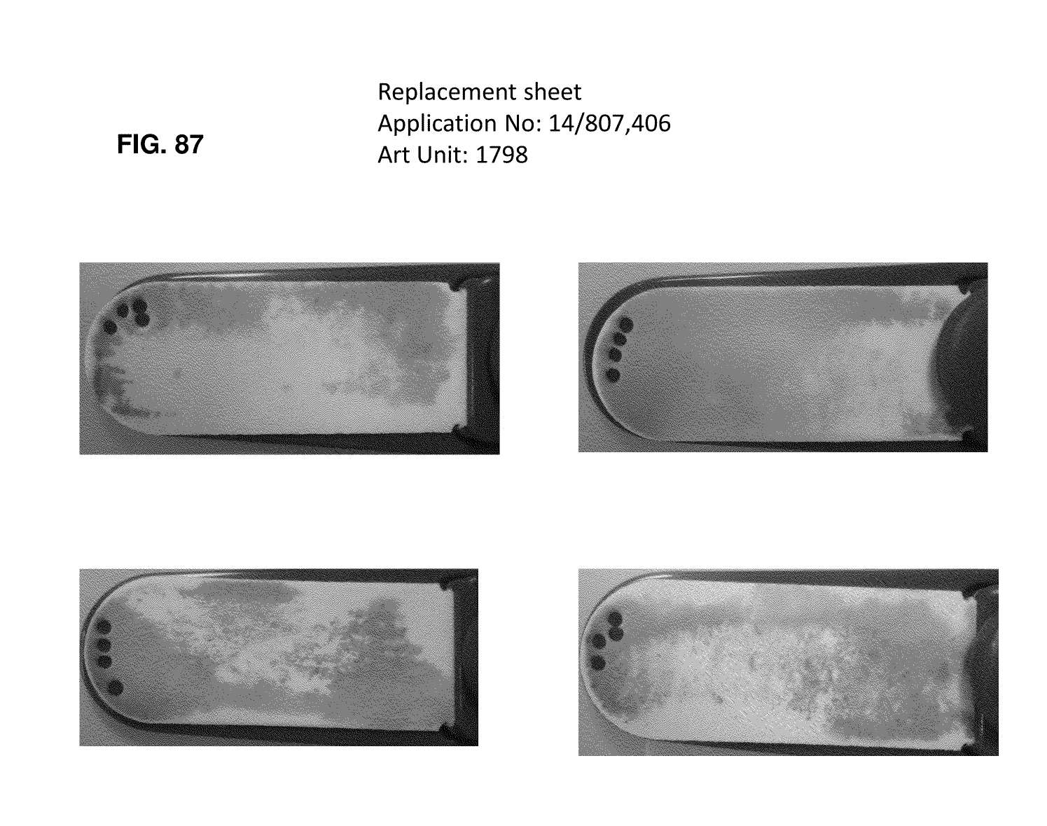

Eight specimens were collected from 2 individuals using Buccal DNA Collectors™, and a 5% PVP PROTECT solution was transferred to the BDCs via the paper matrix as described in the Examples above. It appears that collectors were not evenly wetted during sample collection, given that not all areas of the sample collectors turned blue from the preservative film (FIG. 87). Given that the preservative solution is blue, the colored portions of the collector indicate where transfer had occurred. Six (n=6) out of the eight (n=8) samples tested were used for further analysis.

Two 1.2 mm punches were taken from each collector for direct amplification. The punches were taken from the blue areas of the collectors (FIG. 87) where preservative solution transfer had occurred. The punch samples were amplified using the PowerPlex® Fusion system to determine the level of inhibition. In short, 10 μl of PunchSolution™ buffer of the PowerPlex® system was added to each to each punch sample and incubated on a heat block for 30 minutes at 70° C. or until dry. PowerPlex® Fusion reaction mix (25 ul) was then added and samples were amplified with a thermal cycler using 25 cycles. The resulting fragments were then separated on a 3130 Genetic Analyzer, and the data was analyzed using GeneMapper® v 3.2.1 software.

Overall, inhibition issues were not observed in any samples. The overall interlocus balance is not very high given that these are fresh collectors; however, it is consistent with the results obtained previously from untreated samples. The PowerPlex® Fusion system encompasses 24 loci over a large range of fragment sizes. Due to this large range and number of loci, the interlocus balance may be less than what is seen with 16 loci amplification kits.

As such, this study showed that a preservative can be applied to a paper liner and then transferred to a Buccal DNA Collector™. The collector can be subsequently punched, lysed with PunchSolution™, and amplified using PowerPlex® Fusion. Further studies directed at determining PROTECT solution transfer, rates of inhibition with various transfer conditions, and sample preservation levels may be evaluated further.

### Example 12

### Dissolvable Preservative Film on Raeton™ Paper Liner Materials

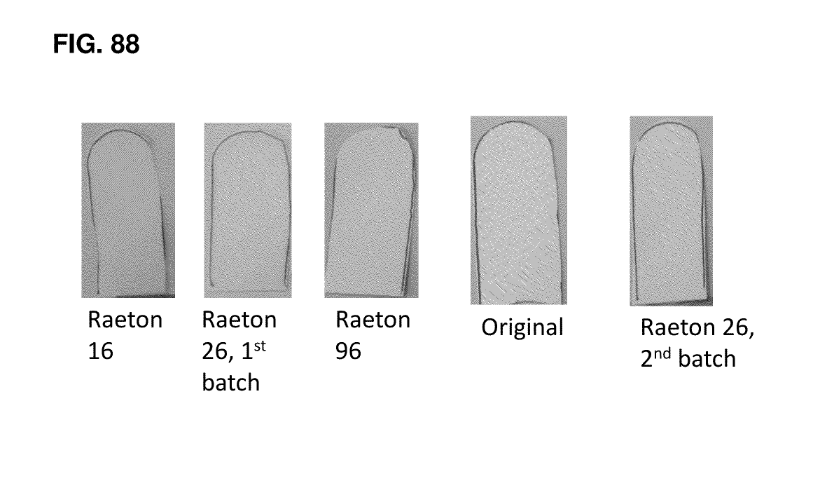

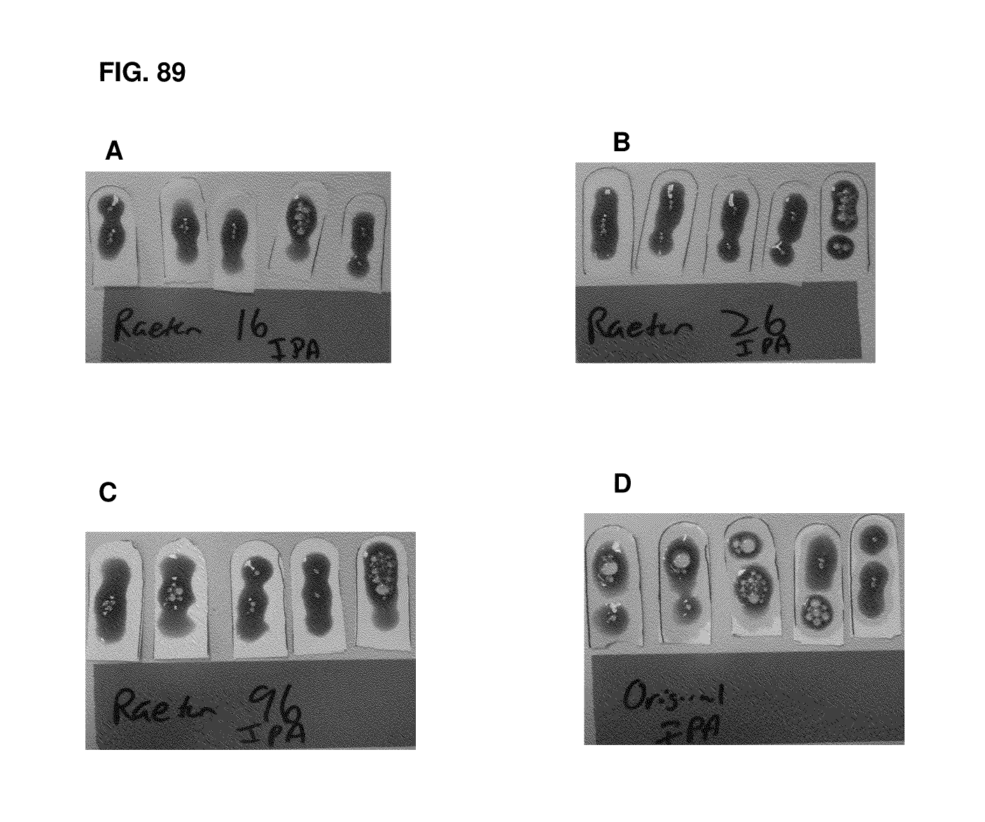

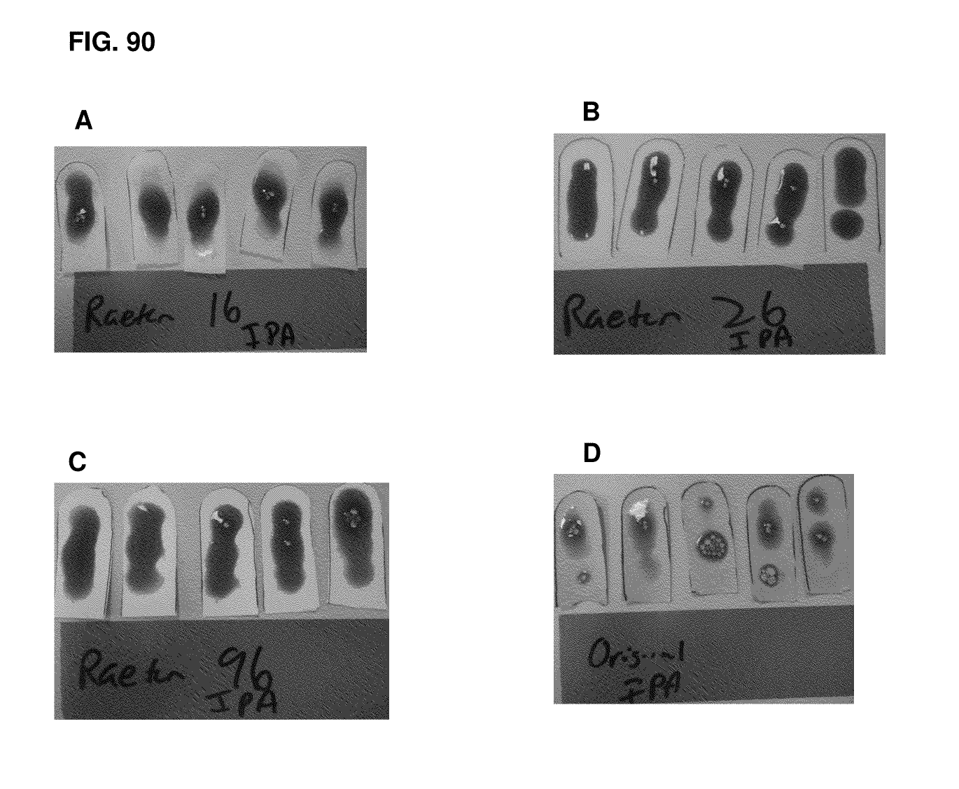

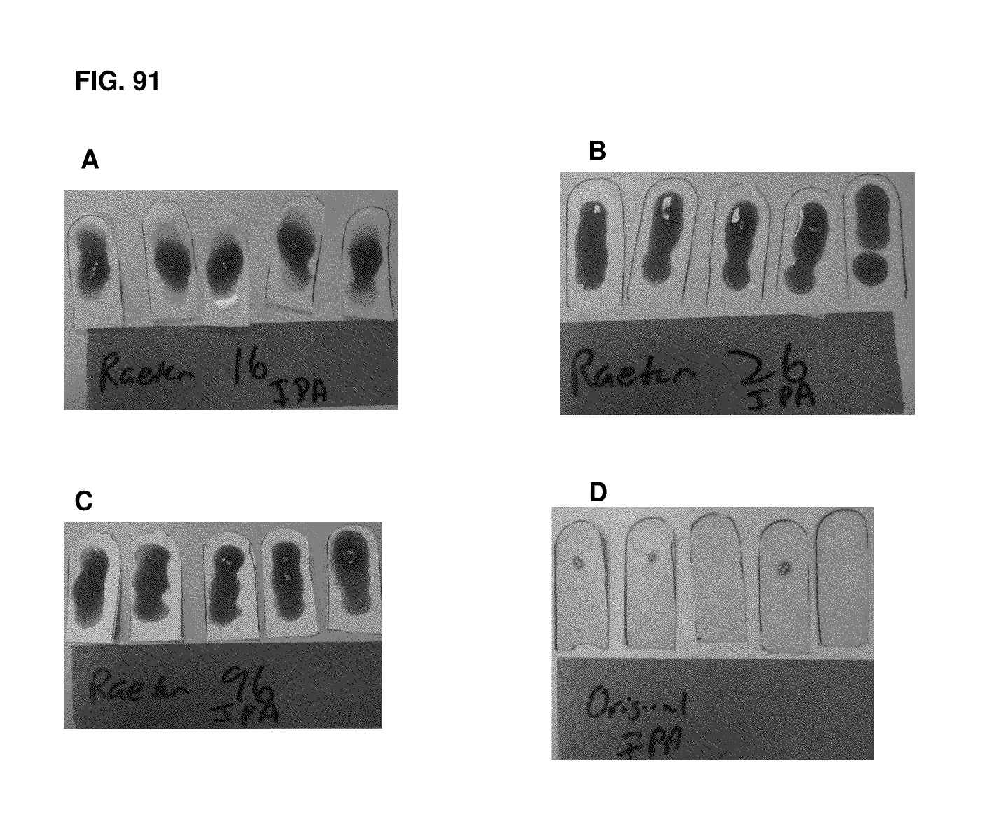

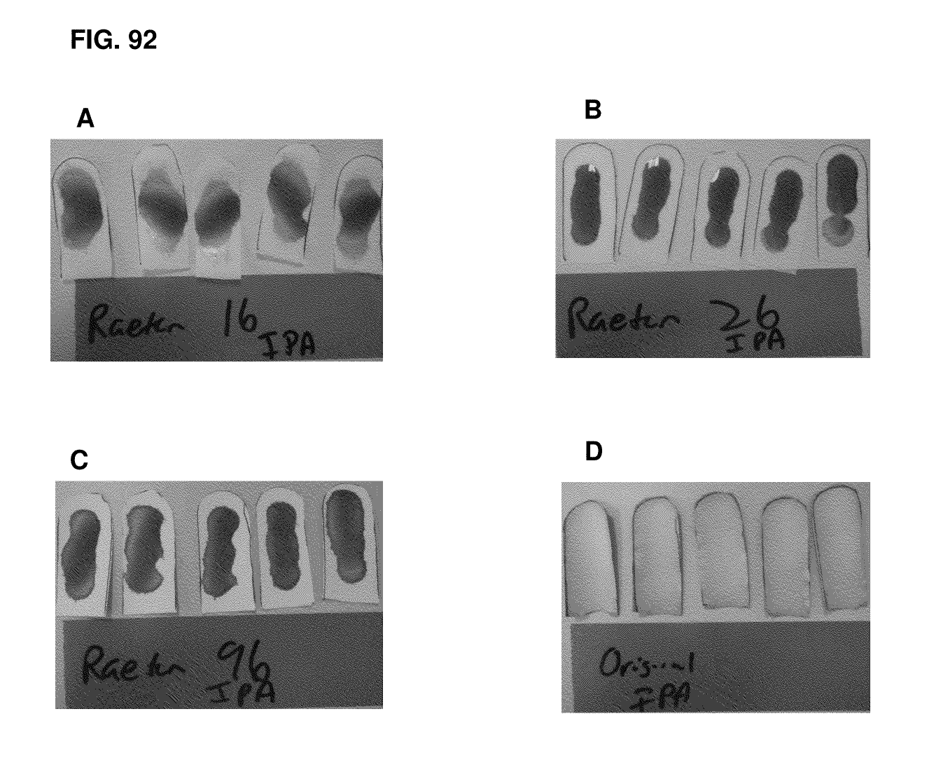

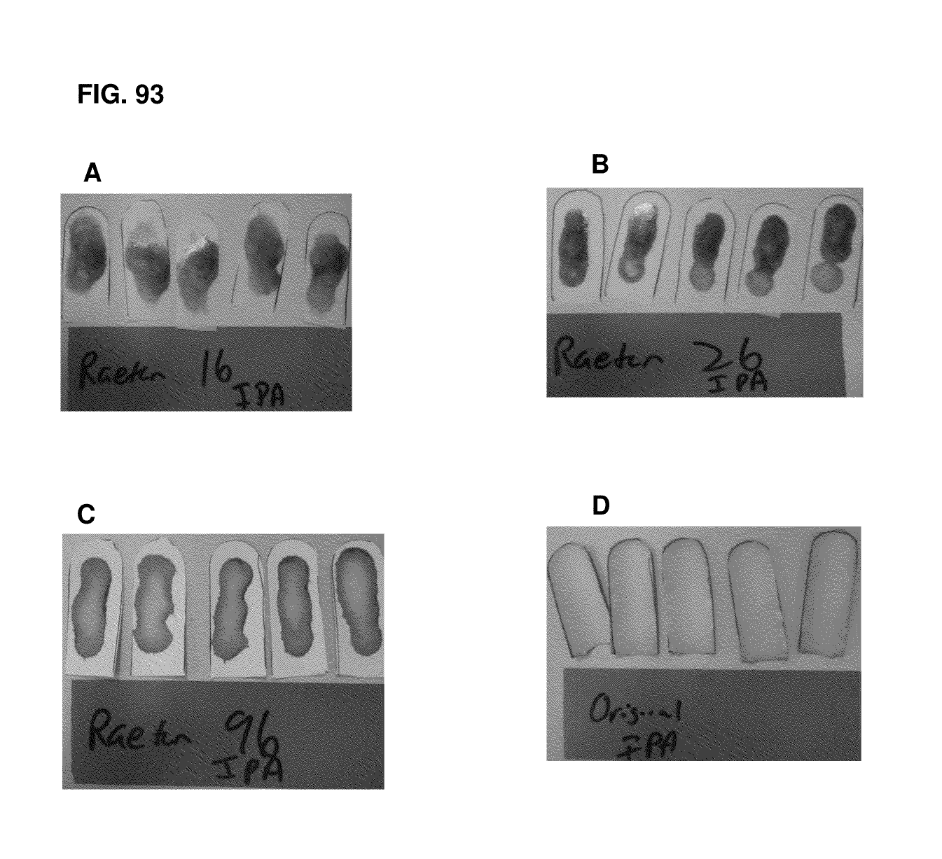

This experiment was designed to evaluate the transfer of PROTECT solution that had been applied to Raeton™ paper obtained from Griff Applied Laminates before drying and transferring to a Buccal DNA Collector™. RAETON™ paper products typically comprise paper/film/paper laminated products. The following set of materials was evaluated: RAETON™ 16; RAETON™ 26 from two different batches; RAETON™ 96; and a different batch of RAETON™ 26. Original paper was also used for comparison. Materials were cut into the shape of the paper in a Buccal DNA Collector™ (FIG. 88). The solution evaluated was 5% PVP 40K PROTECT comprising 20% isopropyl alcohol (IPA). 100 μl of each solution was pipetted (3 pipette tips 33.3 μl each) onto each material, and allowed to dry (FIGS. 89-93).

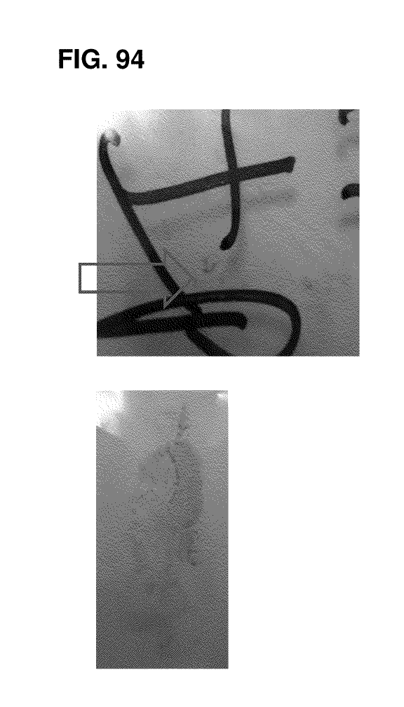

It was observed that solutions applied to RAETON™ 16 and 26 can remain tacky and stick to a packaging surface such as a plastic bag (FIG. 94). Solutions applied to materials with textures more similar to paper (RAETON™ 96 and original paper) remain dry and did not stick to a packaging surface.

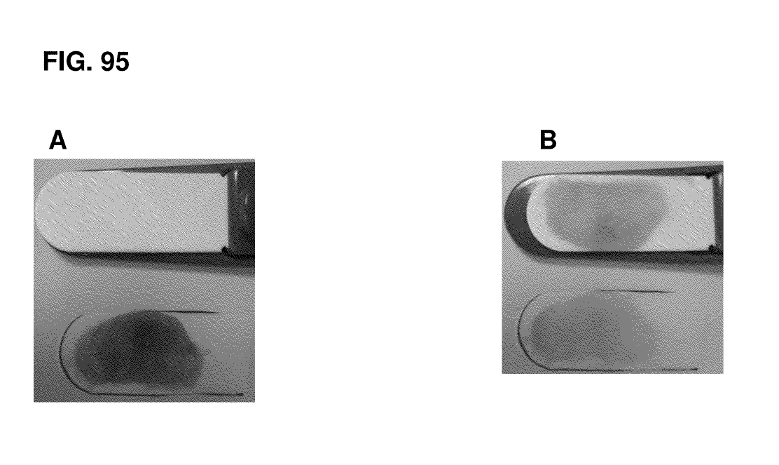

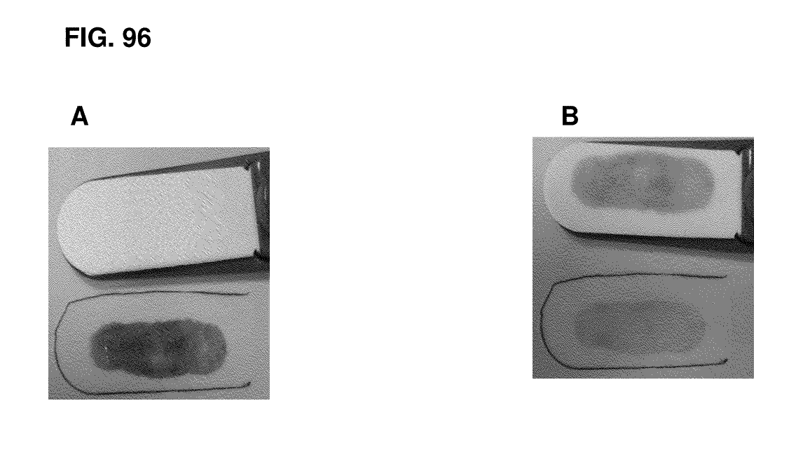

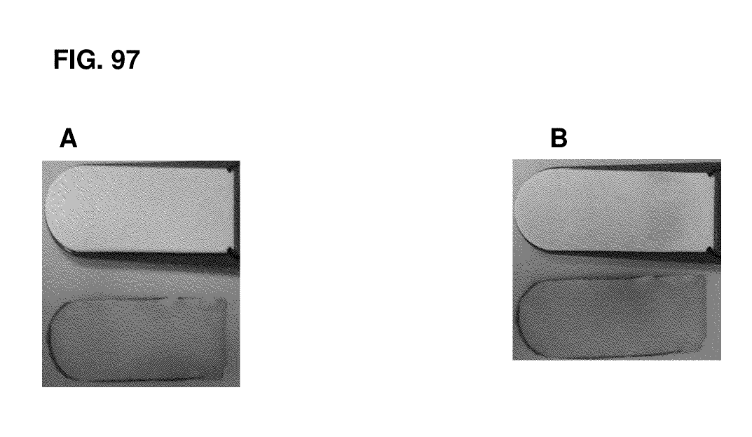

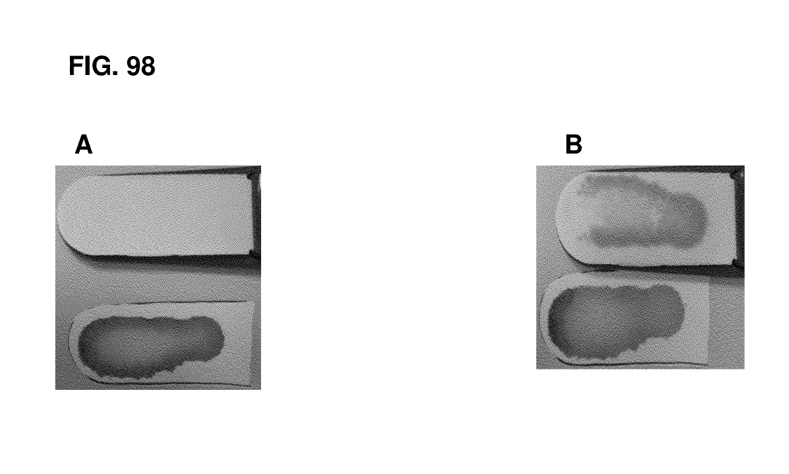

To test the transfer of solution from the various liners, 50 μL water (3 pipette tips 16.66 μl each) was first added to the paper of a sample collector. The various RAETON™ papers and the original paper having PROTECT+IPA film thereon were then placed on top of the paper and held with moderate pressure for 5 minutes. Transfer of PROTECT+IPA material from RAETON™ paper was efficient, whereas transfer from original paper was reduced (FIGS. 95-98). The causes for the failed transfer from original paper were not immediately apparent and may be determined upon further investigation. For instance, the solution may have percolated through the paper material, thereby preventing the contact of the solution with the sample collector paper.

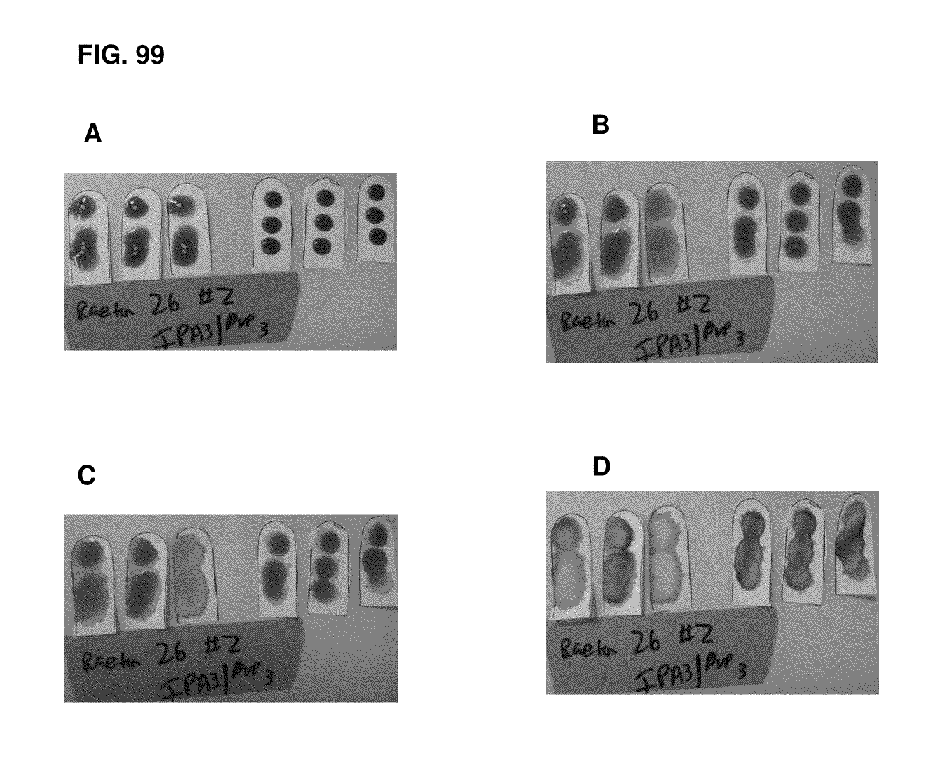

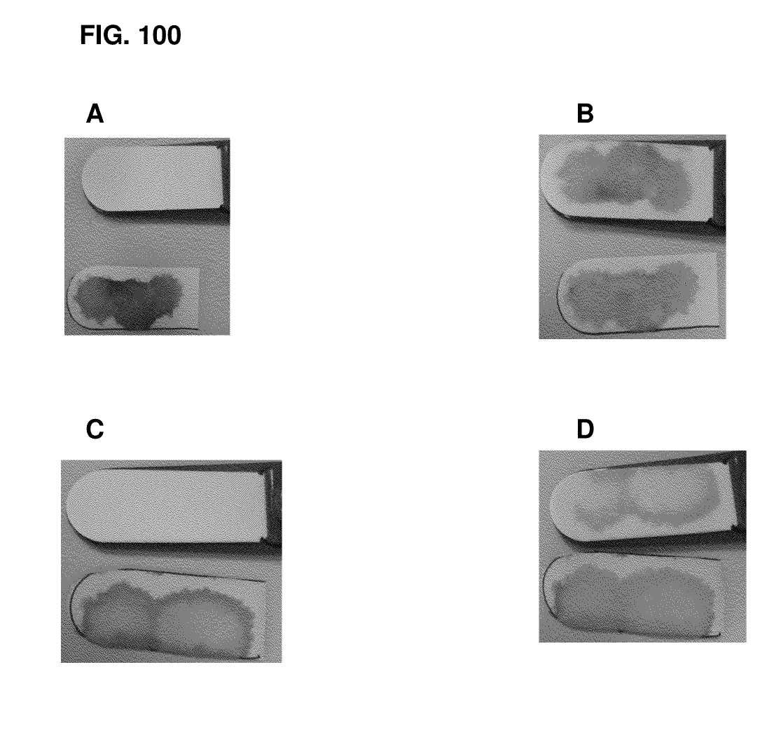

The addition of IPA to the PROTECT solution was also evaluated by comparing film formation using 5% PVP (40K) PROTECT with or without IPA on one of the batches of RAETON™ 26 paper. Film formation using the solutions was as described in this example above (FIG. 99). It was observed that PROTECT solution does not bead as much on RAETON™ 26 paper when using IPA compared to solution without IPA. When the original paper is used, percolation readily occurs. Transfer of 5% PVP (40K) PROTECT with or without IPA on RAETON™ 26 paper was also evaluated. No transfer occurred when the LL72 paper was tested (FIG. 100). Both materials easily transferred to the wet collection paper.

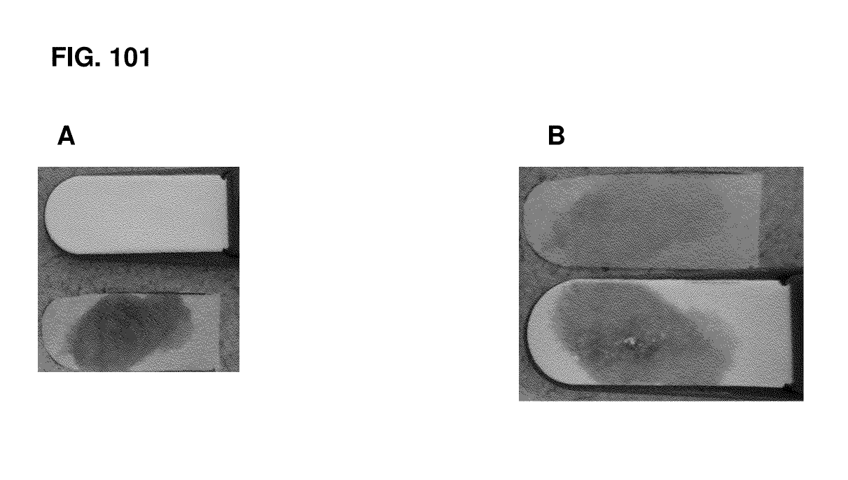

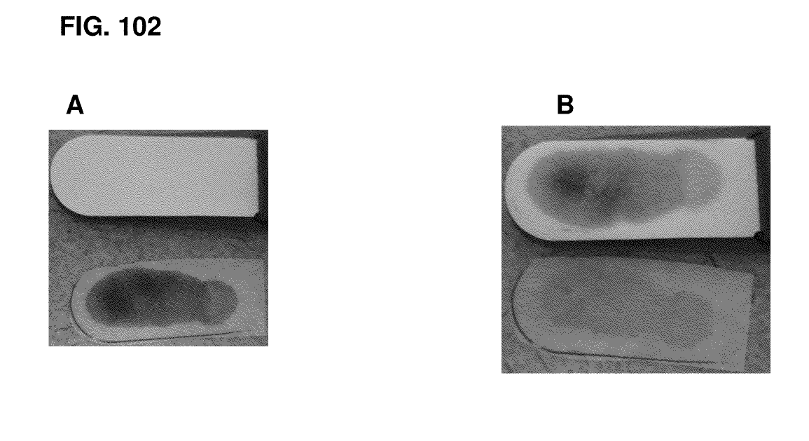

Transfer of solution comprising IPA was also evaluated from RAETON™ 16 and 26 paper to Buccal DNA Collector™ that had been used for collecting samples from a subject. Sample collection was as described above, wherein the tongue was rubbed against the cheek of the subject in an attempt to increase wetness. RAETON™ 16 and 26 paper having PROTECT+IPA film thereon were then placed on top of the paper and held with moderate pressure for 5 minutes. Similar to the control studies with water, transfer and percolation were successful and dependent on wetness of the sample collector (FIGS. 101-102).

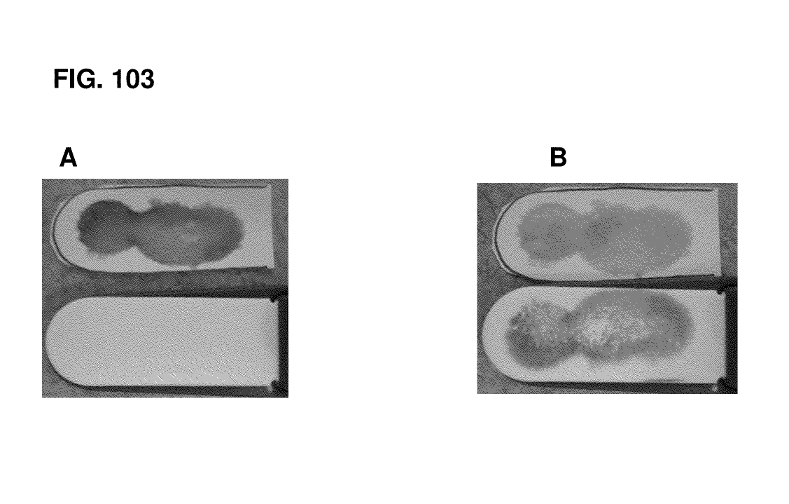

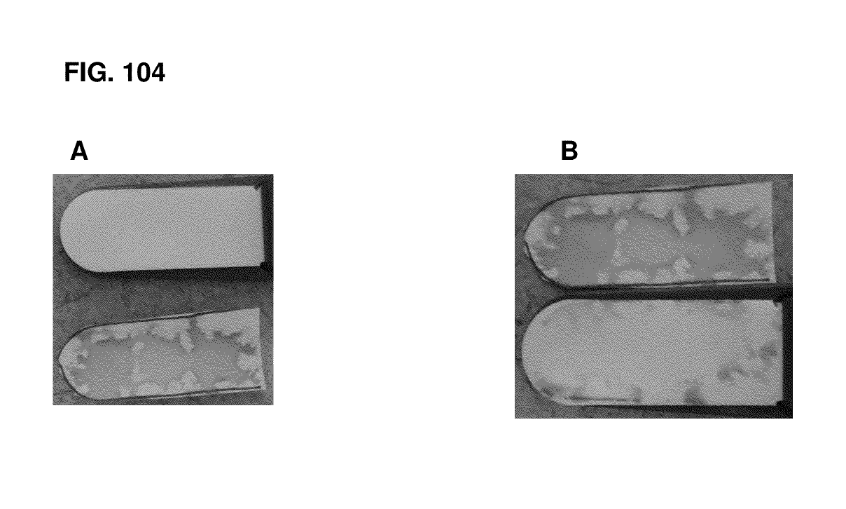

Similarly, transfer of solution without IPA was also evaluated from RAETON™ 26 and original paper to Buccal DNA Collector™ that had been used for collecting samples from a subject. RAETON™ 16 and original paper having PROTECT without IPA thereon were then placed on top of the paper and held with moderate pressure for 5 minutes. RAETON™ 26 transferred more easily than the original paper. (FIGS. 103-104).

### Example 13

### Evaluation of Method of Applying Film Solution onto Paper Liner

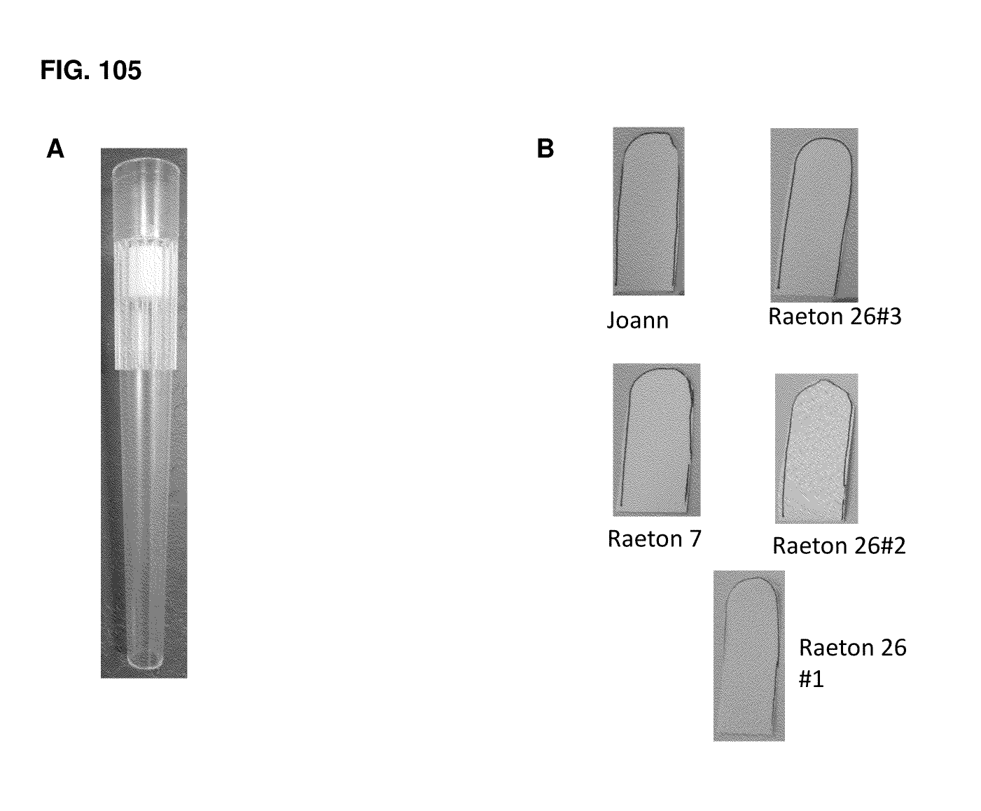

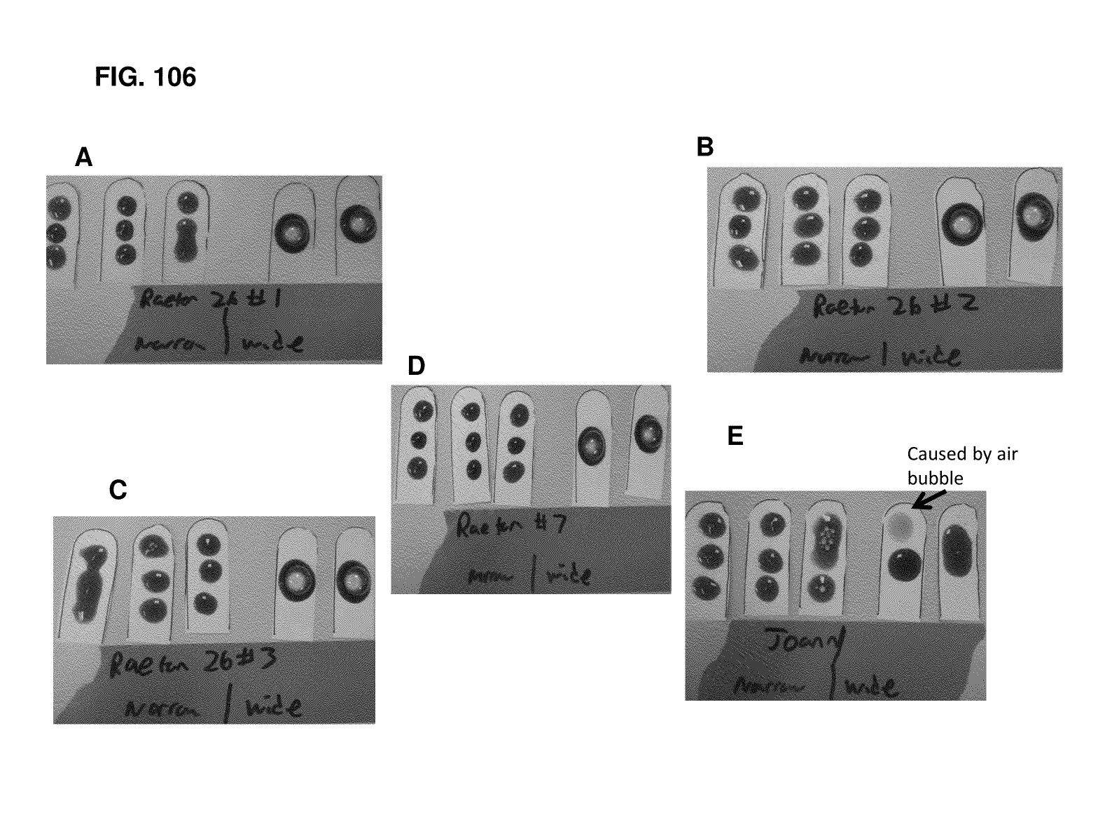

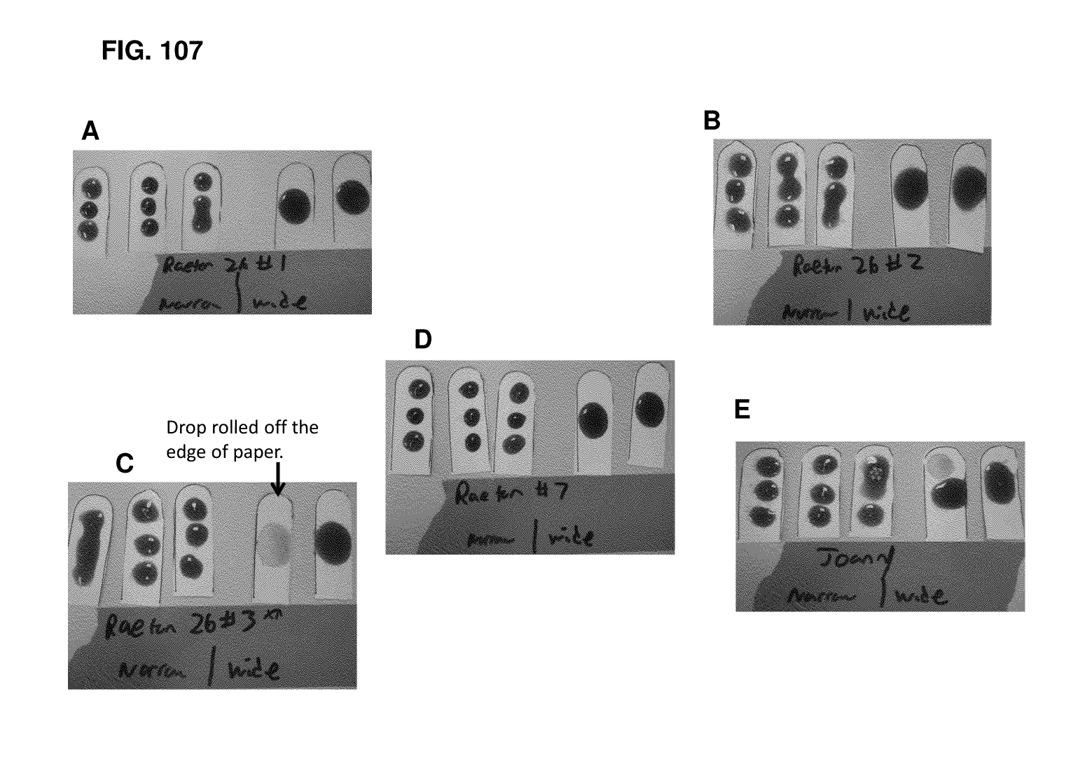

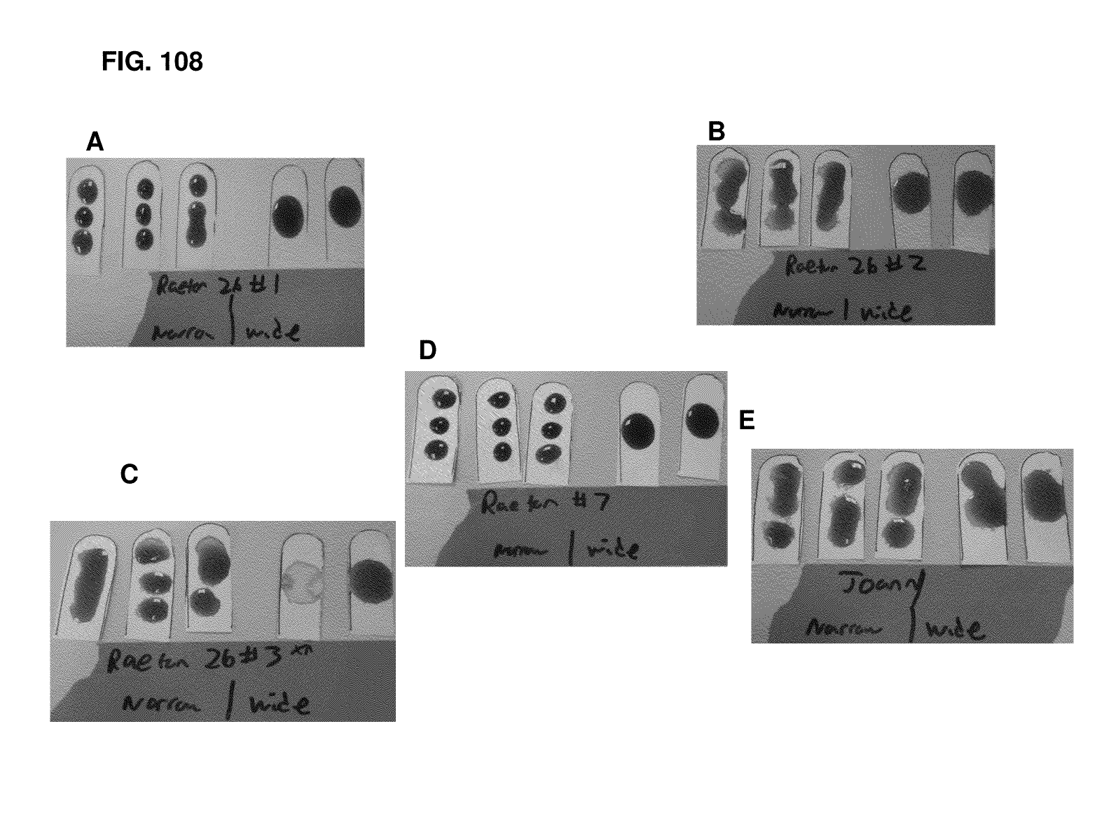

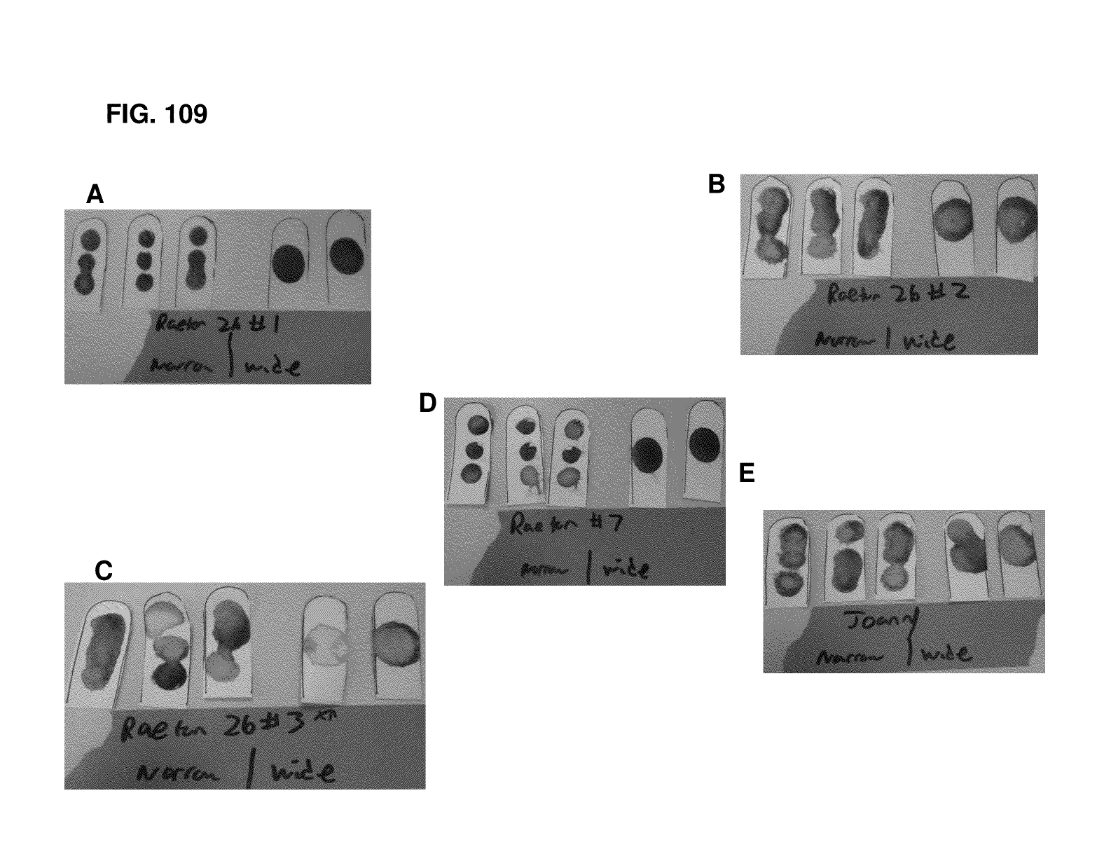

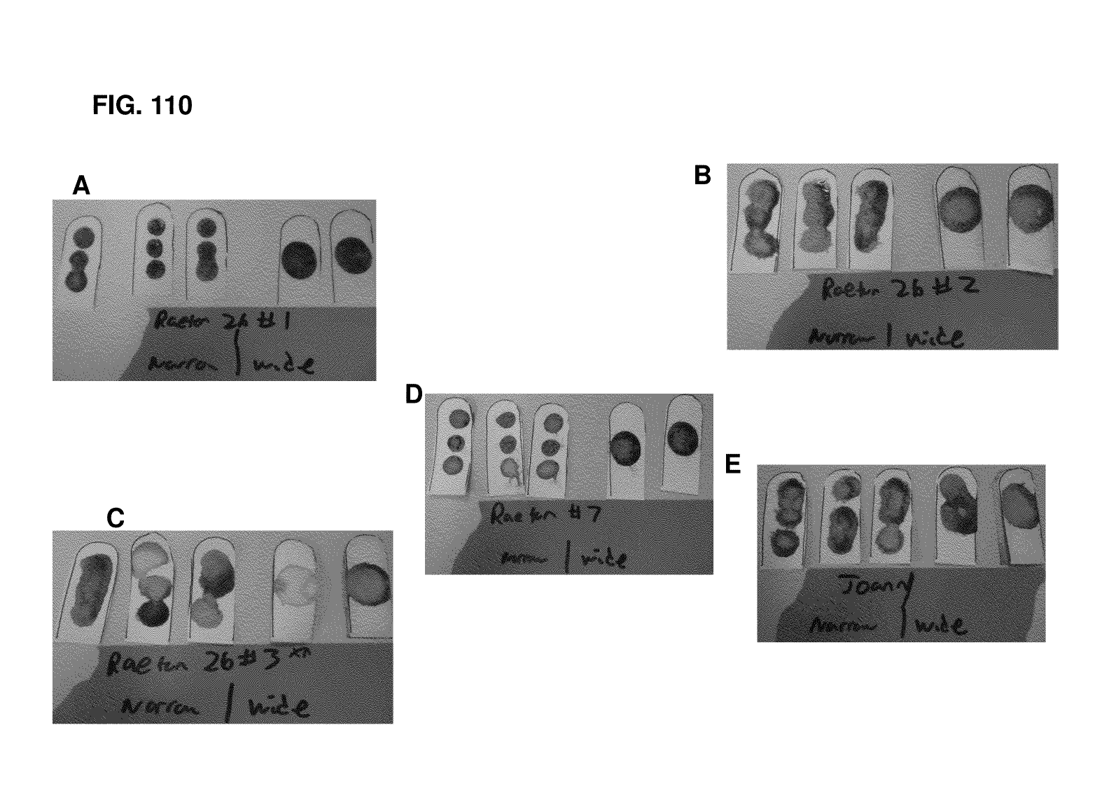

This experiment was designed to evaluate the method of application of PVP 40K PROTECT solution onto various paper liners. In this experiment, 100 μl of the solution was pipetted (3 pipette tips 33.3 μl each) onto each material as described in the Examples above. Additionally, 100 μl was pipetted into a single drop using one pipette tip which had been cut to give a wider pipetting end, and thereby a wider drop size (FIG. 105A). The paper samples include three different batches of RAETON™ 26, RAETON™ 7, and a paper sample referred to herein as Joanne (FIG. 105B). As observed before, when three drops of 33.3 μl each are used, the drops stay relatively contained. When a single drop of 100 μl each are used, the drops stay relatively contained and form a larger dome shaped drop but will remain in the same position if not disturbed (FIGS. 106-110).

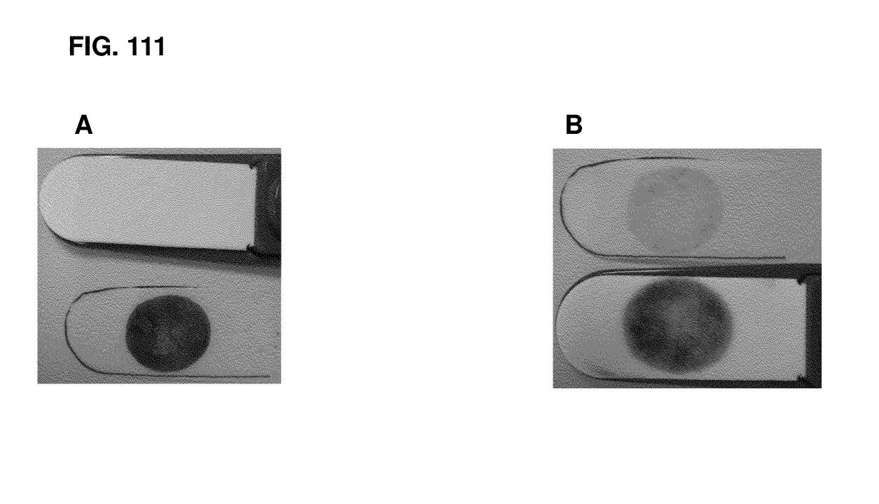

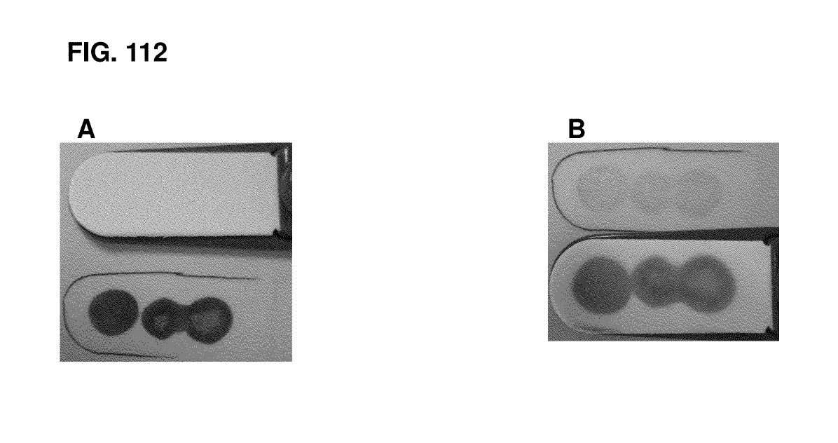

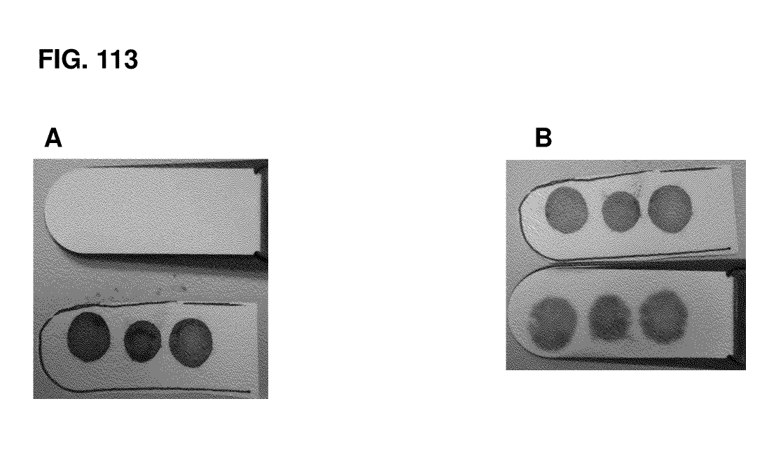

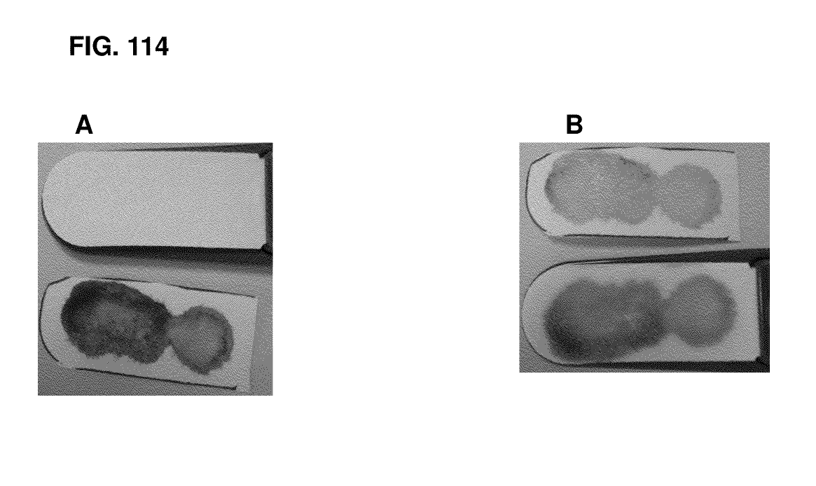

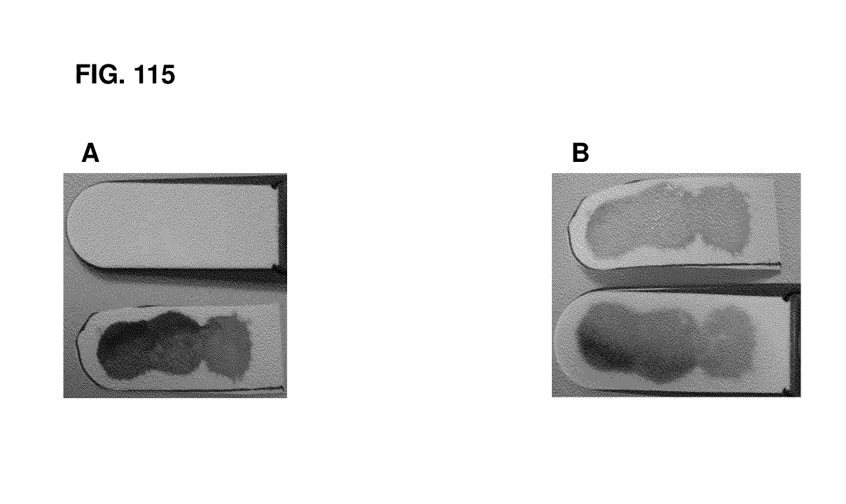

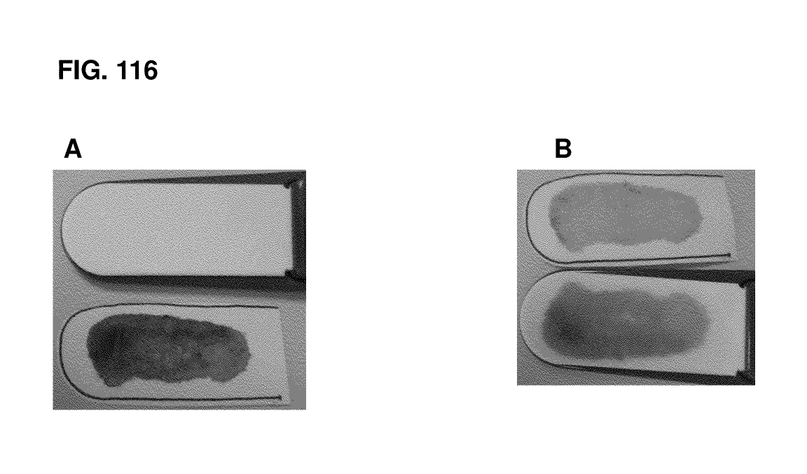

To test the transfer of solution from the various paper liners having 100 μl of solution in three versus one single drop, sample collectors were moistened with water by pipetting 50 μL water (3 pipette tips 16.66 μl each) to the paper of sample collectors. The various paper liners were then placed on top of the paper and held with moderate pressure for 5 minutes (FIGS. 111-116). Both stain sizes easily transferred to the wet collection paper. As observed before, transfer only occurred in the areas where the collector was wet.

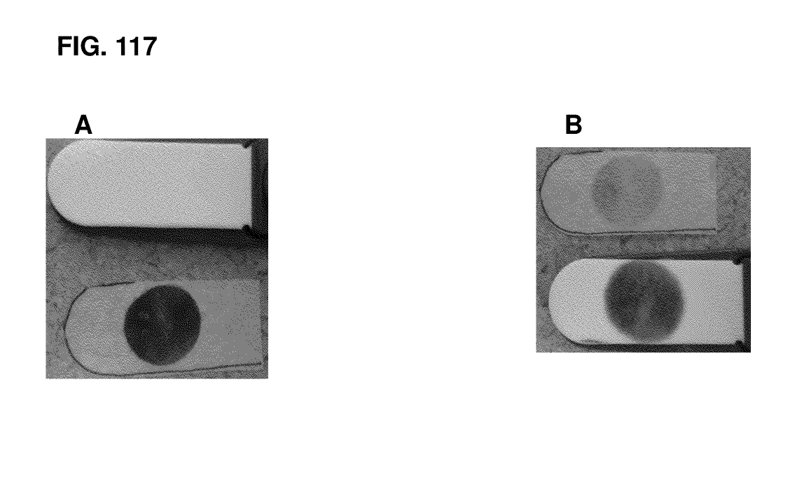

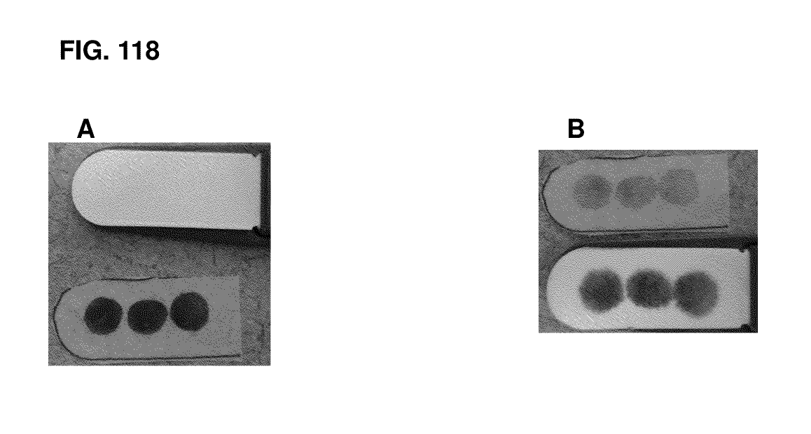

The test was repeated using Buccal DNA Collectors™ that had been used for collecting samples from a subject. Samples were collected as described above, wherein the tongue was rubbed against the cheek before sample collection. RAETON™ 26 batch #1 was used, and 100 μl of PVP 40K PROTECT solution was applied in three drops (FIG. 117) and a single drop (FIG. 118). Similar to the control studies with water, transfer and percolation were efficient, but were dependent on wetness of the collected sample.

### Example 14

### Evaluation of Excessive Pressure During Transfer of PROTECT Solution to Sample Collectors

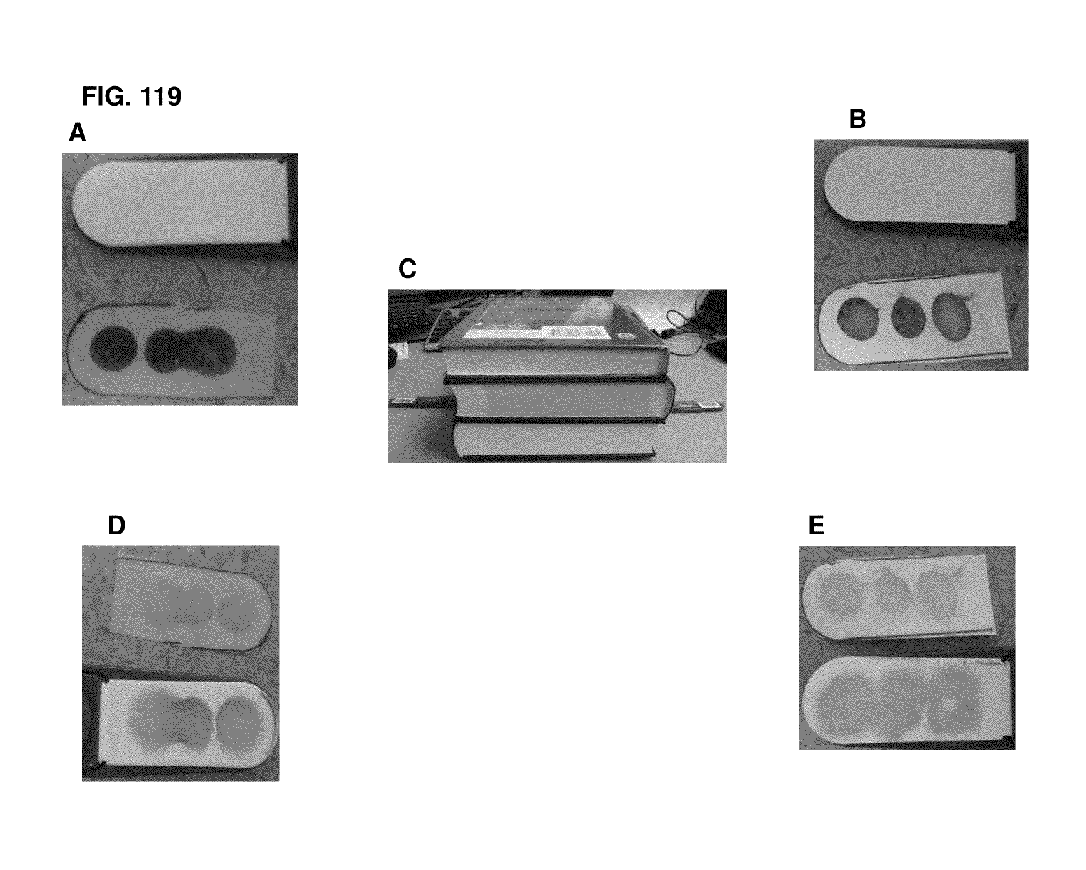

This experiment was designed to evaluate the application of excessive pressure during transfer of PVP 40K PROTECT solution to Buccal DNA sample Collectors™ that had been used for collecting samples from a subject. Samples were collected as described above, wherein the tongue was rubbed against the cheek before sample collection. In this experiment, 100 μl of the solution was pipetted (3 pipette tips 33.3 μl each) onto each material as described in the Examples above. The paper liners include RAETON™ 26 batch #1 (FIG. 119A) and RAETON™ 7 (FIG. 119B). Excessive pressure was applied overnight using textbooks stacked on top of collectors with liners applied thereon (FIG. 119C). Both paper materials transferred PROTECT solution efficiently (FIGS. 119D, E). However, RAETON™ 7 was somewhat stuck to the BDC paper causing slight ripping when removed, whereas RAETON™ 26 batch #1 was also somewhat stuck but was removed easily without ripping.

The invention illustratively disclosed herein suitably may be practiced in the absence of any element, which is not specifically disclosed herein. It is apparent to those skilled in the art, however, that many changes, variations, modifications, other uses, and applications to the method are possible, and also changes, variations, modifications, other uses, and applications which do not depart from the spirit and scope of the disclosure are deemed to be covered by the disclosure, which is limited only by the claims which follow.

What is claimed is:

1. A specimen collection device comprising:
a. a specimen collection absorbent;
b. a reagent lined holder, wherein the reagent is a stabilizing composition deposited in the reagent lined holder and wherein the stabilizing composition is able to transfer to the specimen collection absorbent;
c. a means for aligning the specimen collection absorbent with the reagent lined holder;
d. a means for contacting the stabilizing composition with the specimen collection absorbent to deliver the composition to the specimen collection absorbent; and
e. a means for sampling the specimen collection absorbent for further analysis.

2. The specimen collection device of claim 1, wherein the device further comprises a means for maintaining the chain of custody of a collected specimen.

3. The specimen collection device of claim 1, wherein the stabilizing reagent comprises at least one chelating agent, at least one surface acting agent, at least one antimicrobial agent, and combinations thereof.

4. The specimen collection device of claim 1, wherein the stabilizing composition comprises at least one chelating agent and at least one surface acting agent.

5. The specimen collection device of claim 4, wherein the stabilizing composition comprises EDTA and Tween®-20.

6. The specimen collection device of claim 5, wherein the stabilizing composition comprises 50 mM EDTA and 0.01% Tween®-20.

7. The specimen collection device of claim 1, wherein the stabilizing composition comprises at least one chelator, at least one surface acting agent, and at least one antimicrobial agent.

8. The specimen collection device of claim 7, wherein the stabilizing composition comprises EDTA, an azide stabilizer, and Tween®-20.

9. The specimen collection device of claim 8, wherein the stabilizing composition comprises 50 mM EDTA, 0.1% Sodium Azide, and 0.01% Tween®-20.

10. The specimen collection device of claim 7, wherein the stabilizing composition comprises two chelators.

11. The specimen collection device of claim 10, wherein the two chelators are EDTA and EGTA.

12. The specimen collection device of claim 7, wherein the stabilizing composition comprises two detergents.

13. The specimen collection device of claim 12, wherein the two detergents are Tween and SDS.

14. The specimen collection device of claim 7, wherein the preservative composition comprises one azide stabilizer.

15. The specimen collection device of claim 14, wherein the azide stabilizer is sodium azide.

16. The specimen collection device of claim 7, wherein the preservative composition further comprises a salt and a buffering agent.

17. The specimen collection device of claim 7, wherein the preservative composition comprises EDTA, EGTA, Tween, SDS, sodium azide, KCl, and Tris-HCl.

18. The specimen collection device of claim 7, wherein the preservative composition comprises about 5 to about 15 mM EDTA, about 1 to about 5 mM EGTA, about 0.001% to about 0.1% Tween, about 1 to about 10% SDS, about 0.01 to about 0.1 sodium azide, about 20 to about 30 mM KCl, and about 40 to about 60 mM Tris-HCl.

19. The specimen collection device of claim 1, wherein the stabilizing composition is in the form of a stabilizing dissolvable film.

20. The specimen collection device of claim 19, wherein the stabilizing dissolvable film comprises at least one film-forming polymer in addition to the stabilizing composition.

21. The specimen collection device of claim 20, wherein the at least one film-forming polymer is polyvinylpyrrolidone.

22. The specimen collection device of claim 21, wherein the polyvinylpyrrolidone is polyvinylpyrrolidone 40K.

23. The specimen collection device of claim 21, wherein the polyvinylpyrrolidone is present in an amount of about 1% to about 10% by weight in a film-forming solution used to prepare the dissolvable film.

24. The specimen collection device of claim 20, wherein the at least one water-soluble film-forming polymer is carboxy methyl cellulose.

25. The specimen collection device of claim 24, wherein the carboxy methyl cellulose is present in an amount of about 0.1% to about 1% by weight in a film-forming solution used to prepare the dissolvable film.

26. The specimen collection device of claim 19, wherein the thickness of the film is about 5μ to about 5 mm.

27. The specimen collection device of claim 19, wherein the preservative composition is present in the film formulation in an amount sufficient to deliver an amount of stabilizing composition sufficient to stabilize a collected sample.

28. The specimen collection device of claim 19, wherein the film formulation dissolves in about 5 minutes upon contacting a wet sample.

29. The specimen collection device of claim 19, wherein the dissolvable film comprises a liner.

30. The specimen collection device of claim 29, wherein the liner is selected from the group consisting of S&S® 903™ paper, S&S® IsoCode® paper, and S&S® 900™, Whatman FTA paper, RAETON™ 16 paper, RAETON™ 26 paper, RAETON™ 96 paper, RAETON™ 7 paper, RG paper, LL72 paper, and B-85 paper.

31. A specimen collection device comprising:
a. a specimen collection absorbent;
b. a reagent lined holder, wherein the reagent is a stabilizing composition deposited in the reagent lined holder and wherein the stabilizing composition is able to transfer to the specimen collection absorbent;
c. a means for aligning the specimen collection absorbent with the reagent lined holder;
d. a means for contacting the stabilizing composition with the specimen collection absorbent to deliver the composition to the specimen collection absorbent; and
e. a means for sampling the specimen collection absorbent for further analysis;
wherein the stabilizing composition comprises 50 mM EDTA and 0.01% Tween®-20.

32. The specimen collection device of claim 31, wherein the stabilizing composition is in the form of a stabilizing dissolvable film.

33. A specimen collection device comprising:
a. a specimen collection absorbent;
b. a reagent lined holder, wherein the reagent is a stabilizing composition deposited in the reagent lined holder and wherein the stabilizing composition is able to transfer to the specimen collection absorbent;
c. a means for aligning the specimen collection absorbent with the reagent lined holder;
d. a means for contacting the stabilizing composition with the specimen collection absorbent to deliver the composition to the specimen collection absorbent; and
e. a means for sampling the specimen collection absorbent for further analysis;
wherein the stabilizing composition comprises 50 mM EDTA, 0.1% Sodium Azide, and 0.01% Tween®-20.

34. The specimen collection device of claim 33, wherein the stabilizing composition is in the form of a stabilizing dissolvable film.

35. A specimen collection device comprising:
a. a specimen collection absorbent;
b. a reagent lined holder, wherein the reagent is a stabilizing composition deposited in the reagent lined holder and wherein the stabilizing composition is able to transfer to the specimen collection absorbent;
c. a means for aligning the specimen collection absorbent with the reagent lined holder;
d. a means for contacting the stabilizing composition with the specimen collection absorbent to deliver the composition to the specimen collection absorbent; and
e. a means for sampling the specimen collection absorbent for further analysis;
wherein the stabilizing composition comprises about 5 to about 15 mM EDTA, about 1 to about 5 mM EGTA, about 0.001% to about 0.1% Tween, about 1 to about 10% SDS, about 0.01 to about 0.1 sodium azide, about 20 to about 30 mM KCl, and about 40 to about 60 mM Tris-HCl.

36. The specimen collection device of claim 35, wherein the stabilizing composition is in the form of a stabilizing dissolvable film.

37. A method of collecting a specimen for analysis comprising the steps of:
a. providing a specimen collector having a specimen collection absorbent and a reagent lined cassette;
b. contacting the specimen collection absorbent to a specimen for collection;
c. closing the specimen collector, wherein the sample collection absorbent is moved into a position that aligns the specimen collection absorbent with the reagent lined cassette;
d. contacting the specimen collection absorbent to the reagent lined cassette to deliver the reagent to the specimen collection absorbent;
e. storing the specimen collector; and
f. sampling the specimen collection absorbent for analysis.
